SEQUENCE LISTING (140 sequences)

SEQ ID NO: 1 - DNA, 1613 bp, Human immunodeficiency virus type 1
agaaagagcagaagacagtggcaatgagagtgaaggggatcaggaggaattgtcagcgctggtggagatggggcatcatgctccttggaatgttgatgatctgtaatgctgtaggacaattgtgggttacggtctattatggggtacctgtgtggaaagaagccaccaccactctattccgtgcatcagatgctaaagcatatgatacagaggtacataatgtctgggccacacatgcctgtgtacccacagaccccaacccacaagaaatagaattggaaaatgtgacagaaacttttaacatgtggaaaaataacatggtagaacaaatgcatgaggatataatcagtttatgggatcaaggcctaaaaccatgtgtaaaattaaccccactccgtgttactttaaattgcactgactacaagaatgctaatagtaccaataataatagtaccagtgatagtagcaatctagaagaggagaaaggagaaataaaaaactgctctttcaatatcactacaagcataaaagataggatgcagaaagaatatgcacttttttataaacttgatatagtaccaatagataataataatactagatataggatgataagttgtaacacctcagtcattacacaggcctgtccaaaggtatcttttgagccaattcccatacattattgtgccccggctggttttgcgattctaaaatgtaaggataagaagttcaatggaacaggaccatgtagaaatgtcagcacagtacaatgtacacatggaattaggccagtagtatcaactcaattgctgttaaatggcagtctagcagaagaagaggtagtacttagatctgaaaatttcacgaacaatgctaaaaccataatagtacagctaaaagaacctataaaaatcaattgtacaagacccaacaacaatacaagaaaaagtatacatataggaccagggagagcattttatacaacaggggagataataggagacataagacaagcacattgtagcattagtaaggtagaatggaacaacactttgatacaaatagttgaaaaattaagagaacaatttgggactaaaacaataaattttactaaaccctcaggaggggacctagaaattgtaacgcacagctttaattgtagaggggaatttttctactgtaataccacaaaactgtttaatagtacttggcctgggaatattacttggactcggaataataatgttactacagaaaatatcacactcccatgcagaataaaacaaattgtgaacagatggcaggaagtaggaaaagcaatgtatgcccctcccatccaaggacaaattagatgttcatcaaatattacagggctgctattaacaagagatggtggtggggaccagaatagcacaggggagatcttcagacctggaggaggagatatgagggacaattggagaagtgaactatacaaatataaagtagtacaaattgaaccattaggaatagcacccaccaaggcaaggagaagagtggtgcagagagaaaaaagagcagtgggaacattaggagctatgttccttgggttcttgggagcagcaggaagcact

SEQ ID NO: 2 - protein, 484 aa, Human immunodeficiency virus type 1
NAVGQLWVTVYYGVPVWKEATTTLFRASDAKAYDTEVHNVWATHACVPTDPNPQEIELENVTETFNMWKNNMVEQMHEDIISLWDQGLKPCVKLTPLRVTLNCTDYKNANSTNNNSTSDSSNLEEEKGEIKNCSFNITTSIKDRMQKEYALFYKLDIVPIDNNNTRYRMISCNTSVITQACPKVSFEPIPIHYCAPAGFAILKCKDKKFNGTGPCRNVSTVQCTHGIRPVVSTQLLLNGSLAEEEVVLRSENFTNNAKTIIVQLKEPIKINCTRPNNNTRKSIHIGPGRAFYTTGEIIGDIRQAHCSISKVEWNNTLIQIVEKLREQFGTKTINFTKPSGGDLEIVTHSFNCRGEFFYCNTTKLFNSTWPGNITWTRNNNVTTENITLPCRIKQIVNRWQEVGKAMYAPPIQGQIRCSSNITGLLLTRDGGGDQNSTGEIFRPGGGDMRDNWRSELYKYKVVQIEPLGIAPTKARRRVVQREKR

SEQ ID NO: 3 - DNA, 1604 bp, Human immunodeficiency virus type 1
agaaagagcagaagacagtggcaatgaaagtgacggggatcaggaagaattgtcagcgcttgtggagatggggcatgatgctcctggggatgttaatgatctgtagtgctgcagaacaattgtgggtcacagtctattatggggtacctgtgtggaaagaagcaaccaccactctattttgtgcatcagatgctaaagcatatgacgcagagaaacataatgtttgggccacacatgcctgcgtacccacagaccccaacccacaagaaatagtattggaaaatgtgacagaatattttaatgcttggaaaaataacatggtagaacagatgcatgaggatataatcagtttatgggatcaaagcctaaaaccatgtgtaaaattaaccccactctgtgttactttaaatcgcactgatttgaataatagtactaacaccactaatagtaatagcagcggggggatgatgagagaagaaatgaaaaactgctctttcaatatcaccacaacaataggtgataggaggcaaaaagaatatgcacttttttataaacttgatatagcatcaataaaggatgatgctaataatttcacatataggttgataagttgtaacacctcagtcattacacaagcctgtccaaagatatcctttgagccaattcccatacattattgtgccccggctggttttgcaattctaaagtataacgataagaagttcaatggagaagagcaatgtaaaaatgtcagcacagtacaatgtacacatggaattaagccagtagtatcaactcagctgctgttaaatggtagtctagcagaagaagagatagtaattagatctgacaatttcacagacaatgctaaaaccataacagtacagctgaatgaatctgtagtaattaattgtacaagaccccacaacaatacaagaaaaagtataaatataggaccagggagagcatggtatacaacaggagaaataataggagatataagacaagcacattgtaacattagtaaaacacaatggaataacactttaataaagatagttaaaaaattaagagaacaatttaatacaaacaccataatctttaatcaatccacaggaggggacctagaaattgtaatgcacagttttaattgtggaggggaatttttctactgtgatacaacacaactgtttaatagtacttggaatattactggagaaagtacttggaatagtactggaaaaacaaatgaaactatcacactcccatgtagaataaaacaagttataaacatgtggcagcaagtagggaaagcaatgtatgcccctcccatcaaagggcaaattagatgttcatcaaatattacagggctgctattaacaagagatggtggtaagaacagcagtaacgggactgagacctttagacctggaggaggagatatgagggacaattggagaagtgaattatataaatataaagtagtagaaattgaaccattaggaatagcacccactaaggcaaagagaagagtggtgcagagagaaagaagagcagtaataggagctatgttccttgggttcttgggagcagcaggaagcact

SEQ ID NO: 4 - protein, 483 aa, Human immunodeficiency virus type 1
SAAEQLWVTVYYGVPVWKEATTTLFCASDAKAYDAEKHNVWATHACVPTDPNPQEIVLENVTEYFNAWKNNMVEQMHEDIISLWDQSLKPCVKLTPLCVTLNRTDLNNSTNTTNSNSSGGMMREEMKNCSFNITTTIGDRRQKEYALFYKLDIASIKDDANNFTYRLISCNTSVITQACPKISFEPIPIHYCAPAGFAILKYNDKKFNGEEQCKNVSTVQCTHGIKPVVSTQLLLNGSLAEEEIVIRSDNFTDNAKTITVQLNESVVINCTRPHNNTRKSINIGPGRAWYTTGEIIGDIRQAHCNISKTQWNNTLIKIVKKLREQFNTNTIIFNQSTGGDLEIVMHSFNCGGEFFYCDTTQLFNSTWNITGESTWNSTGKTNETITLPCRIKQVINMWQQVGKAMYAPPIKGQIRCSSNITGLLLTRDGGKNSSNGTETFRPGGGDMRDNWRSELYKYKVVEIEPLGIAPTKAKRRVVQRERR

SEQ ID NO: 5 - DNA, 1613 bp, Human immunodeficiency virus type 1
agaaagagcagaagacagtggcaatgagagtgatggggatgaggaagaattatcagcactggtggagagggggcatcttgctccttgggatgttaatgatcagtagtgctatagaaaattcgtgggtcacagtctattatggggtacctgtgtggaaagaagctaccaccactttattttgtgcatcagatgctaaagcttatgaaacagaggcacataatgtttgggccacacatgcctgtgtacccacagaccccaacccacaagaaataaaattggaaaatgtgtcagaaaattttaacatgtggaaaaataacatggtagaccaaatgcatgaggatataatcagtttatgggatcaaagcctaaagccatgtgtaaaattaaccccactctgtgttactttaaattgcactgattattttgggaatactactaataccaatactaataccaccagtagtcccagcaccaacagtagtaatgaaggagaagtgaaaaaatgctctttcaatatcaccacagaagtaagggacaaggtgcgaaaagaatttgcacttttttataaacttgatatagtacgaacaggtcatgataatactagctataggttgataagttgtaacacctcagtcattacacaggcctgcccaaagatatcctttgatccaattcccatacattattgtgccccggctggttttgcgattctcaagtgtagagataataaatttaatggaacaggaccatgtaaaaatgtcagcacagtacaatgtactcatggaattaggccagtaatatcaactcaactactgttaaatggcagtctagcagaagaagaggtagtagttagatctaaaaatttcacaaacaatgctgaagtcataatagtgcagctgaaagaatctgtacaaataaatcgtacaagacccaacaacaatacaaggaaaagtatacctatgggtccagggagagcatggtatgctacagaagatatcataggaaatataagacaggcacattgtaacattagtggagtaaaatggaataacactttacagcaaatagttaaaaaattaagagagcaatttaaaaataaaacaataaagtttcagccatcctcaggaggggacccagaaattgtaaggcacagttttaattgtagaggggagtttttctactgtgatacaacactactgtttaatagtacttggaatagtaatgatacttggaatagtactgaagggtcaaatgacactattacactcccgtgtagaataaaacaaattgtaaacatgtggcaagaagtaggaaaagcaatgtatgctcctcccatcaaaggacaacttaactgttcatcaaatattacagggccgatattaacaagagatggtggtaagggtgagaactcgaccgagaacaatactgagatattcagacctggaggaggagatatgagggacaattggcgaagtgaattatataaatataaagtagtacaaattgaaccattaggaatagcacccactaaggcaaagagaagagtggtgcagagagaaaaaagaggagcgggactgtttttcctggggttcttgggaacagcaggaagcact

SEQ ID NO: 6 - protein, 487 aa, Human immunodeficiency virus type 1
SAIENSWVTVYYGVPVWKEATTTLFCASDAKAYETEAHNVWATHACVPTDPNPQEIKLENVSENFNMWKNNMVDQMHEDIISLWDQSLKPCVKLTPLCVTLNCTDYFGNTTNTNTNTTSSPSTNSSNEGEVKKCSFNITTEVRDKVRKEFALFYKLDIVRTGHDNTSYRLISCNTSVITQACPKISFDPIPIHYCAPAGFAILKCRDNKFNGTGPCKNVSTVQCTHGIRPVISTQLLLNGSLAEEEVVVRSKNFTNNAEVIIVQLKESVQINRTRPNNNTRKSIPMGPGRAWYATEDIIGNIRQAHCNISGVKWNNTLQQIVKKLREQFKNKTIKFQPSSGGDPEIVRHSFNCRGEFFYCDTTLLFNSTWNSNDTWNSTEGSNDTITLPCRIKQIVNMWQEVGKAMYAPPIKGQLNCSSNITGPILTRDGGKGENSTENNTEIFRPGGGDMRDNWRSELYKYKVVQIEPLGIAPTKAKRRVVQREKR

SEQ ID NO: 7 - DNA, 1595 bp, Human immunodeficiency virus type 1
agaaagagcagaagacagtggcaatgagagtgaaggggatcaggaagaattatcagcacttgtggagatggggcatcatgctccttgggatgttgatgatctgtaatgctgcagaacagttgtgggtcacagtctattatggggtacctgtgtggagggatgcaaataccactctattttgtgcatccgatgctaaagcatatgatacagaggtacataatgtttgggccacacatgcctgtgtacccacagaccccaacccacaagaagtagtattggaaaatgtgacagaaagctttaacatatggaaaaataacatggtagaacaaatgcatgaggatataatcagtttgtgggatcaaagcctaaagccatgtgtaaaattaaccccactctgcgttactttaaattgcagtaatttaagtaccactaataataataccattagtggtaatgagacagcagtaaataaaggagaaataaaaaaccgctctttcaatgtcaccacaaacataagagatagggtaaagaaagaatatgcgcttttttataatcttgatttagtacaaataggtgattctaatactagctatacaatggtaaagtgtaacacctcagtcattacacaggcctgtccaaaggtaccctttgagccaattcccatacatttttgtgccccagctggttttgcgattctaaagtgtaataataagacgttcagtggaaaaggagaatgtacaaatgtcagcacagtacaatgtacgcatggaattagaccagtagtatcaactcatctgctgttaaatggcagcttagcagaagaagacatagtaattagatctgacaatttcacggacaatactaaaaccataatagtacagctagacaatactataaacattacttgtaccagacccaacaataatacaaggaaaggtatacatataggaccagggagagcattttatgcaacaggggatataataggaaatataagacaagcacattgtaaccttagtaaaacacattggaataacactttaaaacagatagttaaaaaattaagagaacaatttaaaaataaaacaatagtctttaatcaatctacaggaggggacccagaaattgtacagcacacttttaattgtagaggggaatttttctattgtaactcaacaccactgtttaatagtacttggtatcctaatagtacattggatgaaacaaacagcacagacaacaatgaaactatcacactccaatgcagaataagacaaattataaacatgtggcaggaagtaggaaaagcaatgtatgcccctcctatcagaggacaaattacatgcacatcaaatattacagggctgatattaacaagagatggtggagataacaatgaaactgagatcttcaggcctggaggaggcaatatgaaggataattggagaagtgaattatataaatataaagtagtaaaaattgagccattaggaatagcacccactaaggcaaagagaagagcggtgcagagagaaaaaagagcagcgggaataggagctgtgttccttgggttcttgggagcagcaggaagcact

SEQ ID NO: 8 - protein, 479 aa, Human immunodeficiency virus type 1
NAAEQLWVTVYYGVPVWRDANTTLFCASDAKAYDTEVHNVWATHACVPTDPNPQEVVLENVTESFNIWKNNMVEQMHEDIISLWDQSLKPCVKLTPLCVTLNCSNLSTTNNNTISGNETAVNKGEIKNRSFNVTTNIRDRVKKEYALFYNLDLVQIGDSNTSYTMVKCNTSVITQACPKVPFEPIPIHFCAPAGFAILKCNNKTFSGKGECTNVSTVQCTHGIRPVVSTHLLLNGSLAEEDIVIRSDNFTDNTKTIIVQLDNTINITCTRPNNNTRKGIHIGPGRAFYATGDIIGNIRQAHCNLSKTHWNNTLKQIVKKLREQFKNKTIVFNQSTGGDPEIVQHTFNCRGEFFYCNSTPLFNSTWYPNSTLDETNSTDNNETITLQCRIRQIINMWQEVGKAMYAPPIRGQITCTSNITGLILTRDGGDNNETEIFRPGGGNMKDNWRSELYKYKVVKIEPLGIAPTKAKRRAVQREKR

SEQ ID NO: 9 - DNA, 1676 bp, Human immunodeficiency virus type 1
agaaagagcagaagacagtggcaatgaaagtgaaggagaccaggaagaattatcaaagcttgtggagagggggcaccttgttccttggaatgttgatgatctgtagtgttacaggacaattgtgggttacagtctattatggggtacctgtgtggaaagaggcaaccaccactctattttgtgcatcaaatgctaaagcatatgatacagaggtacataatgtttgggccacacatgcctgtgtacccacagaccccaacgcacaagaagtagtattagaaaatgtgacagaatattttgacatgtggaaaaatgacatggtagaacaaatgcatgaggatgtaatcagtttatgggatcaaagcctaaagccatgtgtagaattaaccccactctgtgttactttaaattgcactgatgtgaatattactaataccaataatagtaccattaacaatagtagtaataataccaatagtagtgattgggaacggatggagccaggagaaataaaaaactgctctttcaatagcaccacaaacatgagagataggacgcagagagaatacgcacttttttataaacttgatatagaaccagtagataataaaagtaataataaaagtcttaatgaaagtattagtaaaagtattacttataggttaataagttataacacctcagtcattaaacaggcctgtccaaaagtatcttttgagccaattcccatacattattgtgccccagctggttttgcaattctaaagtgtaataatgagacattcgatggaaaaggagaatgtagaaatgtcagcacagtacaatgtacacatggaattaggccaatagtgtcaactcaactgctgttaaatggcagtctagcagaaaaggacatagtaattagatcaaacaatttctcggacaatgctaaaaccataatagtacatctgaatgaatctataacaattaagtgtataagacccaacaataatacaagaaaaagtatacatatagcaccaggaagcgcattttatgcaacaggagacataataggagatataaggcaagcacattgtaacattagtgcaaaaaattggattaacactttaaaacagatagttataaaactaaaaggaaaatataatactagtacaaaaatagactttaagccatcctcaggaggggacccagaaattgtaatgcacagctttaattgtggaggggagtttttctactgtaatacatcaaaactgtttaataatacttggaaggagaataatactttagagtcaaatgatactatggagatcattaacgaaactattatactcccatgtagaataaaacagtttataaacatgtggcagaaagtgggaaaagcaatgtatgcccctcccatcagaggacaaattaaatgtgaatcaaatattacagggctgctattaacaagagatggtggtaatacaaatagcacgaacgggaccgagaccttcagacctggaggaggaaatatgaaagacaattggagaagtgaattgtacaaatataaagtagtaaaaattgaaccaataggaatagcacccaccagggcaaaaagaagagtggtgcagagagaaaaaagagcagtgggaataggagctgtgttccttgggttcttgggagcagcaggaagcact

SEQ ID NO: 10 - protein, 506 aa, Human immunodeficiency virus type 1
SVTGQLWVTVYYGVPVWKEATTTLFCASNAKAYDTEVHNVWATHACVPTDPNAQEVVLENVTEYFDMWKNDMVEQMHEDVISLWDQSLKPCVELTPLCVTLNCTDVNITNTNNSTINNSSNNTNSSDWERMEPGEIKNCSFNSTTNMRDRTQREYALFYKLDIEPVDNKSNNKSLNESISKSITYRLISYNTSVIKQACPKVSFEPIPIHYCAPAGFAILKCNNETFDGKGECRNVSTVQCTHGIRPIVSTQLLLNGSLAEKDIVIRSNNFSDNAKTIIVHLNESITIKCIRPNNNTRKSIHIAPGSAFYATGDIIGDIRQAHCNISAKNWINTLKQIVIKLKGKYNTSTKIDFKPSSGGDPEIVMHSFNCGGEFFYCNTSKLFNNTWKENNTLESNDTMEIINETIILPCRIKQFINMWQKVGKAMYAPPIRGQIKCESNITGLLLTRDGGNTNSTNGTETFRPGGGNMKDNWRSELYKYKVVKIEPIGIAPTRAKRRVVQREKR

SEQ ID NO: 11 - DNA, 1646 bp, Human immunodeficiency virus type 1
agaaagagcagaagacagtggcaatgagagtgaaggggatcaggaagagttgcttgtggaaatggggcaccttgttccttggaatgttgatgatctgtagtgctgtagaacaattgtgggtcacagtttattatggagtacctgtgtggaaagaagcaaccaccactctattttgtgcatcagatgctaaggcatatattccagaggtacataatgtatgggccacacatgcctgtgtacccacagatcccaacccacaagaagtagaattgaaaaatgtgacagaggattttaacatgtggaagaataacatggtagaacaaatgcatgaagatgtaatcagtttatgggatcaaagcctaaagccatatgtggaattaaccccactctgtgttacgttaaattgcactgattattggggggatactactcgtgccggaaatactactgctagtgtcactagtactgctaatgtcactagtagtaaagaggtacaaatgaaaaactgctctttctatgtctccacaaacatgatggataagaaacagaaagaatacgcacttttttataaacttgatgtagtgccaataggtaatgagactaatggtaaggagactaataatagctataggttaataagttgtaacacctcagtagttacccaagcctgtccaaaggtaacctttgagccaattcccatacattattgtgccccggctggttttgtgattctaaagtgcaaggataagaggttcaatggaacaggaccatgtacaaatgtcagcacagtacaatgtacacatggaattaggccagtagtatcaactcaactactgttaaatggcagcttagcagaagaagatatagtacttagatctgaaaatttctcgaacaatgctaaaaacataatagtacagctgaatgaatctgtagtaattaattgtacaagactcaacaacaatacaagaaaaagcatacatatggggccagggaaagcattttatgcaacaggagacaccataggagatataagacaagcacattgtaacattagtgaagaggcctggaataaaactctaagacgaatagctataaaattaaaagaacaatttaatataacagacaaagtaatctttaaaccctcctcaggaggggacatagaaattgcaatgcacagtgttaattgtggaggggaatttttctactgtaatacaacacagctgtttaatagtacttggaatgaaacacagctgaatagtagtactgtgaataatattacaaggtcagacaacaacatcacactcccatgcaaaataaagcaaattgtaaacatgtggcagaaaataggaaaagcaatgtatgcccctcccatcagtggactaattagatgtaaatcaaatattacagggataatattagcaagagatggtggtaataatggcacaaatgatacgaggaccttcagacctgtaggaggaaatatgaaggacaattggagaagtgaattatataaatataaagtagtaagaattgaaccattaggagtagcacccaccaaggcaaagagaagagtggtgcagagagaaaaaagagcagtgggactaggagctatgttccttgggttcttgggagcagcaggaagcact

SEQ ID NO: 12 - protein, 498 aa, Human immunodeficiency virus type 1
SAVEQLWVTVYYGVPVWKEATTTLFCASDAKAYIPEVHNVWATHACVPTDPNPQEVELKNVTEDFNMWKNNMVEQMHEDVISLWDQSLKPYVELTPLCVTLNCTDYWGDTTRAGNTTASVTSTANVTSSKEVQMKNCSFYVSTNMMDKKQKEYALFYKLDVVPIGNETNGKETNNSYRLISCNTSVVTQACPKVTFEPIPIHYCAPAGFVILKCKDKRFNGTGPCTNVSTVQCTHGIRPVVSTQLLLNGSLAEEDIVLRSENFSNNAKNIIVQLNESVVINCTRLNNNTRKSIHMGPGKAFYATGDTIGDIRQAHCNISEEAWNKTLRRIAIKLKEQFNITDKVIFKPSSGGDIEIAMHSVNCGGEFFYCNTTQLFNSTWNETQLNSSTVNNITRSDNNITLPCKIKQIVNMWQKIGKAMYAPPISGLIRCKSNITGIILARDGGNNGTNDTRTFRPVGGNMKDNWRSELYKYKVVRIEPLGVAPTKAKRRVVQREKR

SEQ ID NO: 13 - DNA, 1586 bp, Human immunodeficiency virus type 1
agaaagagcagaagacagtggcaatgagagtgaaggggatcaggaagaattgtcagcgcttgtggaaatggggcaccatgctccttgggatgttaatgatctgtagggctgcagagcaattgtgggtcacagtctattatggagtacctgtgtggagagaagcaaacaccactctattttgtgcatcagatgctaaagcatatgatacagaggtacataatgtttgggccacacatgcctgtgtacccacagaccctaacccacaagaagtagtattggaaaatgtgacagaaaattttaacatgtggaaaaataacatggtagaacagatgcatgaggatataatcagtctatgggatcaaagcctaaaaccatgtgtaaaattaaccccactctgtgttactttaaattgtaataccattaatgccactaaagatatgataggagaattaaaaaactgctctttcaacatcaccacaagcataagagataagtggcaaaaagaatatgcacttttttataaacttgatgtagtgccaatagatgataatggtaatgatactggtaatggtagctataggctaataagttgtaatacctcagtcattacacaggcctgtccaaagacatcctttgagccaattcccatacattattgtgccccggctggttttgcgattctaaagtgtaacaataaaaagttcaatggaacaggaccacgtaaaaatgtcagcacagtacaatgtacacatggaattaggccagtagtatctactcaactgttgttaaatggcagtctagcagaagaagagatagtacttagatctgaaaatttctcaaacaatgctaaaaccataatagtacaattgaatgaatctatagtaattaattgtacaagacccaacaacaatacgagaaaaagtatacatataggaccagggagagcattttatgcagcaggagaaataataggagatataagaacagcacattgtaacattagtggaacaaaatggaataacactttaaaacagatagttgtaaaattaagagaacaatttggaaataaaacaatggtctttagtcactcctcaggaggggacccggaaattgtaaggcacagttttaattgtggaggggaatttttccattgcaatacaacacaactgtttaatagtagttggccttggaatggtactgaagggtcaaataacactgaaggaaatgacacaatcaccctcccatgcagaataaaacaaattataaacatgtggcaggaagtaggaaaagcaatgtatgcccctcccatcagaggggtaattaaatgttcatcaaatattacagggctattattaacaagagatgggggtactaacaggaccgacaatgggagcgaggtcttcagacctgggggaggagatatgagggacaattggagtagtgaattatataaaaataaagtagtaagaattgaaccattaggagtagcacccaccaaggcaaagagaagagtggtgcagagagaaaaaagagcagtgggactaggagctatgttccttgggttcttgggagcagcaggaagcact

SEQ ID NO: 14 - protein, 476 aa, Human immunodeficiency virus type 1
RAAEQLWVTVYYGVPVWREANTTLFCASDAKAYDTEVHNVWATHACVPTDPNPQEVVLENVTENFNMWKNNMVEQMHEDIISLWDQSLKPCVKLTPLCVTLNCNTINATKDMIGELKNCSFNITTSIRDKWQKEYALFYKLDVVPIDDNGNDTGNGSYRLISCNTSVITQACPKTSFEPIPIHYCAPAGFAILKCNNKKFNGTGPRKNVSTVQCTHGIRPVVSTQLLLNGSLAEEEIVLRSENFSNNAKTIIVQLNESIVINCTRPNNNTRKSIHIGPGRAFYAAGEIIGDIRTAHCNISGTKWNNTLKQIVVKLREQFGNKTMVFSHSSGGDPEIVRHSFNCGGEFFHCNTTQLFNSSWPWNGTEGSNNTEGNDTITLPCRIKQIINMWQEVGKAMYAPPIRGVIKCSSNITGLLLTRDGGTNRTDNGSEVFRPGGGDMRDNWSSELYKNKVVRIEPLGVAPTKAKRRVVQREKR

SEQ ID NO: 15 - DNA, 1618 bp, Human immunodeficiency virus type 1
agaaagagcgaagacagtggcaatgagagtgagggggatcatgaggaattatcagtacttatggaaatggggcaccatgctcctggggatattgatgatctgtaatgctagtgaaaaattgtgggtcacagtctattatggggtacctgtgtggaaagaggcaaacaccactctattttgtgcatcagatgccaaagcttatgatacagaagtacataatgtttgggccacacatgcctgtgtacccacagacccccgccctcaagaagtactattgggaaatgtgacagaaaattttaacatgtggaaaaataacatggtagaacaaatgcatgaggatataatcagtttatgggatcaaagcctaaagccatgtgtaaaattaaccccactctgtgttactttaaattgcactaacttgaatgatactaatatcagtagtagtaatgttagtacccataatagtagtggcataggagaaatgaaaaattgctctttcaatgttaccacaagtataagagataagatgaagaaagaatatgcacttttttatagacttgatatagttccaatagataatagtaacaccagttatatgttaataagttgtaatacctcagtcattacacaggcctgtccaaaggtatcctttgaaccaattcccatacattattgtgccccggctggttttgcgattctaaagtgtaatgataagaagttcaatggaacaggaccatgtaagaatgtcagcacagtacaatgtacacatggaattaggccagtagtatcaactcaactgctgttaaatggcagtttagcagaagaagagatagtaattagatctgaaaatttcacagacaatactaaaaccataatagtgcatctgaacgaatctatacaaattaattgtacaagacccaacaacaatacaagaaaaagcatacatataggaccaggaagagcattttatgcaacaggagaaataataggagatataagacaagcacattgtaaccttagtagagcaaaatggaataacacgttaaaacagatagttaaaaaattaagagtacaatttgaaaataaaacaatagtctttaatcaatcttcaggaggggacccagaaattgtaatgcacagctttaattgtggaggggaatttttctactgtaatacaacagcactgtttaatagtacttggaatagtaatagtactgaatggtcaaatgacactgaaagcaatgacacagtgattacactcccatgcagaataaaacaaatagtaaacatgtggcaggaagttggaaaagcaatgtatgcccctcccatcaagggacaaattaagtggatatcaaatattacagggatactattaacaagagatgggggaagagatgaggttaatagcacgaacgagaacaagaccgagatcttcagacctgcaggaggaaatatgaaggacaattggagaagtgaattatataaatataaagtagtaaaaattgaaccattaggaatagcacccactagggcaagaagaagagtggtgcagagagaaaaaagagcagtgacactaggagctttgttccttgggttcttgggagcagcaggaagcact

SEQ ID NO: 16 - protein, 487 aa, Human immunodeficiency virus type 1
NASEKLWVTVYYGVPVWKEANTTLFCASDAKAYDTEVHNVWATHACVPTDPRPQEVLLGNVTENFNMWKNNMVEQMHEDIISLWDQSLKPCVKLTPLCVTLNCTNLNDTNISSSNVSTHNSSGIGEMKNCSFNVTTSIRDKMKKEYALFYRLDIVPIDNSNTSYMLISCNTSVITQACPKVSFEPIPIHYCAPAGFAILKCNDKKFNGTGPCKNVSTVQCTHGIRPVVSTQLLLNGSLAEEEIVIRSENFTDNTKTIIVHLNESIQINCTRPNNNTRKSIHIGPGRAFYATGEIIGDIRQAHCNLSRAKWNNTLKQIVKKLRVQFENKTIVFNQSSGGDPEIVMHSFNCGGEFFYCNTTALFNSTWNSNSTEWSNDTESNDTVITLPCRIKQIVNMWQEVGKAMYAPPIKGQIKWISNITGILLTRDGGRDEVNSTNENKTEIFRPAGGNMKDNWRSELYKYKVVKIEPLGIAPTRARRRVVQREKR

SEQ ID NO: 17 - DNA, 1625 bp, Human immunodeficiency virus type 1
agaaagagcagaagacagtggcaatgagagtgaaggagatcaagaagaattgtcagcgcttgtggagatggggcatcatgctccttgggatattgatgatctgtagtgctacagaaaaattgtgggtcacagtctattatggggtacctgtgtggaaggaagcaaacaccactctattttgtgcatctgatgctaaagcatatgatacagaggtacataatgtttgggcaacacatgcctgtgtacccacagaccccaacccacaagaagtaagattaaaaaatgtgacagaaaattttaacatgtggaggaataacatggtagaacagatgcaggaggatataatcagtttgtgggatcaaagcctaaagccatgtgtaacattaactccactatgtgttactttaaattgcactgattattggggcaatgttactgggaccaatactactagtaaccctactggtactggtgtgggtggtaccactaacaatggcgcggaagtgatgaagtgctcttttaatgtcaccacaagtgtaagagataaggtacaaaaagaatctgctcttttttatagacttgatgtagtaaaaatagatgagaaaacaaatacaaccaattataggttgataagttgtaacacctcagtcattaaacaggcccgtccaaaggtaaactttgagccaattcccatacattattgtgccccggctggttttgcgattctaaagtgtaatgataagaagttcaatggaacaggatcatgtaaaaatgtcagcacagtacaatgtacacatggaattaagccagtagtatccactcacttgctgttaaatggcagtctagcagaagatgagatagtaattagatctgaaaatttcacgaacaatgctaaaaccataatagtacagctgaataattatgtaaaaattaattgtataagacccaataataatacaagaaaaagtatatcactcggaccaggaagagcattttatacaacaggagacataataggaaatataagacaagcacattgcaaccttagtggtacagaatggaataacactttaaaacaggtagctaacaaattaagagaacaatttaacaaaacaataataaaatttaagcaaccccccccgggaggggacctagaaatcacaatgctcacttttaattgtggaggagaatttttttactgtaattcatcagcactgtttaatagtactttgacttgggatagtaaggcatgggcaaatacacttgaagaaaatatcacactcccatgcagaataaaacaaattgtaaacaagtggcaggaagtaggaaaagcaatatatgcccctcccatcagtggacagattaattgtacatcaaatattacagggatactattaacaagagatggtggtaataacaacgacactaacaacactgaggtcttcagacctggaggaggagatatgagggacaattggagaagtgagttatataaatataaagtagtaaaaattgaaccattaggaatagcacccaccagggcaaagagaagagtggtgcagagagaaaaaagagcagcaataggagctatgttccttgggttcttgggagcagcaggaagcact

SEQ ID NO: 18 - protein, 490 aa, Human immunodeficiency virus type 1
SATEKLWVTVYYGVPVWKEANTTLFCASDAKAYDTEVHNVWATHACVPTDPNPQEVRLKNVTENFNMWRNNMVEQMQEDIISLWDQSLKPCVTLTPLCVTLNCTDYWGNVTGTNTTSNPTGTGVGGTTNNGAEVMKCSFNVTTSVRDKVQKESALFYRLDVVKIDEKTNTTNYRLISCNTSVIKQARPKVNFEPIPIHYCAPAGFAILKCNDKKFNGTGSCKNVSTVQCTHGIKPVVSTHLLLNGSLAEDEIVIRSENFTNNAKTIIVQLNNYVKINCIRPNNNTRKSISLGPGRAFYTTGDIIGNIRQAHCNLSGTEWNNTLKQVANKLREQFNKTIIKFKQPPPGGDLEITMLTFNCGGEFFYCNSSALFNSTLTWDSKAWANTLEENITLPCRIKQIVNKWQEVGKAIYAPPISGQINCTSNITGILLTRDGGNNNDTNNTEVFRPGGGDMRDNWRSELYKYKVVKIEPLGIAPTRAKRRVVQREKR

SEQ ID NO: 19 - DNA, 1643 bp, Human immunodeficiency virus type 1
agaaagagcagaagacagtggcaatgagagtgatggagatcaggaagagttatcagaacttatggagagggggcaccttgctccttgggatgttaatgatgatctgtagtgctgcagaagaatcgtgggtcacagtatattatggggtacctgtgtggaaagaagcaaccaccactctattttgtgcatcagatgctaaaggctatgatacagaaagacataatgtttgggccacacatgcctgtgtacccacagaccccaacccacaagaaattgaattggtaaatgtgacagaatattttaacatgggaaaaaataacatggtagaacagatgcatgaggatataatcagtttatgggatgaaagcctaaagccatgtgtaaaattaaccccactctgtgttactctaaattgcactaatttgaatattactaataccactggtattactaatagtagcctggaagaaatgaggagaataatgacaaactgttctttcaaggtcaccacaaatataagagataaggtgcagaagcaatatgcactgttgtataaacttgatgtagtacaaatagatgatgagagtaccacaggtaataggagtaacagcgcctacaggttgataagttgtaacacctcagtcattacacaggcccgtccaaaggtatcctttgagccaattcccatacacttttgtgccccggctggttttgcgattctaaaatgtaaggataagaagttcaatggaacaggactatgtaaaaatgtcagcacagtacaatgtacacatggaattaggccagtagtatcaactcagctgctgttaaatggcagtctagcagaagaagaggtagtaattagatctgtaaatttcacaaacaatgctaaaactataatagtacagctgaacaaatctatagaaattaattgtacaagacccaacaacaatacaagaagaggtataaatataggacccgggagagcattttacacaataaaggacataacaggagatataagacaagcacattgtaacattagtgcatcagactggaataatactgtaacacaggtagttgcaaaattaaaagagcaatttgggaataaaacaatagtctttaatcaatcctcaggaggagacccagaaattataatgcacacttttaattgtggaggggaatttttctactgtaagacaacacaactgtttaatagtacttggcctaataatggtacttggcctaatagtaattggactgataataatagaacttggaacggtgctaaaggaactatcacactcccatgcagaataaaacaaattgtaaacatgtggcaggaagtaggaaaagcaatgtatgcccctcccatcgaagggaaaataaaatgtacatcaaatcttacaggattgctattaacaagagatggtggtaatgtgaatggcaccaccatcgagaccttcagacctggaggaggagatatgagggacaattggagaagtgaattatataaatataaagtagtacaaattgaaccattaggactggcacctaccaaggcaaagagaagagtggtgcagagagaaaaaaggggagtaataggagctatgttccttgggttcttgggagcagcaggaagcact

SEQ ID NO: 20 - protein, 495 aa, Human immunodeficiency virus type 1
SAAEESWVTVYYGVPVWKEATTTLFCASDAKGYDTERHNVWATHACVPTDPNPQEIELVNVTEYFNMGKNNMVEQMHEDIISLWDESLKPCVKLTPLCVTLNCTNLNITNTTGITNSSLEEMRRIMTNCSFKVTTNIRDKVQKQYALLYKLDVVQIDDESTTGNRSNSAYRLISCNTSVITQARPKVSFEPIPIHFCAPAGFAILKCKDKKFNGTGLCKNVSTVQCTHGIRPVVSTQLLLNGSLAEEEVVIRSVNFTNNAKTIIVQLNKSIEINCTRPNNNTRRGINIGPGRAFYTIKDITGDIRQAHCNISASDWNNTVTQVVAKLKEQFGNKTIVFNQSSGGDPEIIMHTFNCGGEFFYCKTTQLFNSTWPNNGTWPNSNWTDNNRTWNGAKGTITLPCRIKQIVNMWQEVGKAMYAPPIEGKIKCTSNLTGLLLTRDGGNVNGTTIETFRPGGGDMRDNWRSELYKYKVVQIEPLGLAPTKAKRRVVQREKR

SEQ ID NO: 21 - DNA, 1626 bp, Human immunodeficiency virus type 1
agaaagagcagaagacagtggcaatgagagtgatggagatcaggaggaattatcagcgctcgtggagatggggcaccatgctccttgggatgttgatgatttatagtgctgcaggagagttatgggtcacagtttattatggggtaccggtgtggaaagaagcaaccactactttattctgtgcatcagatgctaaagcatatgacacagaggtacataatgtttgggcaacacatgcctgtgtacccacagaccctaatccacaagaagtattattggaaaatgtgacagaaaattttaacatgtggaaaaataacatggtagaacagatgcatgaggatataatcagtctatgggatcaaagcctaaagccacgtgtaaaattaaccccactctgtgttactttaaactgtactaatttgagaaatgttactaatttgaaaaatgttactaataacagtaatattagtggtactaataacaatactagtagtggggggctgaagggaggagaaatgaaaaattgctctttctatatcaccacacacagaaaggataaggtgaagaaagaatatgcacttttttataaccttgatatagtatcaacagatgatgataatacaagctatatattgagaagttgtaacacctcagtcattacccaggcctgtccaaaggtaacctttgaaccaattcccatacattattgtaccccagctggttttgcgattctgaagtgtaacgataagaagttcaatggaacaggaccatgtagaaatgtcagtacagtacaatgtacacatggaatcaagccagtagtgtcaacccaactgttgttaaatggcagtctagcagaagaagaggtagtaattagatctgaaaatttcacggacaatgttaaaaccataatagtacagctgaatgaatctgtaataattaattgtacaagacccagcaacaatacaagaaaaagtatacgttttggaccaggggcggcattttatacaacaggagacataataggagatataagacaagcacattgtaacatcagtagagcagaatggaataacactttaaaacaaatagttaaaaaattacaagaacaatttgtgaataaaacaatagtctttaatcaatctgcaggaggggacccagaaattgtaaggcacagtgtaaattgtggaggggaatttttctactgcgatacaacacaactgtttaatagtacttggaatagtactggagagtcaaataacactcaagaaaatgacctaatcacactcccatgcagaataaaacaaattataaacagatggcaggaaataggaaaagcaatgtatgcccctcccatccaaggacaaattagctgtacatcaaatattacagggctgctactaacaagagatggtggtaataataataacagcacagagaccttcagacctggaggaggaaatatgaaggacaattggagaagtgaattatataaatataaagtagtaaaaattcagccattaggggtagcacccaccaaggcaaagagaagagtggtgcagagggaaaagagcagtgggagcactaggagctatgttccttgggttcttgggagcagcaggaagcact

SEQ ID NO: 22 - protein, 489 aa, Human immunodeficiency virus type 1
SAAGELWVTVYYGVPVWKEATTTLFCASDAKAYDTEVHNVWATHACVPTDPNPQEVLLENVTENFNMWKNNMVEQMHEDIISLWDQSLKPRVKLTPLCVTLNCTNLRNVTNLKNVTNNSNISGTNNNTSSGGLKGGEMKNCSFYITTHRKDKVKKEYALFYNLDIVSTDDDNTSYILRSCNTSVITQACPKVTFEPIPIHYCTPAGFAILKCNDKKFNGTGPCRNVSTVQCTHGIKPVVSTQLLLNGSLAEEEVVIRSENFTDNVKTIIVQLNESVIINCTRPSNNTRKSIRFGPGAAFYTTGDIIGDIRQAHCNISRAEWNNTLKQIVKKLQEQFVNKTIVFNQSAGGDPEIVRHSVNCGGEFFYCDTTQLFNSTWNSTGESNNTQENDLITLPCRIKQIINRWQEIGKAMYAPPIQGQISCTSNITGLLLTRDGGNNNNSTETFRPGGGNMKDNWRSELYKYKVVKIQPLGVAPTKAKRRVVQREKS

SEQ ID NO: 23 - DNA, 1562 bp, Human immunodeficiency virus type 1
agaaagagcagaagacagtggcaatgagagtgacggggatgaggaacaattatccgcacttatggaaagaggtcaccttgctccttggaatattgatgatatgtagtgctacagaaaatttgtgggtcacagtctattatggggtacctgtgtggaaagaagcaaccaccactctattctgtgcatcggatgctaaggcatatgatacagaggcacataatgtttgggccacacatgcctgtgtacccacagaccccaacccacaagaaatgagattggaaaatgtgacagaaaattttaacatgtggaaaaataacatggtagaacagatgcaggatgatataatcagtttatgggatcaaagcctaaagccatgtgtaaaattaaccccactctgtgttactttaaattgcactaataccacaaatgctaatagtaccaataataataactgggacatgaaaaactgctctttcaatgtcacctcaggcataagagataaggtgcgaaaagaacatgcactcttttatgcacttgatgtagtaccaatagataatgagactaactataggttgataagttgtaacacctcagtcatcacacaggcctgtccaaaggtatcctttgagccaattcctatacattattatgccccggctggttttgcgattctaaaatgtagggataaaaagttcaatggaacaggaccatgtaaagatgtcagcacagtacaatgtacacatggaattaagccagtagtatcaactcaactactgttaaatggcagtctagcagaagaagaggtagtaatcagatctgaaaacttcacgaacaatgctaaaaccatattagtacaactgaatgaatctgtagtaattaattgtacaagacccaacaacaatacaagaaaaagtataaatataggaccagggagagcattctatgcaacaggagaaataataggagatataagacaagcacattgtaaccttagtaaggcacaatggaacaacactttaaaaaaggtagttgtaaaattaagagaacaatttccgaataaaacgatagtctttactcattcctcaggaggggacccagaaattgtaatgcacagttttaattgtggaggagaatttttctactgtaattcaacaccactgtttaatagtacttggaagttgaatggtactatggaatcaaatgacactgaaggaaatctcacactccaatgcagaataaaacaaatcatgaacaagtggcaggaagtaggaaaggcaatgtatgcccctcccatccaaggacagattagatgttcatcaaatattacagggctgttattagtaagagatggtggggtcaacagcgccaacgagaccttcagaccaggaggaggagatatgagggacaattggagaagtgaattatataaatataaagtagtaaaaattgaaccattaggaatagcacccaccaaggcaaagagaagagtggtgcagagagaaaaaagagcagtgggaataggagctttgttccttgggttcttgggagcagcaggaagcact

SEQ ID NO: 24 - protein, 468 aa, Human immunodeficiency virus type 1
SATENLWVTVYYGVPVWKEATTTLFCASDAKAYDTEAHNVWATHACVPTDPNPQEMRLENVTENFNMWKNNMVEQMQDDIISLWDQSLKPCVKLTPLCVTLNCTNTTNANSTNNNNWDMKNCSFNVTSGIRDKVRKEHALFYALDVVPIDNETNYRLISCNTSVITQACPKVSFEPIPIHYYAPAGFAILKCRDKKFNGTGPCKDVSTVQCTHGIKPVVSTQLLLNGSLAEEEVVIRSENFTNNAKTILVQLNESVVINCTRPNNNTRKSINIGPGRAFYATGEIIGDIRQAHCNLSKAQWNNTLKKVVVKLREQFPNKTIVFTHSSGGDPEIVMHSFNCGGEFFYCNSTPLFNSTWKLNGTMESNDTEGNLTLQCRIKQIMNKWQEVGKAMYAPPIQGQIRCSSNITGLLLVRDGGVNSANETFRPGGGDMRDNWRSELYKYKVVKIEPLGIAPTKAKRRVVQREKR

SEQ ID NO: 25 - DNA, 1643 bp, Human immunodeficiency virus type 1
agaaagagcagaagacagtggcaatgagagcgaaggggatcaggaggaattggcagcgcttgtgttggagatggggcacgatgctccttggaatgttaatgatctgtagtgctacagaaccattgtgggtaacagtctattatggggtacctgtgtggaaagaagcaaccaccactctatttcgtgcatcagatgctaaagcatatggtacagaggtacataatgtttgggccacgcatgcctgtgtacccacagaccccaacccacaagaagtagtattggaaaatgtaacagaaaattttaatgcgtgggaaaataacatggtggaacaaatgcatgaggatataatcagtttatgggatcaaagtctaaagccatgtgtaaagttaaccccactctgtgttactttaaaatgcactgataatttggggaatgatactaaaaccagtaataagagctgggaaaagatggagccaggagaaataaaaaactgctccttcaacatcaccacaagcataggagataagacgcaggaaacatatgcatttttttataaacttgatgtagtaccaatagataataagactacaatagataataatactgcaagaaactatagcgactataggttgataagttgtaacacctcagtcattacacaggcctgtccaaaggtatcttttgaaccaattcccatacattattgtgccccggctggttttgcgattctaaagtgtaacaataagacattcatgggaaaaggaccatgtacaaatgtcagcacagtacaatgtacacatggaattaagccagtagtatcaactcaactgctgttaaatggcagtctggcagaagaagagataataattagatctgaaaatttcacggacaatgctaaaaccttaataatacatctgaaccactctgtagaaattaagtgtataagacccaacaacaatacaagcgaaggtatacatataggaccagggagagcgttttatccaacaagaataataggagatataagaaaagcacattgtaacattaatgaaacagcatggaagacaactttagcacagatagttacaaaattaagagaacaatttgggaataaaacaatagtctttagccaatcctcaggaggggacccagaaattgtaatgcacagttttaattgtggaggggaatttttctactgtgatacaacaaaactgtttaatagtacttggaatgttaatgatacttggaatggtgctggagggtcaaacagcactgaaagaaacaccaccatcatactcccatgcaaaataaaacaaattataaacttgtggcaggaggtaggaaaagcaatgtatgcccctcccatcaaaggactaattagatgttcatcaaatattacagggctgctattaacaagagatggtggtaataacaatgacacaaacgggacagagatcttcagacctgggggaggagatatgagggacaattggagaagtgaattatataaatataaagtagtgaaaattgaaccattaggagtagcacccactaaggcaaagagaagagtggtgcagagagaaagaagagcagtgggaataggagctttgttccttgggttcttgggagcagcaggaagcact

SEQ ID NO: 26 - protein, 494 aa, Human immunodeficiency virus type 1
SATEPLWVTVYYGVPVWKEATTTLFRASDAKAYGTEVHNVWATHACVPTDPNPQEVVLENVTENFNAWENNMVEQMHEDIISLWDQSLKPCVKLTPLCVTLKCTDNLGNDTKTSNKSWEKMEPGEIKNCSFNITTSIGDKTQETYAFFYKLDVVPIDNKTTIDNNTARNYSDYRLISCNTSVITQACPKVSFEPIPIHYCAPAGFAILKCNNKTFMGKGPCTNVSTVQCTHGIKPVVSTQLLLNGSLAEEEIIIRSENFTDNAKTLIIHLNHSVEIKCIRPNNNTSEGIHIGPGRAFYPTRIIGDIRKAHCNINETAWKTTLAQIVTKLREQFGNKTIVFSQSSGGDPEIVMHSFNCGGEFFYCDTTKLFNSTWNVNDTWNGAGGSNSTERNTTIILPCKIKQIINLWQEVGKAMYAPPIKGLIRCSSNITGLLLTRDGGNNNDTNGTEIFRPGGGDMRDNWRSELYKYKVVKIEPLGVAPTKAKRRVVQRERR

SEQ ID NO: 27 - DNA, 1613 bp, Human immunodeficiency virus type 1
agaaagagcagaagacagtggcaatgagagtgaaggggatcaggaagaattgtcagctcttgtggaaatggggcaccatgctccttgggatgttgatgatctgtagtgctgcagaacaactgtgggtcacagtctattatggggtacctgtgtggaaagatgcaaccaccactttattttgtgcatcagatgctaaagcatacgacaaagaggcacataatgtttgggccacacatgcctgtgtacccacagaccctaacccacgagaaataaaattggaaaatgtgacagaaaattttaacatgtggaaaaatgacatggcagaccagatgcatgaggatataatcagtttatgggatcaaagcctaaagccatgtgtagaattaaccccactctgtgttactttaaattgcactaatattagtttgaatagtactaacaatgatactattaacagtagtaatagtactgaaggaataaatatgagggaagaaatgaaaaactgctctttcaataccaccacaagtataggagataagaataagagagaatatgcacttttttataaacttgatgtagtaccaatagataataagacaagctatacgttgataaattgtaacacctcagtcattaaacaggcctgtccaaaggtaacctttgaaccaattcccatacattattgtgccccggctggttttgcgattctaaagtgtctcaataagacgttcgatggaaatggaacatgtacaaatgtcagcacagtacagtgtacacatggcattagaccagtagtgtcaacccaactactgttaaatggcagtctagcagaagaagaggtagtaattagatatgagaatgtccaggacaatactaaaaccataatagtacagctgaacgaaactgtaaaaattaattgtacaagacccaacaacaatacaagaaaaggtatacatgtgggatgggggagaccaatttatgcaacaggagaaataataggagatataagacaagcacatcgtaatctaagtaaaaaagactggggagacactttaaagaagatagctataaaactacaagaacaatttaatacaacaataatctttgagcaatcctcaggaggggacccagaaattacaatgcacagtcttaattgtggaggggaatttttctactgtaatacatcaaagctgtttaatggcacttggtctaatggtacttggactagtggtatttggaataatactggagagtcagatagcacaatcacactcccatgcagaataaaacaaattataaacaggtggcagggagtaggacaagcaatgtataaccctcccatcaacggactaattagctgttcatcaaatattacaggactgatattaacaagagatggaggtaacaacaggtccaacgagaccttcagaccaagtggaggaaacatgagggacaattggagaagtgaattatataaatatagagtagtaaaaattgaaccattaggagtagcacccaccaaggcaaagagaagagtggtgcagagagaaaaaagagcagtggggatgataggagctgtgttccttgggttcttgggagcagcaggaagcact

SEQ ID NO: 28 - protein, 484 aa, Human immunodeficiency virus type 1
SAAEQLWVTVYYGVPVWKDATTTLFCASDAKAYDKEAHNVWATHACVPTDPNPREIKLENVTENFNMWKNDMADQMHEDIISLWDQSLKPCVELTPLCVTLNCTNISLNSTNNDTINSSNSTEGINMREEMKNCSFNTTTSIGDKNKREYALFYKLDVVPIDNKTSYTLINCNTSVIKQACPKVTFEPIPIHYCAPAGFAILKCLNKTFDGNGTCTNVSTVQCTHGIRPVVSTQLLLNGSLAEEEVVIRYENVQDNTKTIIVQLNETVKINCTRPNNNTRKGIHVGWGRPIYATGEIIGDIRQAHRNLSKKDWGDTLKKIAIKLQEQFNTTIIFEQSSGGDPEITMHSLNCGGEFFYCNTSKLFNGTWSNGTWTSGIWNNTGESDSTITLPCRIKQIINRWQGVGQAMYNPPINGLISCSSNITGLILTRDGGNNRSNETFRPSGGNMRDNWRSELYKYRVVKIEPLGVAPTKAKRRVVQREKR

SEQ ID NO: 29 - DNA, 1646 bp, Human immunodeficiency virus type 1
agaaagagcagaagacagtggcaatgagagtgaaggggatcaggaagaactatcagcacttgtggaaatggggcaccttgctccttgggatatcgatgatctgtagtgctaaagaagacaagttgtgggtcacagtctattatggggtacctgtgtggagagatgcaaacaccactctattttgtgcatcaggtgctaaagcatataagacagaggtacataatgtctgggccacacatgcctgtgtacccacagaccccaacccacaagaagtggtattgggaaatgtgacagaatattttaatgcatggaaaaatgacatggtagaacagatgcatgaggatataatcaatctatgggatcaaagcctaaagccatgtgtaaaattaaccccactctgtgtcactttaaattgcactaacgttaaaaacaatgctaccaaaataaatgataccaccactacacctagtgaggaaatagaaataaaaaactgctctttcaacatcaccgcaggcataagagataagatacagaaagaatatgcattgttttctaaatttgatttagtacaaatccatgaagataataaaaataataataatacaaactatacagactataggttgataagttgtaacacctcagtcattacgcaggcctgtccaaaagtatcctttgagccaattcccatacatttttgtaccccggctggttttgcgattctaaagcgtaataataagacattcaacggaaaagggccatgtacaaatgtcagtacagtacagtgtacacatggaattaggccagtagtatcaactcaactgctgctaaatggcagtttagcagaagaggatgtagtaattagatctgaaaatttcacaaacaatgttaaaaccataatagtacagctgaaagaagctgtacaaattaattgcacaaggcccaacaacaatacaagaaaaagtatacctataggaccagggagagcattttatgcaacaggagacataataggagatataagacaagcacattgtaacattagtggaacacaatggaataaaactttaggaaagatagttgaaaaattaaaagaacaatttgggaataaaacaataatctttaaccaacccgtaggaggggacccagaaattgtagcgcacacttttaattgtggaggggaatttttctactgtaatacaacacctctgtttaatagtacctggacttggaatagtacttggaatggtactacaagtactgggaatgttactaaaaaaattatcacactccaatgcagaataagacaaattgtaaacatgtggcagaaagtaggaaaagcaatgtatgcccctcccatcagaggacagattggatgttcatcaaatattacagggctgctattaacaagagatggtggtaatagtgagaacgggaccaataacacagacacagagaccttcagaccgggaggaggagatatgagggacaattggagaagtgaattatataaatataaagtagtaagaattgaaccattaggaatagcacccactaaggcaaggagaagagtggtgcagagagaaaaaagagcagtgggaataggggctttgttccttgggttcttgggagcagcaggaagcact

SEQ ID NO: 30 - protein, 496 aa, Human immunodeficiency virus type 1
SAKEDKLWVTVYYGVPVWRDANTTLFCASGAKAYKTEVHNVWATHACVPTDPNPQEVVLGNVTEYFNAWKNDMVEQMHEDIINLWDQSLKPCVKLTPLCVTLNCTNVKNNATKINDTTTTPSEEIEIKNCSFNITAGIRDKIQKEYALFSKFDLVQIHEDNKNNNNTNYTDYRLISCNTSVITQACPKVSFEPIPIHFCTPAGFAILKRNNKTFNGKGPCTNVSTVQCTHGIRPVVSTQLLLNGSLAEEDVVIRSENFTNNVKTIIVQLKEAVQINCTRPNNNTRKSIPIGPGRAFYATGDIIGDIRQAHCNISGTQWNKTLGKIVEKLKEQFGNKTIIFNQPVGGDPEIVAHTFNCGGEFFYCNTTPLFNSTWTWNSTWNGTTSTGNVTKKIITLQCRIRQIVNMWQKVGKAMYAPPIRGQIGCSSNITGLLLTRDGGNSENGTNNTDTETFRPGGGDMRDNWRSELYKYKVVRIEPLGIAPTKARRRVVQREKR

SEQ ID NO: 31 - DNA, 1606 bp, Human immunodeficiency virus type 1
agaaagacagaagacagtggcaatgagagcgaaggggaccaggaagaattgtcagcacttgtggtggagatggggcaccatgctctttgggatgttgatgatctgtagtgctgcaaaagaaaaattgtgggtcacagtctattatggggtgcctgtgtggaaagaagcaaccaccactctattttgtgcatcagatgctaaagcatatgacacagaagcacataatgtttgggccacacatgcttgtgtacccacaaaccctaacccacaagaagtattattgaaaaatgtgacagaagattttaacatgtggaaaaataatatggtagaacagatgcatgaggatataatcagtttatgggatcaaagcctaaagccatgtgtgaaattaaccccactctgtgttactttacattgcactgatgcgaacattactgcaaacagtactgctactaacagtactgttagctccattaaagaagaagtgaaaaactgctctttcaatatcaccacagaagtaagagacaaggtaaagaaagaacatgcacttttttatagacttgatgtagtaccaatagctaatgataatacaagctatacattggtaaattgtaacacctcaaccattacacaggcctgtccaaaggtgacctttgaaccaattcctatacattattgtgccccggctggttttgcgattctaaaatgtaatgataagaatttcaatggaacaggaccatgtaaaaatgtcagcacagtacaatgcacacatggaattaggccagtggtgtcaactcaactactgttaaatggcagtctagcagaagatgaggtagtaattagatctgaaaatttcacaaacaatgcaaaaatcataatagtacagctaaatgaatctgtaataattaattatacaagacctggcaacaatacaagaaaaagtatacatataggaccgggaagtgcattttatgcaacaggagacataataggagatataagacaagcacattgtaacattagtaaagcagattgggagaaaacactaaaacaggtagttaaaaaattacaggaacaatatgggaataaaacaataaactttacccaatcctcaggaggagacccagaaattgtaatgcacagtcttaattgtggaggagaatttttctattgtaatacaacaaagctgtttaatagtacttggcagaatggtactattgtaggatcagaaaatacgtcagacattatcatactcccatgcagaataaagcaaattataaacaggtggcaggaagtaggaaaagcaatgtatgcccctcccatcagcggagacattagatgtacatcaaatattacagggctgctattaacaagagatgggggtataaagaacaagaccaatgggacagagacagagatcttcagacctgcaggaggagatatgaaggacaattggagaagtgaattatataaatataaagtagtaaaaattgaaccgttaggaatagcacccaccagggcaaggagaagagtggtgcagagggaaaaaagagaagtgacgctgggagttatgttccttgggttcttgggagcagcaggaagcact

SEQ ID NO: 32 - protein, 482 aa, Human immunodeficiency virus type 1
SAAKEKLWVTVYYGVPVWKEATTTLFCASDAKAYDTEAHNVWATHACVPTNPNPQEVLLKNVTEDFNMWKNNMVEQMHEDIISLWDQSLKPCVKLTPLCVTLHCTDANITANSTATNSTVSSIKEEVKNCSFNITTEVRDKVKKEHALFYRLDVVPIANDNTSYTLVNCNTSTITQACPKVTFEPIPIHYCAPAGFAILKCNDKNFNGTGPCKNVSTVQCTHGIRPVVSTQLLLNGSLAEDEVVIRSENFTNNAKIIIVQLNESVIINYTRPGNNTRKSIHIGPGSAFYATGDIIGDIRQAHCNISKADWEKTLKQVVKKLQEQYGNKTINFTQSSGGDPEIVMHSLNCGGEFFYCNTTKLFNSTWQNGTIVGSENTSDIIILPCRIKQIINRWQEVGKAMYAPPISGDIRCTSNITGLLLTRDGGIKNKTNGTETEIFRPAGGDMKDNWRSELYKYKVVKIEPLGIAPTRARRRVVQREKR

SEQ ID NO: 33 - DNA, 1589 bp, Human immunodeficiency virus type 1
agaaagagcagaagacagtggcaatgaaagtgaaggggaccaggaagagttatcagcacttgtggagatggggcatcatgccccttgggatgttgacgatttgtagtgttgcagaacaattgtgggtcacagtctattatggggtacctgtgtggaaagaagccaccaccactctattttgtgcatcagaagctaaggcatatgttacagaggtacataatatttgggccacacatgcctgtgtacccacagaccccaacccacaagaagcagtattggaaaatgtgacagaaaattttaacatatggaaaaatgacatggtagaccagatgcatgaggatataatcagtgtatgggatcaaagcctaaagccatgtgtgaaattaaccccgctctgtgtcactttaaattgcactgattattttgggaaaactaatattactaccacttctagcagtggtcccaataatgatagaggaatgaaaaactgctctttcaatatcaccacaagcataagagataaggtaacgaaagaacatgcacttttttatagagttgatgtagtcccaatagatagtagtaatagtagctatagattgataaattgtaacacctcagtcattacacaggccagtccaaaagtatcctttgagccaattcccatacattattgtaccccggctggttttgcgattataaagtgtaataataagacattcaatggaacaggaccatgtagaaatgtcagcacagtacaatgtacacatggaattaggccaatagtgtcaactcagctgttgttaaatggcagtctagcagtagaagaggtagtaattagatctgaaaatatcacgaacaatgctaaaaccataatagtacaattgaacgaatctgtaagcatcaattgtacaagacccagcaacaatacaagaagggggatacatatgggaccagggagagcattttggacaacaggtgaagtaataggagatataaggaaagcacattgtaacattagtagaaaagaatggaatgacactttagacaaggtagtcaaaaaattaagggaaaaatttaatgcaacaataatctttaatcaatccacaggaggggacccagaaattgtaatgcacacttttaattgtggaggggagtttttctactgtaacacatcacaactgtttaatagtacttgggatattaatggaaatactactggagggttagaaggcaatgacacaatcacactccaatgtagaataaaacaaattgtaaacatgtggcaggaagtaggaaaagcaatgtatgcccctcccatccaaggaaaaattagatgttcatcaaatattacagggctgctattaacaagagatggtggtaataacagtagtaacaatgagaccttcagacctggaggaggagatatgagggacaattggagaagtgaactatataaatataaagtagtaaaaattcaaccactaggaatagcacccaccaaggcaaagagaagagtggtgcagagagaaaaaagagcagtgggaataggagctttgttccttgggttcttgggagcagcaggaagcact

SEQ ID NO: 34 - protein, 477 aa, Human immunodeficiency virus type 1
SVAEQLWVTVYYGVPVWKEATTTLFCASEAKAYVTEVHNIWATHACVPTDPNPQEAVLENVTENFNIWKNDMVDQMHEDIISVWDQSLKPCVKLTPLCVTLNCTDYFGKTNITTTSSSGPNNDRGMKNCSFNITTSIRDKVTKEHALFYRVDVVPIDSSNSSYRLINCNTSVITQASPKVSFEPIPIHYCTPAGFAIIKCNNKTFNGTGPCRNVSTVQCTHGIRPIVSTQLLLNGSLAVEEVVIRSENITNNAKTIIVQLNESVSINCTRPSNNTRRGIHMGPGRAFWTTGEVIGDIRKAHCNISRKEWNDTLDKVVKKLREKFNATIIFNQSTGGDPEIVMHTFNCGGEFFYCNTSQLFNSTWDINGNTTGGLEGNDTITLQCRIKQIVNMWQEVGKAMYAPPIQGKIRCSSNITGLLLTRDGGNNSSNNETFRPGGGDMRDNWRSELYKYKVVKIQPLGIAPTKAKRRVVQREKR

SEQ ID NO: 35 - DNA, 1703 bp, Human immunodeficiency virus type 1
agaaagagcagaagacagtggcaatgagagtgagggagaccaggaagaattatcagcacttgtggagatggggcaacatatggagatggggcatgatgctccttgggatgttgatgatctgtagtgctgcagaagatttgtgggtcacagtttattatggggtacctgtgtggaaagacgcaaagaccactctattttgtgcatcagatgctaaagcatataagacagaggtacataatgtttgggccacgcatgcctgtgtacccacagaccccaacccacaagaagtagaaatgaaaaatgtgacagaagattttaacatgtggaaaaataatatggtagaacagatgcatgaggatataatcagtttatgggatcagagcctaaaaccacgtgtaaaattaaccccactctgtgttactttaaagtgctttgatgtgaagaataaaaccactactaccactactaatagtaccacatccactattagtactactaccactaagacgcccactgttagtaaagggacagagaaatcagaactgacaaactgctctttcaatatcaccacaaacataagagataagtttcagaaaaactatgcaatttttgataaacttgatgtagtaccaatagatgatgataatgatactactactaacaataatactagtaatgaaaaaagctttaggttaataaattgtaacacctcagtcatcacacaggcctgcccaaagatatcatttgaaccaattcccatacattattgtaccccggctggttttgcgattctaaagtgtaaagataaaaatttcaatggaacaggaaaatgtaaaagcgtcagcacagtgcaatgtacacatggaattaggccagtagtgtcaactcaactactgttaaatggcagtctagcagaagaagaggtagtaattagatctgccgatttctcggacaatactaaaatcataatagtacagctgaataaatctgtagaaattaattgtacaagacccaacaacaataaaagaaaaagtataaatataggaccagggagagcaatgtttgcaacaggagacataataggagatataagaaaagcatcttgtaccattaatgaaacacaatggaataacacgttacaacaggtagttataaaattaaaagaacaatatggaaataaaacaatagtctttgaccgcccctcaggaggggacccagaaattgtaatgcacagttttaattgtggaggagaatttttctattgtaattcaacacaactgtttaatagtagttgggggcctaatggtactcggaatggtactacaacgataaatggtactatcatactcccatgtagaataaaacaaattataaacatgtggcaggaagtaggaaaagcaatgtatgcccctcccatcgagggacttattaactgtacatcaaatatcacagggctactattaacaagagatggtggccatgacaataatgacacaaaaaataacaataccgagatcttcagacctggaggaggagatatgagggacaattggagaagtgaattatataaatataaagtagtaaaaattgaaccattaggaatagcacccaacaggacaaaaagaatagtggtgcaaagagaaaaaagagcagtgggattcggagctgtgttccttgggttcttgggagcagcaggaagcact

SEQ ID NO: 36 - protein, 509 aa, Human immunodeficiency virus type 1
SAAEDLWVTVYYGVPVWKDAKTTLFCASDAKAYKTEVHNVWATHACVPTDPNPQEVEMKNVTEDFNMWKNNMVEQMHEDIISLWDQSLKPRVKLTPLCVTLKCFDVKNKTTTTTTNSTTSTISTTTTKTPTVSKGTEKSELTNCSFNITTNIRDKFQKNYAIFDKLDVVPIDDDNDTTTNNNTSNEKSFRLINCNTSVITQACPKISFEPIPIHYCTPAGFAILKCKDKNFNGTGKCKSVSTVQCTHGIRPVVSTQLLLNGSLAEEEVVIRSADFSDNTKIIIVQLNKSVEINCTRPNNNKRKSINIGPGRAMFATGDIIGDIRKASCTINETQWNNTLQQVVIKLKEQYGNKTIVFDRPSGGDPEIVMHSFNCGGEFFYCNSTQLFNSSWGPNGTRNGTTTINGTIILPCRIKQIINMWQEVGKAMYAPPIEGLINCTSNITGLLLTRDGGHDNNDTKNNNTEIFRPGGGDMRDNWRSELYKYKVVKIEPLGIAPNRTKRIVVQREKR

SEQ ID NO: 37 - DNA, 1658 bp, Human immunodeficiency virus type 1
agaaagagcagaagacagtggcaatgaaagtgaaggggatcaagaagagttatcagcacttgttgagatggggcgccatgctccttgggatgttaatgatctgtagtgctgcagaacaattgtgggtcacagtctattatggggtacctgtgtggagagaagcaaacaccactctattttgtgcatcagatgctaaagcatatgataaagaggtacataatgtttgggccacacatgcctgtgtacccacagaccccaacccacaagaagtagaattggaaaatgtgacagaaaattttaacatgtggaaaaatgacatggtagaacagatgcatgaggatataatcagtttatgggatcaaagcctaaagccatgtgtaaaattaaccccactctgcgttactttaaattgtactgatttaaggtcacagaatgtgacttataccactggtgctaataccactatggctactaccactagtactaataccactagtagtgggggagagatgcaggtaggaatgaaaaactgctctttcaatatcaccacaaacacacaagataaggtgaagggatatgcacattttgataaccttgatctagtacaaatagaggatgaaaatcacagcaataacagctataggttgatacattgtaacacctcagtaattacacaggcctgtccaaaggtatcctttgagccaattcctatacattatcgtgccccggctggttttgcgattctaaagtgtaaagataagaagttcaatggaacaggaccctgtacaaatgtcagcacagtacagtgtacacatggaattaggccagtagtatccactcaactgctgttcaatggcagtctagcagaagaagaggtagtaattagatctgccaatttctcagaaaatgataaaatcataatagtacagctgaaagacgctgtacaaattaattgtacaagacccaacaacaacaccagaaaaggtatacatatgggaccagggaaagtattttacgcaacagaagtcataggggacataaggcgagcacattgtaacattagtaaagaaaattggaataatactttaaaacagatagctatacaattaagagagcaagagcagttcaagaataaaacaatagtctttaatcaatcctcaggaggggacccagaaattgtaatgtctagttttaattgtggaggggaatttttctactgtaatacaacacaactgtttaatagtacttgggagaatgatactagtacttggaatgatactgaagggtcgaatggcactatcacactcccatgcagaataaaacaaattatcaacatgtggcaggaggtaggaaaagcaatatatgcccctcccatcaaaggaccacttcattgttcatcaaatattacagggctactattaacaagagatggtggtaatactaatgagagcaacaccaccgaggtcgaggtcttcagacctttaggaggaaacatgagggacaattggagaagtgaattatataaatataaagtagtaaagattgaaccattaggaatagcacccaccaaggcaaagagaagagtggtgcagagagaaaaaagagcagtgggaataggagctgtgttccttgggttcttgggagcagcaggaagcact

SEQ ID NO: 38 - protein, 500 aa, Human immunodeficiency virus type 1
SAAEQLWVTVYYGVPVWREANTTLFCASDAKAYDKEVHNVWATHACVPTDPNPQEVELENVTENFNMWKNDMVEQMHEDIISLWDQSLKPCVKLTPLCVTLNCTDLRSQNVTYTTGANTTMATTTSTNTTSSGGEMQVGMKNCSFNITTNTQDKVKGYAHFDNLDLVQIEDENHSNNSYRLIHCNTSVITQACPKVSFEPIPIHYRAPAGFAILKCKDKKFNGTGPCTNVSTVQCTHGIRPVVSTQLLFNGSLAEEEVVIRSANFSENDKIIIVQLKDAVQINCTRPNNNTRKGIHMGPGKVFYATEVIGDIRRAHCNISKENWNNTLKQIAIQLREQEQFKNKTIVFNQSSGGDPEIVMSSFNCGGEFFYCNTTQLFNSTWENDTSTWNDTEGSNGTITLPCRIKQIINMWQEVGKAIYAPPIKGPLHCSSNITGLLLTRDGGNTNESNTTEVEVFRPLGGNMRDNWRSELYKYKVVKIEPLGIAPTKAKRRVVQREKR

SEQ ID NO: 39 - DNA, 1568 bp, Human immunodeficiency virus type 1
agaaagagcagaagacagtggcaatgagagtgaaggggatcatgaagaattgtcagcacttgtggtggagatggggcatgatgctccttgggatgttgatgatctgtagtgctacagaacaattgtgggtcacagtctattatggggtacctgtgtggaaagaagcaaccaccactctattttgtgcatcagatgctaaagcatatgacacagaggcacataatgtttgggccacacatgcctgtgtacccacagaccctaacccacaagaagtagaattgaaaaatgtgacagaaaatcttaacatgtggaaaaatgacatggtagaacagatgcatgaggatataatcaatttatgggatcaaagcctaaagccatgtgtaaaattaactccactctgtgtcactttacattgcactaatttgaatgttactaccagtaatactacaagttggggagagatggaggcaggagaaataaaaaactgctctttcaatgtcaccacacgcagaagaaataagaaagaatatgcacttttttataaacttgatgtagtacctatagatagtgataatgcaagctatacgttgataaattgtaacacttcagtcattacacaagcctgtccaaaggtatcctttgaaccaattcccatacattattgtgccccggctggttttgcgattctaaaatgtaatgataagaaattcaatggaacaggaccatgtaaaaatgttagcacagtacaatgtacacatggaattaggccagtagtgtcaattcaactgctgttaaatggcagtctagcagaagaagaggtagtaattagatctgaaaatttctcgaacaatgctaaagccgtaatagtacagctgaatgcatctatagaaattaattgtacaagacccaacaacaatacaagaaaagatatacatataggaccagggagagcattatatacaacaggaggaataataggagatataagacaagcacattgtagccttaggaaagcagaatggaatgacactttaaaacatgtagttacaaaattaagagaacaatttgggaataaaacaatattctttaatcaatcctcaggaggggacccagagattgtaatgcacagttttaattgtggaggggaatttttctactgtaatacaacaatgctgtttaatagtaatagtacttggaatgatactacaggaccagataataacactatcatactcccatgtagaataaaacaaattataaacaggtggcaggaagtaggaaaagcaatgtatgcccctcctatcagtggaccaattaaacgcacatcaaatattacagggctactattaacaagagatggtggtagtaacaccaccgagaccttcagacctggaggaggagatatgagggacaattggagaagtgaattatataaatataaagtagtaaaaattgagccattaggggtagcacccaccagggcaaggagaagagtggtgcagagagaaaaaagagcagtgggactgggagctgtattccttgggttcttgggagcagcaggaagcact

SEQ ID NO: 40 - protein, 469 aa, Human immunodeficiency virus type 1
SATEQLWVTVYYGVPVWKEATTTLFCASDAKAYDTEAHNVWATHACVPTDPNPQEVELKNVTENLNMWKNDMVEQMHEDIINLWDQSLKPCVKLTPLCVTLHCTNLNVTTSNTTSWGEMEAGEIKNCSFNVTTRRRNKKEYALFYKLDVVPIDSDNASYTLINCNTSVITQACPKVSFEPIPIHYCAPAGFAILKCNDKKFNGTGPCKNVSTVQCTHGIRPVVSIQLLLNGSLAEEEVVIRSENFSNNAKAVIVQLNASIEINCTRPNNNTRKDIHIGPGRALYTTGGIIGDIRQAHCSLRKAEWNDTLKHVVTKLREQFGNKTIFFNQSSGGDPEIVMHSFNCGGEFFYCNTTMLFNSNSTWNDTTGPDNNTIILPCRIKQIINRWQEVGKAMYAPPISGPIKRTSNITGLLLTRDGGSNTTETFRPGGGDMRDNWRSELYKYKVVKIEPLGVAPTRARRRVVQREKR

SEQ ID NO: 41 - DNA, 1541 bp, Human immunodeficiency virus type 1
agaaagagcagaagacagtggcaatgagagtgaaggagatgaggaagcactggcagcactggtggacagggggcatcatgctccttgggatgctgatgatctgtagtgctgtaaacaacttgtgggtcactgtatattatggagtgcctgtgtggagagaagcaaccaccaccctattttgtgcatcagatgctaaagcatacaaaacagaggtacataatgtctgggccacacatgcctgtgtacccacagaccccaacccacaagagatagatttggtaaatgtgacagaaaattttaacatgtggaaaaataacatggtagaacagatgcatgaggatataatcagtttatgggatcaaagcctaaaaccatgcgtaaaattaaccccactctgtgttgtgaaaaactgctctttcaacaccaccacaatggtaagggatagggagcggaaagaatatgctctcttttataaacttgatgtagtacaaatgaatgataataataataatagtacccatggaacctatagattgataaattgtaatacctcagtcattacacaggcctgtccaaaggtatcctttgagccaattcccatacattattgcgccccggctggttttgcgattctaaagtgtaaagacaagaagttcaatggaacgggaccatgcaaaaatgtcagcacagtacagtgtacacatggaattaggccagtagtgtcaactcaactgctgctaaatggcagtctagcagaagaagaggtaataattagatctgaaaacttcacaagcaatgctaaaaccataatagtacagctaaatgaaactgtagagattaattgcacaagacctagcaacaatacaagaagaagtatacatataggaccagggagagcattttacacaacaggagacataataggagatataagaaaagcacattgtaacattagtagaacaaaatggaataacactttaggacagatagttgaaaaattacaagaacaatttaagaataaaacaataatctttaattcatcctcaggaggggacccagaaattgtatatcacagttttaattgtggaggggaatttttctactgtaatacaacagaactgttcgatagtacctggtatagcccctggaacagtactggtgggtcaaataacactgaagggaatagcacgatcacactcaaatgcagaataaagcaaattgtaaacaggtggcaggaagtaggaaaagcaatgtatgcccctcccatccagggaaaaattaaatgttcatcaaatattacagggctactattaacaagagacggtggtaatagtaatagtagtaacgagaccttcagaccaggaggaggagacatgagagacaattggagaagtgaattatataaatataaagtaataaaaattgaaccattaggagtagcacccaccaaggcaaagagaagagtggtgcaaagagaaagaagagcagtgggaacaataggagctatgttccttgggttcttgggagcagcaggaagcact

SEQ ID NO: 42 - protein, 460 aa, Human immunodeficiency virus type 1
SAVNNLWVTVYYGVPVWREATTTLFCASDAKAYKTEVHNVWATHACVPTDPNPQEIDLVNVTENFNMWKNNMVEQMHEDIISLWDQSLKPCVKLTPLCVVKNCSFNTTTMVRDRERKEYALFYKLDVVQMNDNNNNSTHGTYRLINCNTSVITQACPKVSFEPIPIHYCAPAGFAILKCKDKKFNGTGPCKNVSTVQCTHGIRPVVSTQLLLNGSLAEEEVIIRSENFTSNAKTIIVQLNETVEINCTRPSNNTRRSIHIGPGRAFYTTGDIIGDIRKAHCNISRTKWNNTLGQIVEKLQEQFKNKTIIFNSSSGGDPEIVYHSFNCGGEFFYCNTTELFDSTWYSPWNSTGGSNNTEGNSTITLKCRIKQIVNRWQEVGKAMYAPPIQGKIKCSSNITGLLLTRDGGNSNSSNETFRPGGGDMRDNWRSELYKYKVIKIEPLGVAPTKAKRRVVQRERR

SEQ ID NO: 43 - DNA, 1625 bp, Human immunodeficiency virus type 1
agaaagagcagaagacagtggcaatgagagtgaaggggatcaggaagagttatcaaaacttgtggaaatggggcacattgctccttgggatattgatgattagtagtgctacagaacaattgtgggtcacagtctattatggggtacctgtgtggaaagaagcaaccaccactctattttgtgcatcagatgctaaagcctataatacagaggttcataatgtttgggccacacatgcctgtgtacccacagaccccaatccacaagaagtaatgttaaatgtgacagagaattttaacatgtggaaaaatgacatggtagaacagatgcaggaggatataatcagtttatgggatcaaagcctaaagccatgtgtaaaattaaccccactctgtgttactttaagctacactgatgcaaatagtactgatgttaatcataccaaaaatagtagtgagggaatgatggagggagaaaaaatgaaaaactgctctttcaatatcaccacaagcatgggaaataagatgcagaaagaatatgcactttttcatagacttgatgtaataccaatggataatgaaagtgctagtgctaactattctaactataggttaataagctgtaacacctcagtcactacacaggcttgtccaaaaatatcatttgagccaattcccatacattattgtaccccggctggttttgcgattctaaaatgtaatgataagaaattcagtggaaaaggaggatgtagaaatgtcagtacagtacaatgcacacatggaattaagccagtagtatcgactcaactactgttaaatggcagtctagcagaagaagatgtggtaattaaatctgccaatttctcggacaatgctaaaaccataatagtacagctgaatgaatctgtaataattaattgtacaagacccaacaacaatacaagaaagggtatacatatgggaccagggaaaacattttatgcaacaggagccataataggagatataagacaagcacattgcaatgttagtagaacagaccggaataacactttaaaaagggtagctaaaaaactacaagaacaatttaatacaacaaaagttgtctttaaacaatcctcaggaggggacccagaaattgtaatgcacagttttaattgtggaggggaatttttctactgtaatacatcagggctgtttaatagtacttggccttggaatgatactaaagaggcaaataacactaacacaatcacactcccatgcaaaataaaacaaatcataaacatgtggcaggcagtagggaaagcaatgtatgcccctcccattagtgggataattaaatgtgaatctaatattacagggctgctactaacaagagatggtggtagtaagaacacaacagatagtaatgacacaaacataacacaagaggtcttcagaccaggaggaggagatatgagggacaattggagaagtgaattatataaatataaagtagtaagaattgaaccattaggagtagcacccactaaggcaaaaagaagagtggtgcagagagaaaaaagagcagtgggaataggagctgtgttccttgggttcttgggagcagcaggaagcact

SEQ ID NO: 44 - protein, 489 aa, Human immunodeficiency virus type 1
SATEQLWVTVYYGVPVWKEATTTLFCASDAKAYNTEVHNVWATHACVPTDPNPQEVMLNVTENFNMWKNDMVEQMQEDIISLWDQSLKPCVKLTPLCVTLSYTDANSTDVNHTKNSSEGMMEGEKMKNCSFNITTSMGNKMQKEYALFHRLDVIPMDNESASANYSNYRLISCNTSVTTQACPKISFEPIPIHYCTPAGFAILKCNDKKFSGKGGCRNVSTVQCTHGIKPVVSTQLLLNGSLAEEDVVIKSANFSDNAKTIIVQLNESVIINCTRPNNNTRKGIHMGPGKTFYATGAIIGDIRQAHCNVSRTDRNNTLKRVAKKLQEQFNTTKVVFKQSSGGDPEIVMHSFNCGGEFFYCNTSGLFNSTWPWNDTKEANNTNTITLPCKIKQIINMWQAVGKAMYAPPISGIIKCESNITGLLLTRDGGSKNTTDSNDTNITQEVFRPGGGDMRDNWRSELYKYKVVRIEPLGVAPTKAKRRVVQREKR

SEQ ID NO: 45 - DNA, 1606 bp, Human immunodeficiency virus type 1
agaaagagcagaagacagtggcaatgagagcgaaggagatcaagaggaattgtcagcactcgtggagatggggcatcatgctccttgggatgttaatgatctatagtactgcagaaaaaacgtgggtcacagtatattatggggtacctgtgtggaaggaagcaaacaccactctattttgtgcatcagatgctaaagcatatgatacagaggcacataatgtttgggccacacatgcctgtgtacccacagaccccaacccacaagaaataaagttggaaaatgtgacagagaactttaacatgtggaaaaacaacatggtagaacagatgcatgaggatattatcagtttatgggatcaaagcctacagccaagtgtaaaattaaccccactttgtgttactttaaattgctctactgcgaattttactaaaaggaattttactaatagcactgagcatgaaaagccgagtgcagaaatgagaaactgctctttcaatatcaccacaatcgtaagagataaggtaacaaaagaacatgcacttttttatagagatgatgtagtaccaatagataatgctagtaataataccagttataggttaataaattgtaatacctcagtcattacacaggcctgtccaaagatatcctttgagccaattcctatacattattgtgccccggctggttttgcgattctaaagtgtaatgataagacatttaatggaacagggctatgtaaaaatgtcagcacagtacaatgtacacatggaattagaccagtagtgtcaactcaactgttgttaaatggcagtctagcagaaaaaaaggtagtagttagatctgaggagttttcagacaatgctaaatccataatagtacagctgaatacatctgtagtaattaattgtacaagacccggcaataatacaagaagaagcatacctatgggaccaggaagagtattttatgcaacagatataataggagacataagacaggcacattgtaaccttagtagagcagcatggaataacactttaaagctgatagccgcagaattaaaagaaatatataataaaacaatagcctttaatcgatcctcaggaggggacccagaaattgtaatgcacacttttaattgtgggggggaatttttctactgtaatacaacacaactgtttaatagtgcttggaacagtactaatatttatactgggaataattctactaatgacaccatctctaatgacgccatctcactcccatgtagaataaaacaaattgtaaacatgtggcaggaagtaggaaaagcaatgtatgcccctcccatcagaggaaacatcagctgttcctcaaatattacagggctgatattgacaagagatggcgggaatagtagtagtaataccgagatcttcagacctcagggagggaatataaaggacaattggagaagtgaattatataaatataaagtagtaagaattgaaccattaggattagcacccaccaaggcaaagaggagagtggtgcggagagaaaaagagcggtaacgttcggagctttgttccttgggttcttgggagcagcaggaagcact

SEQ ID NO: 46 - protein, 483 aa, Human immunodeficiency virus type 1
STAEKTWVTVYYGVPVWKEANTTLFCASDAKAYDTEAHNVWATHACVPTDPNPQEIKLENVTENFNMWKNNMVEQMHEDIISLWDQSLQPSVKLTPLCVTLNCSTANFTKRNFTNSTEHEKPSAEMRNCSFNITTIVRDKVTKEHALFYRDDVVPIDNASNNTSYRLINCNTSVITQACPKISFEPIPIHYCAPAGFAILKCNDKTFNGTGLCKNVSTVQCTHGIRPVVSTQLLLNGSLAEKKVVVRSEEFSDNAKSIIVQLNTSVVINCTRPGNNTRRSIPMGPGRVFYATDIIGDIRQAHCNLSRAAWNNTLKLIAAELKEIYNKTIAFNRSSGGDPEIVMHTFNCGGEFFYCNTTQLFNSAWNSTNIYTGNNSTNDTISNDAISLPCRIKQIVNMWQEVGKAMYAPPIRGNISCSSNITGLILTRDGGNSSSNTEIFRPQGGNIKDNWRSELYKYKVVRIEPLGLAPTKAKRRVVRREKE

SEQ ID NO: 47 - DNA, 1613 bp, Human immunodeficiency virus type 1
agaaagagcagaagacagtggcaatgaaagtgaaggggatcaggaagaattatcagtgcttgtggggatggggcaccatgctcctcgggatattgatgatctgtagtgctgcagaaaatttgtgggtcacagtctactatggggtacctgtgtggaaagaagcaaccaccactctatttcgtgcatcagatgctaaagcatatgatacagaggtacataatgtttgggccacacatgcctgtgtacccacagaccccaacccacaagaagtagtattggaaaatgttacagaaaattttaatatgtggaaaaatgacatggtagaacagatgcaggaggatatagtcagtttatgggatcaaagcctaaagccatgtgtagaatcaactccactctgtgttactctaaattgtactgatgtgaagaagaatgctaataataccactggtaataccactgatggtaacgtggaaaggttggagaaagaagaaataaaaaactgctctttcaatatcaccacaagcataagagataagatgcggaaagaatatgcacttttttatagccttgatgtagtaccaatagataaggataatacaagctataggttgataagttgtaacacctcagtcattacacaggcctgtccaaaagtatcctttgagccaattcccatacattattgtgccccggctggttttgcgcttctaaaatgtaatgataaggagttcaatggaacaggaccatgtaggaatgtcagcacagtccaatgtacacatggaattaggccagtagtatcaactcaactgctgttaaatggcagtctagcagaagaaaagatagtaattagatctgagaatttcacgagcaatgctaaaactataatagtacagctgaataaatctatagaaattaattgtataagacccaacaacaacacaagaagaagtatacatataggaccagggggagcattttatgcaacagaaataataggagatataagacaagcacattgtaccctcaatagaacagaatggaataacactctaggacagatagttaaaaaattaagagaacaatatgggaataaaacaataaaatttacgcagccctcctcaggaggggacccagaagttgtaatgcacagttttaattgtggaggggaatttttctactgtaattcatcacagctgtttaatagtacttgggatgttactgaagggtcaaataacactgaaggaagcaacgatacaggaatcatactcccgtgcagaataaaacaaataataaacatgtggcagaaagtaggaaaagcaatgtatgcccctcccatcagaggacaaattaactgtacatcaaatattacagggctactattaataagagatggtggtaacaacgggaccgacaacgggaccgagaccttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcact

SEQ ID NO: 48 - protein, 485 aa, Human immunodeficiency virus type 1
SAAENLWVTVYYGVPVWKEATTTLFRASDAKAYDTEVHNVWATHACVPTDPNPQEVVLENVTENFNMWKNDMVEQMQEDIVSLWDQSLKPCVESTPLCVTLNCTDVKKNANNTTGNTTDGNVERLEKEEIKNCSFNITTSIRDKMRKEYALFYSLDVVPIDKDNTSYRLISCNTSVITQACPKVSFEPIPIHYCAPAGFALLKCNDKEFNGTGPCRNVSTVQCTHGIRPVVSTQLLLNGSLAEEKIVIRSENFTSNAKTIIVQLNKSIEINCIRPNNNTRRSIHIGPGGAFYATEIIGDIRQAHCTLNRTEWNNTLGQIVKKLREQYGNKTIKFTQPSSGGDPEVVMHSFNCGGEFFYCNSSQLFNSTWDVTEGSNNTEGSNDTGIILPCRIKQIINMWQKVGKAMYAPPIRGQINCTSNITGLLLIRDGGNNGTDNGTETFRPGGGDMRDNWRSELYKYKVVKIEPLGVAPTKAKRRVVQREKR

SEQ ID NO: 49 - DNA, 1616 bp, Human immunodeficiency virus type 1
agaaagagcagaagacagtggcaatgagagtgaaggggatcaggaggaattatcagtgcttgtggacatggggcacgatgctccttgggatgttgatgatctgtagtgctgtagaacaattgtgggtcacagtctattatggggtgcctgtgtgggaagaagcgaccaccactctattttgtgcatcagatgctaaaacatatgatccagaggtacataatgtttgggccacacatgcctgtgtacccacagaccccaacccacaagaagtagtattgggaaatgtgacagaaaactttaacatgtggaaaaatgacatggtaaaccaaatgcatgaggatataatcagtttatgggatcaaagtttaaagccatgtgcaaaattaaccccactctgtgttactctaaattgcactgataagttgaatattaatactaccagtaccaatagtagtaccaataatactactagtagtggagtggatgaagggggaatgaaaaactgctctttcaatgtcaccacaagcataagagatagggtgcagaaagaatatgcacttttttataaacctgatgtagtaccaatagatgataatactaataatactagctataggttgataaattgtaacacctcagtcattacacaagcctgtccaaaggtaacctttgatccaattcccatacattattgtgccccggctggttttgcgattctaaaatgtaacaataagacgttcaatggatcaggaccatgtacaaatgtcagcacagtacaatgtacacatggaattaaaccagtggtgtcgactcaattgctgttaaatggcagtctagcagaggaagaaatagtaatcaggtctgaagatttcacggacaatgatagaaccataatagtacagctgaatgaatctgtagtaattcattgtacaagacccaacaacaatacaagaaaaagtatacacctaggaccagggagtgcattttatgcaacaggagatataataggagatataaaacaagcacattgtaacattagtagagcaaattggactaacaccttaaaacagatagctggaaaattaaaagaacagtttggaaataagacaatattctttaatcaatcctcaggaggggatccagaaattgtaacacacagtttcaattgtagaggggaatttttctactgtaatacatcacaattgtttaacagtacttggcttcataataatagtactgggaatgatactgaaaagaatggtaatatcacactcccacgcagaataaaacaaattataaacatgtggcagcaagtaggaaaagcaatgtatgccccccctgtcaaaggactaattacatgttcatcaaatattacaggactgctattagtaagagatggtggtaacaacaccaacgccaccgacaccgagaccttcagacctggaggaggagatatgagggacaattggagaagtgaattatataaatataaagtagtaaaaatcaaaccattaggaatagcacccaccaaggcaaaaagaagagtggtgcagagagaaaaaagagcagcactaggagctatgttccttgggttcttgggagcagcaggaagcact

SEQ ID NO: 50 - protein, 487 aa, Human immunodeficiency virus type 1
SAVEQLWVTVYYGVPVWEEATTTLFCASDAKTYDPEVHNVWATHACVPTDPNPQEVVLGNVTENFNMWKNDMVNQMHEDIISLWDQSLKPCAKLTPLCVTLNCTDKLNINTTSTNSSTNNTTSSGVDEGGMKNCSFNVTTSIRDRVQKEYALFYKPDVVPIDDNTNNTSYRLINCNTSVITQACPKVTFDPIPIHYCAPAGFAILKCNNKTFNGSGPCTNVSTVQCTHGIKPVVSTQLLLNGSLAEEEIVIRSEDFTDNDRTIIVQLNESVVIHCTRPNNNTRKSIHLGPGSAFYATGDIIGDIKQAHCNISRANWTNTLKQIAGKLKEQFGNKTIFFNQSSGGDPEIVTHSFNCRGEFFYCNTSQLFNSTWLHNNSTGNDTEKNGNITLPRRIKQIINMWQQVGKAMYAPPVKGLITCSSNITGLLLVRDGGNNTNATDTETFRPGGGDMRDNWRSELYKYKVVKIKPLGIAPTKAKRRVVQREKR

SEQ ID NO: 51 - DNA, 1613 bp, Human immunodeficiency virus type 1
agaaagagcagaagacagtggcaatgagagtgagggggaccaggaagaattatcagcacttgtggagatggggcatcatgctccttgggatgtcaatgatctgtaatgctacaaaagatttgtgggtcacagtttattatggggtacctgtatggaaagaagcaaacaccagtctattttgtgcatcagatgctaaagcatatgatacagaggtacataatgtttgggccacacatgcctgtgtacccacagaccccaacccacaagaagtattcatgaacaatgtgacagaaaattttaacatgtggaaaaataacatggtagaacaaatgcatgaggatataatcagtttatgggatcaaagcctaaaaccatgtgtaaaattaaccccactcggtgttactctagattgcactaaggctaatattaccaataatagtaccactaatagtagcgggggaatagaggagggaagagacatagaaaattgctctttcaatatcaccacaaacataagagataagataaagaaagaatatgcacttttttatagccttgatgtgatagcaatagatgatagtagtaatagtagtaatagaagctataggttgagaggttgtaacacctcaaccatcacacaggcctgtccaaaggtaacctttgagccaattccaatacattattgtgccccagctggttttgcgattctaaagtgtaacgataagaagttcaatggaacaggaccatgtaaaaatgtcagtacagtacaatgtacacatggaattaggccagtagtatcaactcaactgctgctaaatggcagtatagcagaaaaagaggtagtaattaggtccgctaatttcacggacaatgctaaaaccataatagtacagctgaataactctgtacaaattaattgcacaagacccggcaacaatacaagaaaaagtatacatataggaccaggcagagcattttatgcacatgaaataataggggagataagacaagcacattgtacccttaacagaacacaatggaataacactttaaaacagatagttataaaattaagagaacaatttaacaataagacaatagtctttaatcactcctcaggaggggacccagaaattgtaacacacagttttaattgtggaggggaatttttctactgtaatacatcacaattatttaatagtacctggaggagtaatgaaactgtaaatgacactatgggaaaggacacaaatgacacaacaatcacactcccatgcagaataaaacaaattataaacatgtggcaggaagtaggaaaagcaatgtatgccccgcccatcagaggacaaattagctgttcatcaaatattacagggctgctattaacaagagatggtggtgtgaacgagaccaacgccaccgaggtcttcagacctggaggaggagatatgagggacaattggagaagtgaattatataaatataaagtagtagaaattgaaccattaggaatagcacccaccaaggcaaagagaagagtggtgcagagagagaaaagagcagtgggaataggagctgtgttccttgggttcttgggagcagcaggaagcact

SEQ ID NO: 52 - protein, 485 aa, Human immunodeficiency virus type 1
NATKDLWVTVYYGVPVWKEANTSLFCASDAKAYDTEVHNVWATHACVPTDPNPQEVFMNNVTENFNMWKNNMVEQMHEDIISLWDQSLKPCVKLTPLGVTLDCTKANITNNSTTNSSGGIEEGRDIENCSFNITTNIRDKIKKEYALFYSLDVIAIDDSSNSSNRSYRLRGCNTSTITQACPKVTFEPIPIHYCAPAGFAILKCNDKKFNGTGPCKNVSTVQCTHGIRPVVSTQLLLNGSIAEKEVVIRSANFTDNAKTIIVQLNNSVQINCTRPGNNTRKSIHIGPGRAFYAHEIIGEIRQAHCTLNRTQWNNTLKQIVIKLREQFNNKTIVFNHSSGGDPEIVTHSFNCGGEFFYCNTSQLFNSTWRSNETVNDTMGKDTNDTTITLPCRIKQIINMWQEVGKAMYAPPIRGQISCSSNITGLLLTRDGGVNETNATEVFRPGGGDMRDNWRSELYKYKVVEIEPLGIAPTKAKRRVVQREKR

SEQ ID NO: 53 - DNA, 1658 bp, Human immunodeficiency virus type 1
agaaagagcagaagacagtggcaatgagagcgaaggggatcaggaagaattggcagcacttgtggagatggggcaccatgctcccatggggcaccatgctccttgggatgttaatgatgatctgtagtgctgcagaagaaaaatgggtcacagtctattatggggtacctgtgtggaaagaagcaaccaccactctattttgtgcatcagatgctaaagcatatgacacagaggtacataatgtttgggccacacatgcctgtgtacccacagaccctaacccacaagaagtagtattgggaaatgtgacagaaaattttaatgtgtggaaaaatgacatggtagaacagatgcatgaagatataatcagcttatgggatcaaagcctaaagccatgtgtaaaattaaccccactctgtgttgctttaaattgcactaatgtgaatgatactaggacaaacaatagtagtagtagtgataaaaatgatgctaaaaccaatagtagtagtagttgggaaaggatggaaggagaagtaaaaaactgctctttcaatgttaccacaagaataagaaacaaggtgcagaaagaatatgcacttttttataagcttgatgtagtgccaatagagaaggataatgcaagctatacattgataaattgtaacacctcagtcattacacaggcctgtccaaaggtatcttttgaaccaattcccatacattattgtgccccggctggttttgcgattctaaagcgtaatgataagaagttcaatggaaaaggcccatgtacaaatgtcagcacagtacgatgtacacatggaattaggccagtagtgtcaactcaactactgttaaatggcagtctagcagaagaaggggtagtaattagatctgaaaatctcacgaacaatgttaaaaccataatagtacagctgaacaaatctgtaaaaattaattgtacaagacccaacaacaatacaagaaaaagtataaatataggaccagggagagcattttatgcaacaggagcaataataggaaatatgagacaagcacattgtaaccttaatggaacagaatggaagaacactttaagacaggtagttataagcttaagagagaaatttgggaagaagacaatagtcttcaaccaatcctcaggaggggatttagaaattataatgcacaattttaattgtggaggggaatttttctactgtgatacaacacagctgtttaatagtacttggctgcctaatgagactacagagtcaaataacattactggaggacctaatgacacactcacgctcccatgtagaataaaacaaattataaacagatggcaggaagtaggaaaagcaatgtatgcccctcccatcagtgggcaaattagatgctcatcaaatattacggggctgctattaacaagagatggtggtgaggagcagaatgacactgaggtctttagacctggaggaggagatatgagggacaattggagaagtgaattatataaatataaagtagtaagaattgagccatcaggagtagcacccaccaaggcaaagagaagagtggtgcaaagagacaaaagagcagtgggagcactaggagctatgttccttgggttcttgggagcagcaggaagcact

SEQ ID NO: 54 - protein, 490 aa, Human immunodeficiency virus type 1
SAAEEKWVTVYYGVPVWKEATTTLFCASDAKAYDTEVHNVWATHACVPTDPNPQEVVLGNVTENFNVWKNDMVEQMHEDIISLWDQSLKPCVKLTPLCVALNCTNVNDTRTNNSSSSDKNDAKTNSSSSWERMEGEVKNCSFNVTTRIRNKVQKEYALFYKLDVVPIEKDNASYTLINCNTSVITQACPKVSFEPIPIHYCAPAGFAILKRNDKKFNGKGPCTNVSTVRCTHGIRPVVSTQLLLNGSLAEEGVVIRSENLTNNVKTIIVQLNKSVKINCTRPNNNTRKSINIGPGRAFYATGAIIGNMRQAHCNLNGTEWKNTLRQVVISLREKFGKKTIVFNQSSGGDLEIIMHNFNCGGEFFYCDTTQLFNSTWLPNETTESNNITGGPNDTLTLPCRIKQIINRWQEVGKAMYAPPISGQIRCSSNITGLLLTRDGGEEQNDTEVFRPGGGDMRDNWRSELYKYKVVRIEPSGVAPTKAKRRVVQRD

SEQ ID NO: 55 - DNA, 1658 bp, Human immunodeficiency virus type 1
agaaagagcagaagacagtggcaatgaaagtgaaggggaccaggaagaattatcagcacttatggagatggggcaccatgctcctatggagatggggcaccatgctccttgggctattaatgatctgtaatgctatagaagaatcgtgggtcacagtccattatggagtacctgtgtggaaagaagcaaacaccactctgttttgtgcatcagatgctaaagcatatgatacagaggtacataatgtttgggccacacatgcctgtgtacccacaaaccccaacccacaagaagtagacttgggaaatgtgacagaaaattttaatgcatggaaaaatgacatggtagaacaaatgcatgaggatataattagtttatgggatcaaagcctaaagccatgtgtaaaattaactccactctgtgttactctacagtgcactgatttgaggaatgatactaataccactagtagtcctaataccactagtggtaactggatggataaaagggaaatgaaaaactgctctttcaatatcaccacaagcataagagataagctgcagaaaacatttgcacttttttataaacttgatatagtaccaataaatgaggacaaaaacagtagtaatattgataataccagttataggttgataagttgtaatacctcagtcattacacaggcctgtccaaaggtatcctttgagccaattcccatacattattgtgccccggctggttttgcgattctaaaatgtaaagatgaggagttcaatggaacaggaccatgtaaaaatgtcagtacagtacaatatacacatggaattaggccagtagtatcaactcaactgctgttaaatggcagtctagcagaacaaggggtagtacttagatctaaaaatatctcagacaatactaaaaccataatagtacagctaaaagaagctgtaacaattaagtgtacaagacccaacaacaatacaagaaaaagtatacatataggacctgggagagcattttatgcaacaggagacataataggagatataagacaagcccattgtaacatcagtgcaacaaagtggaatgacacgttacgtcagatagttgaaaaattacaaggatcatttaagaataaaacaataagcttcaagcgatcctcaggaggggatccagagattgtaatgcacagttttaattgtggaggggaatttttctattgtaattcaacaaaactgtttaatagtacttggtatagtaatgggactagtacttttgataatactactgaacgaacaaatgacactatcatactcccatgcagaataaaacaaattataaacatgtggcaggaagtaggaaaagcaatgtatgcccctcccatcccaggactaattaactgttcatcaaatattacaggactgctattaataagagatggtggtaataactatactgacaatactgagatcttcagacctggaggaggagacatgagggacaattggagaagtgaattatataaatataaagtagtaaaagttgaaccattaggtatagcacccaccaaggcaaagagaagagtggtacagagagaaaaaagagcagtgggaataggagcgtttttccttgggttcttgggagcagcaggaagcact

SEQ ID NO: 56 - protein, 490 aa, Human immunodeficiency virus type 1
NAIEESWVTVHYGVPVWKEANTTLFCASDAKAYDTEVHNVWATHACVPTNPNPQEVDLGNVTENFNAWKNDMVEQMHEDIISLWDQSLKPCVKLTPLCVTLQCTDLRNDTNTTSSPNTTSGNWMDKREMKNCSFNITTSIRDKLQKTFALFYKLDIVPINEDKNSSNIDNTSYRLISCNTSVITQACPKVSFEPIPIHYCAPAGFAILKCKDEEFNGTGPCKNVSTVQYTHGIRPVVSTQLLLNGSLAEQGVVLRSKNISDNTKTIIVQLKEAVTIKCTRPNNNTRKSIHIGPGRAFYATGDIIGDIRQAHCNISATKWNDTLRQIVEKLQGSFKNKTISFKRSSGGDPEIVMHSFNCGGEFFYCNSTKLFNSTWYSNGTSTFDNTTERTNDTIILPCRIKQIINMWQEVGKAMYAPPIPGLINCSSNITGLLLIRDGGNNYTDNTEIFRPGGGDMRDNWRSELYKYKVVKVEPLGIAPTKAKRRVVQRE

SEQ ID NO: 57 - DNA, 1655 bp, Human immunodeficiency virus type 1
agaaagagcagaagacagtggcaatgagagtgaaggagatcaggaagaattgtcagcgcttgtggacatggggcaccatgctccttgggatgttgatgatctgtagtactgcagaacaactgtgggtcacagtctattatggggtacctgtgtggaaagaagcaactaccactttattttgtgcatcagatgctaaagcatatgacacagaagcacataatgtttgggccacacatgcctgtgtacccacggaccctaacccacaagaagtagtaataaatgtgacagaaaattttaacatgtggaaaaatgacatggtagaacagatgcatgaggacataatcagtgtatgggaccagagtctaaagccatgtgtaaaattaaccccactctgtgttactttaaattgcactaattggaatggtactaataccaataatgctaatactaccagtagtcctaatattaccagtactactactgccaatatttatgagaaaagaatggaagaaggagaaatacaaaactgcactttcaatgtcaccacaagcataagggataaggtaaaagaagaatatgcacttttttatagatctgatgtaggccaaataggtaataatagtaataatacatatacattgataaattgtaattcctcagtcattacacaggctcgtccaaagatatcctttgaaccaattcccatacattattgtaccccggctggttttgcgattctaaaatgcaataataagaccttcaatggaacaggaccatgtaacaatgtcagcacagtacagtgtacacatggtattaggccagtagtatcaactcaattgttgttaaatggcagtctagcagaagatgagataatgattagatctgcaaatctctcggacaatactaaaaacataatagtacagctgaataaatctgtagaaattaattgtacaagacccaacaataatacaagaaaaagtataaatataggaccagggagagcattttatgcaacaggagatataataggaaacataagacatgcatattgtaccattaatgaaacaaaatggaatgaaactttaagacagatagctacaaaattacacaaacaatttaataaaacaataatctttgagcagtcctcaggaggagacccagaaattacaacgcacagttttaattgtggaggggaatttttctactgtaatacaacaccgctgtttaatagcacttgggttaagactcagaatgatactatagggtctaagactcagaatgctactacagggttaaatggcactatcatactcccatgcagaataaaacaaatcataaacagatggcaggaagtaggaagagcaatgtatgcccctcccatcaaaggaataattagatgttcatcaaatattacagggctgatattgacaagagatggtggtggtaatgagagcagggaaaatgagaccttcagacctggaggaggagatatgagggacaattggagaagtgaattatataaatataaagtagtaaaaattgagccaataggactggcacccaccaaggcaaagagaagagtggtgcagagagaaaaaagagcggtaacgctgggagctatgttccctgggttcttgggagcagcaggaagcact

SEQ ID NO: 58 - protein, 499 aa, Human immunodeficiency virus type 1
STAEQLWVTVYYGVPVWKEATTTLFCASDAKAYDTEAHNVWATHACVPTDPNPQEVVINVTENFNMWKNDMVEQMHEDIISVWDQSLKPCVKLTPLCVTLNCTNWNGTNTNNANTTSSPNITSTTTANIYEKRMEEGEIQNCTFNVTTSIRDKVKEEYALFYRSDVGQIGNNSNNTYTLINCNSSVITQARPKISFEPIPIHYCTPAGFAILKCNNKTFNGTGPCNNVSTVQCTHGIRPVVSTQLLLNGSLAEDEIMIRSANLSDNTKNIIVQLNKSVEINCTRPNNNTRKSINIGPGRAFYATGDIIGNIRHAYCTINETKWNETLRQIATKLHKQFNKTIIFEQSSGGDPEITTHSFNCGGEFFYCNTTPLFNSTWVKTQNDTIGSKTQNATTGLNGTIILPCRIKQIINRWQEVGRAMYAPPIKGIIRCSSNITGLILTRDGGGNESRENETFRPGGGDMRDNWRSELYKYKVVKIEPIGLAPTKAKRRVVQREKR

SEQ ID NO: 59 - DNA, 1637 bp, Human immunodeficiency virus type 1
agaaagagcagaagacagtggcaatgagagtgaaggggatcaggaagaattatcagcacttgtggacatggagcaacatgctcacgatgctccttgggatgttaatgatctgtagtgctgcagatcaattgtgggtcacagtctattatggggtacctgtgtggaaagaaacaaccaccactctattttgtgcatcagatgctaaagcatatgataaagaggtacataatgtttgggccacacatgcctgtgtacccacagaccccaacccacaagaagtaatattggaaaatgtgacagaaaattttaacgcgtggaaaaatgacatggtagaacagatgcatgaggatataattagtttatgggatcaaagcttaaagccatgtgtaaaattaaccccactctgtgttactttaaattgcactgatgctaatattactaataccaatgataatgagcccaatagtagtgtggtgaaactgatagagaaaggagaaataaaaaactgctctttcaatatcaccacaagcataagagataagatgcagaaagcatatgcacttttttataaacttgatgtagaaccaatagagaataatactactagctataggttgataagttgtaacacctcagtcattacacaagcctgtccaaaggtatcctttgagccaattcccatacatttttgtgccccggctggttttgcgattctaaagtgtaacaataagacattcgagggaaaaggaccatgtaaaaatatcagcacagtacaatgtacacatggaattaggccagtagtatcaactcaattgctgttaaatggcagtctggcagaagaagagatagtgattagatctgacaatttttcaaacagtgctaaaaccataatagtacagttgaatgcatctgtagaaattaatcgtacaagacccaacaacaatacgagaaaaggtatagttataggaccagggagaaaagttattgcaacagaaaaaataataggagatgtaagacaagcacattgtaacattagtataacaaaatggaataatactttaggccatatagttaataaattaagaaaacaatttggggaaaataaaacaatagtctttaagcaacactcaggaggggatccagaagttataatgcataattttaattgtgcaggggaatttttctactgtaatacaacaggactgtttaatagcacttggcattggaatggtacttggagtggtactgaaaggagaaatagcactgaaggaaatgacacacttacactcccatgcagaataaaacaaattataaacatgtggcaggaagtaggaaaagcaatgtatgcccctcccgttaacggacagattagatgtttatcaaatattacaggactgctattaacaagagatggtggtaataacaataacacaaacgacaccgaaaccttcagacctgaaggaggagatatgagggacaattggagaagtgaattatataaatataaagtagtaagaattgaaccattaggagtagcacccaccaaggcaaagagaagagtggtgcagagagaaaaaagagcactgggagtaggagcagctttgttccttgggttcttgggagcagcaggaagcact

SEQ ID NO: 60 - protein, 489 aa, Human immunodeficiency virus type 1
SAADQLWVTVYYGVPVWKETTTTLFCASDAKAYDKEVHNVWATHACVPTDPNPQEVILENVTENFNAWKNDMVEQMHEDIISLWDQSLKPCVKLTPLCVTLNCTDANITNTNDNEPNSSVVKLIEKGEIKNCSFNITTSIRDKMQKAYALFYKLDVEPIENNTTSYRLISCNTSVITQACPKVSFEPIPIHFCAPAGFAILKCNNKTFEGKGPCKNISTVQCTHGIRPVVSTQLLLNGSLAEEEIVIRSDNFSNSAKTIIVQLNASVEINRTRPNNNTRKGIVIGPGRKVIATEKIIGDVRQAHCNISITKWNNTLGHIVNKLRKQFGENKTIVFKQHSGGDPEVIMHNFNCAGEFFYCNTTGLFNSTWHWNGTWSGTERRNSTEGNDTLTLPCRIKQIINMWQEVGKAMYAPPVNGQIRCLSNITGLLLTRDGGNNNNTNDTETFRPEGGDMRDNWRSELYKYKVVRIEPLGVAPTKAKRRVVQREKR

SEQ ID NO: 61 - DNA, 1678 bp, Human immunodeficiency virus type 1
agaaagagcagaagacagtggaatgagagtgaaggggatcaggaagaattatcagcacttgtggatatggggcatcttgctccctgggatgttgatgatctgtagtgctgcagacaagttgtgggtcacagtctattatggggtacctgtgtggaaagaagcaaccaccactctattttgtgcatcagatgctaaagcatatagtgcagaggtacataatgtctgggccacacatgcctgtgtacccacagaccccgacccacaggaaatagtattggaaaatgtaacagaaaattttaacatgtggaaaaataacatggtagaacagatgcaggaggatataatcagtttatgggatcaaagcctaaagccatgtgtaaaattaacccctctctgtgtcactttaaaccgcactgatgaattgcggactactaataagactactaataagaccaatgatacagagacgaatactactaataccaccagctgggaaaaaggggaaatgaaaaactgctcttttgatgtcaccacaaacataagagataagtggcagagagaatatgcacttttttataagcttgatgtagtaccaatagataatgatggtaatggtaatagtagtaataatgccactgataataataatactaccaaatataccaactataggttgataagttgtaacacctcagttattacacaggcctgtccaaaggtatcctttgagccaattcccatacattattgtgccccagctggttttgcgattctaaagtgtaaagatgagaagttcagtggaacaggaccatgtaaaaatgtcagcacagtacaatgcacacatggaattaggccagtagtatcaactcaactgctgttgaatggcagtctagcaaaagaagagataataattagatctgaaaatctcacgaacaatgctaaaaccataatagtacagctgaatgaatctgtatcaattaattgtataagacccaacaataatacaagaagaggtatacctataggaccagggcaagcattttatgcaacaggggatataataggggatataagaaaagcacattgtatagttaacagtacacaatggaataacactttagcacaggtagccataaaattaaatgaacactttccaaataaaacaatagtctttaagcagtcctcaggaggggacccagaaattgtaatgcacagttttaattgtggaggggaatttttctactgtgattcaacaccactgtttaacaatacttggaatgaaacacattttaataatacttgggatagtattgaaaagggaaaaatcatactccaatgcagaataaaacaaattataaatatgtggcaggaagtaggaaaagcaatgtatgcccctcccatcagagggctgattaactgtacatcaaacattacagggctactattaacaagagatggtggcaagaaagagaatgagagtgatactatcgagatcttcagacctggaggaggagacatgaggaacaattggagaagtgaattatataaatataaagtagtaaaaattgaaccattaggagtagcacccaccaaggcaaaaagaagagtggtgcagagagaaaaaagagcagcgctaggagctttgttccttgggttcttgggagcagcaggaagcact

SEQ ID NO: 62 - protein, 508 aa, Human immunodeficiency virus type 1
SAADKLWVTVYYGVPVWKEATTTLFCASDAKAYSAEVHNVWATHACVPTDPDPQEIVLENVTENFNMWKNNMVEQMQEDIISLWDQSLKPCVKLTPLCVTLNRTDELRTTNKTTNKTNDTETNTTNTTSWEKGEMKNCSFDVTTNIRDKWQREYALFYKLDVVPIDNDGNGNSSNNATDNNNTTKYTNYRLISCNTSVITQACPKVSFEPIPIHYCAPAGFAILKCKDEKFSGTGPCKNVSTVQCTHGIRPVVSTQLLLNGSLAKEEIIIRSENLTNNAKTIIVQLNESVSINCIRPNNNTRRGIPIGPGQAFYATGDIIGDIRKAHCIVNSTQWNNTLAQVAIKLNEHFPNKTIVFKQSSGGDPEIVMHSFNCGGEFFYCDSTPLFNNTWNETHFNNTWDSIEKGKIILQCRIKQIINMWQEVGKAMYAPPIRGLINCTSNITGLLLTRDGGKKENESDTIEIFRPGGGDMRNNWRSELYKYKVVKIEPLGVAPTKAKRRVVQREKR

SEQ ID NO: 63 - DNA, 1595 bp, Human immunodeficiency virus type 1
agaaagagcagaagacagtggcaatgagagtgaaggagatcatgaagaactatcagaacttatggagagggggcatgatgctccttgggatattcatgatctgtagtgctacagaacaattgtgggtcacagtctattatggggtacctgtgtggaaagaagcaaataccactctattttgtgcatcagatgctaaagcatataagacagaggtacataatgtttgggccacacatgcctgtgtacccacagaccccaacccacaagaagtattattgccaaatatgacagaagattttaacatgtggaaaaataacatggtagaacagatgcatgaggatataatcagtttgtgggatcaaagcctaaagccatgtgtaaaattaaccccactctgtgttactttaaaatgcactgacttgaatactactaatactatcaatagtagtgacttgatggagaagggagaaataaagaactgctctttcaatatcaccacaaacataagagataagatgcagaaagactatgcgcttttttatagacttgatgtagtaccaatagataatgataatactagctataggttgataagttgtaacacctcagtcattacacaggcctgcccaaaggtatcttttgagccaattcccatacattgttgtgccccggctggttttgcgattctaaagtgtaaagataagaatttcaatggaacaggaacatgtaaaaatgtcagcacagtacagtgtacacatggaattagaccagtagtatcaactcaactgttgttaaatggtagtctggcagaagaagaggtagtaattagatctgccaatttcagtgacaatgctaaaaacataatagtacagctgaacgaaactgtagaaattaattgtacaagacccaacaacaatacaatgaaaagcatacatataggactagggagagcattttatacaacaggacaaataataggagatataagaaaagcacattgtagcattaatatgacaaaatggaataacaccttaatacaggtagctaaaaagttaggagaacaatttaagaataaaacaatagtctttaaccaatcctcaggaggggacacagaaattgtaatgcacagctttaattgtggaggggaatttttctactgcaatacaacacaactgtttaatggtagttggaatccaaatggtacttggaattatgctggggggtcaaacgacactatcacactcccatgcagaataaaacaaattataaatatgtggcaggaagtaggaaaagcaatgtatgcccctcccgtcaaaggacaaatcagatgtgtatcaaacattacagggttgatattaacaagagatggtggtaatggtggtaatggcacagacaacaccaccgagatctttaggcctgcaggaggaaatatgaaggacaattggagaagtgaattatataaatataaagtagtaagaattgaaccattaggagtagcacccactaaggcaaagagaagagtggtacaaagagaaaaaagagcagtgggaatgggagctctgttccttgggttcttgggagcagcaggaagcact

SEQ ID NO: 64 - protein, 479 aa, Human immunodeficiency virus type 1
SATEQLWVTVYYGVPVWKEANTTLFCASDAKAYKTEVHNVWATHACVPTDPNPQEVLLPNMTEDFNMWKNNMVEQMHEDIISLWDQSLKPCVKLTPLCVTLKCTDLNTTNTINSSDLMEKGEIKNCSFNITTNIRDKMQKDYALFYRLDVVPIDNDNTSYRLISCNTSVITQACPKVSFEPIPIHCCAPAGFAILKCKDKNFNGTGTCKNVSTVQCTHGIRPVVSTQLLLNGSLAEEEVVIRSANFSDNAKNIIVQLNETVEINCTRPNNNTMKSIHIGLGRAFYTTGQIIGDIRKAHCSINMTKWNNTLIQVAKKLGEQFKNKTIVFNQSSGGDTEIVMHSFNCGGEFFYCNTTQLFNGSWNPNGTWNYAGGSNDTITLPCRIKQIINMWQEVGKAMYAPPVKGQIRCVSNITGLILTRDGGNGGNGTDNTTEIFRPAGGNMKDNWRSELYKYKVVRIEPLGVAPTKAKRRVVQREKR

SEQ ID NO: 65 - DNA, 1592 bp, Human immunodeficiency virus type 1
agaaagagcagaagacagtggcaatgagagtgaaggggatcaggaggaattgtcagcacttatggagatggggcaccatgctccttgggatgttaatgatctgtagtgctacagaacaattgtgggtcacagtctattatggggtacctgtgtggaaagaagcaactaccaccctattttgtgcatcagatgctaaagcatatgatacagagagacataatgtttgggccacacatgcctgtgtacccacagacccctgcccacaagaagtaggattgggaaatgtgacagagtattttaacatgtggaaaaataacatggtagaacagatgcatgaggatataatcagtttatgggatcaaagcctaaagccatgtgtaaaattaacaccactctgtgttactttaaattgtaatgcgggaaagtttaattatacgaataatactgatacactgaaagaagaagtaggagaaataaaaaactgctctttcaatatcaccacaagcataagagataaggtaaagaaagaatatgcattttttaataaacttgatgtagtaccaatagataatgagaatgatagctataggttgataagttgtaacacctcagtcattactcaggcctgtccaaaggtatcatttgagccaattcctatacattattgtgccccagctggttttgcgattctaaggtgtaataataagacattcaatgggacaggaccatgtacaaatgtcagtacagtacaatgtacacatggaattaggccagtagtgtcaacccaactgctgttaaatggcagtctagcagaagaggaggtaatgattaggtctgagaacttcacgaacaatgctaaaaccataatagtacagctgaatgaatctgtagtaattaattgtacaagacccaacaacaatacaagaaaaagtatacacataggaccagggagagcattttatacaacaggagagataataggagatataagaaaagcacattgtaacattagtaaagcaaaatgggatagcactttaaaacaagtagttacaaaattaagagaacaatatggaaataaaacaatagcctttaagaactcctcaggaggggacccagaaattgtaatgcacagttttaattgtggaggggaatttttctactgtaatacaacaaagctatttaatagtacttggaataggacagaggtagatactattgaaggaaataccactataaatatcacactcccatgtagaataaaacaaattataaacatgtggcaggaagtaggaaaagcaatgtatgcccctcccatcagaggaccaattagctgcacatcaaatattacagggctgctgttaataagagatggtggtacagacaatagcacgaacgacaccgagatcttcagacctggaggaggagatatgagggacaattggagaagtgaattatacaaatataaagtagtaaaaattgaaccattaggaatagcacccaccaaggcaaagagaagagtggtgcagagagaaaaaagagcaataggaataggagctgtgttccttgggttcttgggagcagcaggaagcact

SEQ ID NO: 66 - protein, 478 aa, Human immunodeficiency virus type 1
SATEQLWVTVYYGVPVWKEATTTLFCASDAKAYDTERHNVWATHACVPTDPCPQEVGLGNVTEYFNMWKNNMVEQMHEDIISLWDQSLKPCVKLTPLCVTLNCNAGKFNYTNNTDTLKEEVGEIKNCSFNITTSIRDKVKKEYAFFNKLDVVPIDNENDSYRLISCNTSVITQACPKVSFEPIPIHYCAPAGFAILRCNNKTFNGTGPCTNVSTVQCTHGIRPVVSTQLLLNGSLAEEEVMIRSENFTNNAKTIIVQLNESVVINCTRPNNNTRKSIHIGPGRAFYTTGEIIGDIRKAHCNISKAKWDSTLKQVVTKLREQYGNKTIAFKNSSGGDPEIVMHSFNCGGEFFYCNTTKLFNSTWNRTEVDTIEGNTTINITLPCRIKQIINMWQEVGKAMYAPPIRGPISCTSNITGLLLIRDGGTDNSTNDTEIFRPGGGDMRDNWRSELYKYKVVKIEPLGIAPTKAKRRVVQREKR

SEQ ID NO: 67 - DNA, 1586 bp, Human immunodeficiency virus type 1
agaaagagcagaagacagtggcaatgaaagtgacggggatcatgaagaattatcagcacttatggagatggggcatcatgctccttgggatgttgatgatctatagtactgcagaacaacaattgtgggtcacagtctattatggggtacctgtgtggaaagaagcaactactactctattctgtgcatcagatgctaaagcatatgatacagaggtacataatgtttgggccacacatgcctgtgtacccacagaccccaacccacaagaagtagtattggggaatgtgacagaaaattttaacatgtggaaaaataacatggtagaacagatgcatgaggatataatcagtttatgggatcaaagcctaaaaccatgtgtaaaactaaccccactctgtgttactttaaattgcactgactgggataaaacgaattgcactaatgggggagatattactgctaatgaggaaaaaggagaactaaaaaattgctctttcaatatcaccacaaacataagagataagatacggaaagaatatgcacttttttataaattggatgtagtaccaatagataatgataatactagttataggttgataaattgtaacacctcagtcattacacaagcctgtccaaaggtatcctttgagccaattcccatacattattgtgccccggctggttttgcgattctaaagtgtaacaataagacgttcaatggaaaaggaccatgtaaaaatgtcagcacagtacaatgcacacatggaattaggccagtagtgtcaactcaactgctgttaaatggcagtttagcagaagaagaggtagtagttagatctgccaatttctcggacagtgccaaaaccatcatagtacaactaaatgaatctgtagtaattaattgtacaagacccaacaacaatacaagaaaaagcatacatataggaccagggagagcattttatgcaacaggagaaataataggagatataagacaagcacattgtaatcttagtctaacaaaatggaatcaaactttatatcaggtagttagaaaattaaaagaacaatttaagaataaaacaatagcctttaatcactcctcaggaggggacccagaaattgtaatgcacagttttaattgtggaggagaatttttctactgtaatacaacacaattatttaatagtacttggtatactaatggtacttggagtgatactggaagtaatgacacagtcacactcccatgcagaataaaacaaattataaacaggtggcaagaagtaggaaaagcaatgtatgcccctcccatcaaaggacaaattagatgctcatcaaatattacagggctgttattaacaagagatggtggtagtagcaaaaacgagaccgaggtcttcagacctggaggaggagatatgagggacaattggagaagtgaattatataaatataaagtagtaaaaattgaaccattaggagtagcacccaccagggcaaagagaagagtggtgcagagagaaaaaagaggaataggagctgtgttccttgggttcttgggagcagcaggaagcact

SEQ ID NO: 68 - protein, 478 aa, Human immunodeficiency virus type 1
STAEQQLWVTVYYGVPVWKEATTTLFCASDAKAYDTEVHNVWATHACVPTDPNPQEVVLGNVTENFNMWKNNMVEQMHEDIISLWDQSLKPCVKLTPLCVTLNCTDWDKTNCTNGGDITANEEKGELKNCSFNITTNIRDKIRKEYALFYKLDVVPIDNDNTSYRLINCNTSVITQACPKVSFEPIPIHYCAPAGFAILKCNNKTFNGKGPCKNVSTVQCTHGIRPVVSTQLLLNGSLAEEEVVVRSANFSDSAKTIIVQLNESVVINCTRPNNNTRKSIHIGPGRAFYATGEIIGDIRQAHCNLSLTKWNQTLYQVVRKLKEQFKNKTIAFNHSSGGDPEIVMHSFNCGGEFFYCNTTQLFNSTWYTNGTWSDTGSNDTVTLPCRIKQIINRWQEVGKAMYAPPIKGQIRCSSNITGLLLTRDGGSSKNETEVFRPGGGDMRDNWRSELYKYKVVKIEPLGVAPTRAKRRVVQREKR

SEQ ID NO: 69 - DNA, 1628 bp, Human immunodeficiency virus type 1
agaaagagcagaagacagtggcaatgagagcgaaggggaccaagaagaattggcagcagcacttatggaaatggggcacgatgctccttgggatgttaatgatctgtagtgctgcggaacaatggtgggtcacagtctattatggagtacctgtgtggaaggacgcaaataccactctattttgtgcatcagatgctaaagcatatgatacagaggcacataatgtctgggccacacatgcctgtgtacccacagatcccaacccacaagaaatagtattggaaaatgtgacagaagattttaacatgtggaaaaataacatggcagaccagatgcatgaggatataatcagtttatgggatcaaagcctaaagccatgtgtaaaattaactccgctctgtgttactttaaattgtactgattggaatggtaatactactagcaatagtactataaacaacaatactagtactaaggcagaaatgaaaaaatgctcttttaatatcaccacaagcataagagataaagtgacaaaggaatatgcactgttttatagagttgatgtagtaccaatagataaagaaaataataataccaattataccaattatagattaataaattgtaacacctcagtcattacacaagcctgtccaaagacatcctttgagccaattcctatacattattgtgccccagctggttttgcaattctaaagtgtaacaataagacattcacaggaaaaggactctgtacaagggttagcacattacaatgtacacatggaattagaccagtagtgtcaactcaactgctgttaaatggcagtctagcagaagaggaggtagtaattagatgtgaaaatatcacagacaatgctaaaaccataatagtacagctgaatgaatctgtagcaattaattgtacaagacctaataacaatacaagaaaaagtatacctataggaccagggagagcattttatgcaacaggagatatagtaggaaatataagacaagcacattgtaaccttagtggaacagagtgggaaaaaactttagggaaaatagttggggaattaagaaaaaattttgagaataagacaataatttttaatcaatcctcaggaggggacccagaaattgtatcgcacctttttaattgtggaggagaatttttctattgtaactcaacacaactgtttaatagtacttggaatactactgcaaaaattgatggttctgggaatgttactggaaaggtaaatagcactatcacactccaatgtaaaataagacaaattgtaaacctgtggcaggaagtaggaaaagcaatgtatgcccctcccatcagtggaataatttactgttcatcaaatattacagggctgatactgatgagagatggtggtaatgatagtagcacgaatggaaacgagaccttcagacctggagggggaaatatgaaagataattggagaagtgaattatataaatataaagtagtaaaaattgaaccattaggactagcacccaccaaggcaaagaggagagtggtgcaaagagaaaaaagagcagtaggaataggagctatgttccttgggttcttgggagcagcaggaagcact

SEQ ID NO: 70 - protein, 489 aa, Human immunodeficiency virus type 1
SAAEQWWVTVYYGVPVWKDANTTLFCASDAKAYDTEAHNVWATHACVPTDPNPQEIVLENVTEDFNMWKNNMADQMHEDIISLWDQSLKPCVKLTPLCVTLNCTDWNGNTTSNSTINNNTSTKAEMKKCSFNITTSIRDKVTKEYALFYRVDVVPIDKENNNTNYTNYRLINCNTSVITQACPKTSFEPIPIHYCAPAGFAILKCNNKTFTGKGLCTRVSTLQCTHGIRPVVSTQLLLNGSLAEEEVVIRCENITDNAKTIIVQLNESVAINCTRPNNNTRKSIPIGPGRAFYATGDIVGNIRQAHCNLSGTEWEKTLGKIVGELRKNFENKTIIFNQSSGGDPEIVSHLFNCGGEFFYCNSTQLFNSTWNTTAKIDGSGNVTGKVNSTITLQCKIRQIVNLWQEVGKAMYAPPISGIIYCSSNITGLILMRDGGNDSSTNGNETFRPGGGNMKDNWRSELYKYKVVKIEPLGLAPTKAKRRVVQREKR

SEQ ID NO: 71 - DNA, 1622 bp, Human immunodeficiency virus type 1
agaaagagcagaagacagtggcaatgagagtgaaggggatcaggaagaattggcggcgctggtggagatggggcaccatgctccttgggatgttaatgatctgtagtgctacagaacaattgtgggttacagtctattatggggtacctgtgtggaaagaagcaaccaccactctattttgtgcatcagatgctaaagcatataatacagaggtacgtaatgtatgggccacacatgcctgtgtgcccacaggccccaacccacaagaaatagtattggtaaatgtgacagaagattttaacatgtggaaaaatagcatggtagaacagatgcatgaggatataatcagtttatgggatcaaagcctaaaaccttgtgtaaaattaaccccactctgtgttactttaaactgcactgatttgaggaatactactaatagcactaatagtgacggggaaaagatggagagaggagaaataaaaaactgctctttcaatgttaccacaggcataagagataaggttcagagagaatatgcactcttttataaacttgatatagtaccaatagaggaaggtggggataataccagctgtagggataataccagctataggttgataagttgtaatacctcagtcattacacaagcctgtccaaaggtatcctttgagccaattcccatacattattgtgccccagctggttttgcgattctaaagtgtaataataagacgttcaatggaaaaggaccatgttcaaatgtcagcacagtacaatgtacacatggaattaggccagtagtgtcaactcaactgctgttaaacggcagtctagcagaaaaagaggtagtaattagatctgaaaatatcacggacaatactaaaaacataatagtacagttaaatgaaactgtagaaattaattgtacaagacccaacaacaatacaagaaaaagtatacatataggaccggggagagcatttcatgcaacaggagaaataataggaaatataagacaggcatattgtaacattagtggagcaaaatggaataacactttaaaacagatagtaaaaagattaaaagaaaaatttccgaataagataatagtctttaatcactcctcaggaggggacccagaaattgtaacacacagttttaattgtggaggggaatttttctactgtaattcaacaaacctgtttaatagtaattcaacacaactggataattggacttatactgaagggtcaaatgacactgttatcacgctcccatgcagaataaaacaaattgtaaatatgtggcaggaagtaggaaaagcaatgtatgctcctcccatcagaggacaaattagatgttcttcaaatattacagggctgatattaacaagagatggtggtaataagactgacaatgacaccaccgagaccttcagacctggaggaggaagcatgagggacaattggagaagtgaattatataaatataaaatagtaaaggttgaaccactaggagtagcacccaccaaggcaaagaggagagtggtgcagagagaaaaaagaacagtgggactgggagccttgtttcttgggttcttgggagcagcaggaagcact

SEQ ID NO: 72 - protein, 488 aa, Human immunodeficiency virus type 1
SATEQLWVTVYYGVPVWKEATTTLFCASDAKAYNTEVRNVWATHACVPTGPNPQEIVLVNVTEDFNMWKNSMVEQMHEDIISLWDQSLKPCVKLTPLCVTLNCTDLRNTTNSTNSDGEKMERGEIKNCSFNVTTGIRDKVQREYALFYKLDIVPIEEGGDNTSCRDNTSYRLISCNTSVITQACPKVSFEPIPIHYCAPAGFAILKCNNKTFNGKGPCSNVSTVQCTHGIRPVVSTQLLLNGSLAEKEVVIRSENITDNTKNIIVQLNETVEINCTRPNNNTRKSIHIGPGRAFHATGEIIGNIRQAYCNISGAKWNNTLKQIVKRLKEKFPNKIIVFNHSSGGDPEIVTHSFNCGGEFFYCNSTNLFNSNSTQLDNWTYTEGSNDTVITLPCRIKQIVNMWQEVGKAMYAPPIRGQIRCSSNITGLILTRDGGNKTDNDTTETFRPGGGSMRDNWRSELYKYKIVKVEPLGVAPTKAKRRVVQREKR

SEQ ID NO: 73 - DNA, 1622 bp, Human immunodeficiency virus type 1
agaaagagcagaagacagtggcaatgagagtgaaggggatcaggaagaattatcagcacttgtggagaggggacaacttgtggagagggggcatcatgctccttgggatattgatgatctgtagtgctacagaaaaattgtgggtcacagtctattatggggtacctgtgtggaaaaatgcaaacaccactctattttgtgcatcagatgctaaagcatatgatacagaggtacataatgtttgggccacacacgcctgtgtacccacagaccccagcccacgagaattaatattggaaaatgtgacagaagactttgacatatggaaaaataacatggtagaacagatgcaagaggatataatcagtttatgggatcaaagcctaaagccatgtgtaaaattaacccctctctgtgttactttagaatgcaagaatgccactaaaattagtaatagcagtgaaattggagaaatgaaaaactgctcttttaatgttaccacagacaggagagataaggtgaaaacagaatatgcacttttttataaccttgatataatacaaatagaggaggagaataccagcagctgcagtaataccagcagctacaggttgataagttgtaacacctcaacccttacacaggcctgtccaaagatatcctttgagccaattcccatacattattgtgccccggctggttttgcaattctaaagtgtaataataaaacattcgatggaaaaggatcatgtaaaaatgtcagcacagtacaatgtacacatggaattaagccagtagtatcaactcaactgctgctaaacggcagtctagcagaagaagaggtagtaattagatctgctaatctctcagacaatgctaaaaccataatagtacagctgaacatgtctgtacaaattaattgtacaagacccaacaacaatacaagaagaggtatacatttaggaccagggagagccttttatggaacagacataataggagatataagacaagcacattgtaacattagtggaaaacaatggaattacactttacaacagatagttaaaaaattcagaaaacaatttgagaatagcacagtgatctttaacagatcctcaggaggggacccagaaattgtaatgcacagttttaattgtggaggggaatttttctactgtaatacaacagaactgtttaatagtacttggaacagtagtcatcctttggatgatacttggcctcctttggataatacaagtgacactactatcacactcccatgcagaataaaacaaattataaacatgtggcaggaagtaggaaaagcaatgtatgcccctcccatcaaaggaccaattagatgtgaatcaaatattacagggctgctattaacaagagatggtggcgataccaataccactaacgggactgagaccttcagacctggaggaggagatatgagggacaattggagaagtgaattatataaatataaagtagtaagaattaaaccattaggaatagcacccaccaaggcacagagaagagtggtgcaaagagaaaaaagagcagcactaggagctatgttccttgggttcttgggagcagcaggaagcact

SEQ ID NO: 74 - protein, 483 aa, Human immunodeficiency virus type 1
SATEKLWVTVYYGVPVWKNANTTLFCASDAKAYDTEVHNVWATHACVPTDPSPRELILENVTEDFDIWKNNMVEQMQEDIISLWDQSLKPCVKLTPLCVTLECKNATKISNSSEIGEMKNCSFNVTTDRRDKVKTEYALFYNLDIIQIEEENTSSCSNTSSYRLISCNTSTLTQACPKISFEPIPIHYCAPAGFAILKCNNKTFDGKGSCKNVSTVQCTHGIKPVVSTQLLLNGSLAEEEVVIRSANLSDNAKTIIVQLNMSVQINCTRPNNNTRRGIHLGPGRAFYGTDIIGDIRQAHCNISGKQWNYTLQQIVKKFRKQFENSTVIFNRSSGGDPEIVMHSFNCGGEFFYCNTTELFNSTWNSSHPLDDTWPPLDNTSDTTITLPCRIKQIINMWQEVGKAMYAPPIKGPIRCESNITGLLLTRDGGDTNTTNGTETFRPGGGDMRDNWRSELYKYKVVRIKPLGIAPTKAQRRVVQREKR

SEQ ID NO: 75 - DNA, 1574 bp, Human immunodeficiency virus type 1
agaaagagcagaagacagtggcaatgacagtgatggggatcaggaggaattatcaatgcttgtggaaatggggcatgacgctccttgggatgttgatgatctgtagtgctgcacaattgtgggtcacagtctgttatggggtaccggtgtggaaagaagcaaccaccactctattttgtgcatcagatgctaaagcatatgacacagaggtacataatgtttgggccacacatgcctgtgtacccacagaccccaatccactagaattaaaattggataatgtgacagaaaattttaacatgtggaaaaataacatggtagaacaaatgcatgaggatataatcagtttatgggatcaaagcctaaagccatgtgtaaaattaaccccactctgtgttactttaaattgcactgactactcgaagaatggtactaataacactgctaataatgaaggagaaatgaaaaactgctcttttaatatcaccacaaacataagagataagatgcagaatgaatacgcacttttttataaacatgatatggtatcaatagataatagtagtactagctataggttgacaagttgtaacacctcagtcattacacaggcctgtccaaagataacctttgaaccaattcctatacattattgtaccccggctggttttgcgcttttaaagtgtaataataaaacgttcaatggaacaggaccatgtaaaaatgtcagcacagtacaatgtacacatggaattaggccagtagtttcaactcaactgctgttaaatggcagtctagcagaagaagaggtagtaattagatctgaaaatttcacgaacaatgcaaaaatcataatagtacagctaaatgaaactatacaaattaattgtacaagacccaacaacaatacaagaaaaagtatacatatagcaccagggagagcattttatgcaacaggagaaataataggagatataagacaagcacattgtaacattagcagagcaaaatggaacaacgctttaaaacagatagttgaaaaattaagagaacaatttaagaataaaacaatagaatttaagtcatcctcaggaggggacccagaaattgtaatgcacagtttcaattgtggaggggaatttttctactgtaattcaacaaaactgtttaatagtatttggtatccgaatggtactgaagggtcaaataacactgaaggaaatgacccaatcacactcccatgcagaataagacaaattgtaaacagatggcaggaagtaggaaaagcaatgtatgcccctcccatcaggggaccaattagatgttcatcaaatattacagggctgctattaacaagagatggtggtgctaataatactgataatgagaccttcagacctggaggaggagatatgagggacaattggagaagtgaattatataaatataaagtagtaagaattgaaccattaggaatagcgcccactacggcaaggagaagagtggtgcaaagagaaaaaagggcagccataggagctatgatccttgggttcttgggagcagcaggaagcact

SEQ ID NO: 76 - protein, 473 aa, Human immunodeficiency virus type 1
SAAQLWVTVCYGVPVWKEATTTLFCASDAKAYDTEVHNVWATHACVPTDPNPLELKLDNVTENFNMWKNNMVEQMHEDIISLWDQSLKPCVKLTPLCVTLNCTDYSKNGTNNTANNEGEMKNCSFNITTNIRDKMQNEYALFYKHDMVSIDNSSTSYRLTSCNTSVITQACPKITFEPIPIHYCTPAGFALLKCNNKTFNGTGPCKNVSTVQCTHGIRPVVSTQLLLNGSLAEEEVVIRSENFTNNAKIIIVQLNETIQINCTRPNNNTRKSIHIAPGRAFYATGEIIGDIRQAHCNISRAKWNNALKQIVEKLREQFKNKTIEFKSSSGGDPEIVMHSFNCGGEFFYCNSTKLFNSIWYPNGTEGSNNTEGNDPITLPCRIRQIVNRWQEVGKAMYAPPIRGPIRCSSNITGLLLTRDGGANNTDNETFRPGGGDMRDNWRSELYKYKVVRIEPLGIAPTTARRRVVQREKR

SEQ ID NO: 77 - DNA, 1610 bp, Human immunodeficiency virus type 1
agaaagagcagaagacagtggcaatgaaagtgaaggggatcaggaagaattgtcagcgctggtggatagggggcatcttgctccttggaatgttgatgatctgtagtgctgcagaacaattgtgggtcacagtctattatggggtacctgtgtggaaagaagcaaacatcactctattttgtgcatcagatgctaagggatatgatacagaagcacataatgtctgggccacacatgcctgtgtacccacagaccccaacccacaagaggtagcattgacaaatgtgacagaaaactttaacatgtggaaaaataacatggtagaacaaatgcatgaggatataattagtttatgggatcaaagcttaaagccatgtgtaaaattaaccccactctgtgttactttagattgcaatgatactaatataaatgtaactagcaaaaatgagacaatgatggagcaaggagaagcaaaaaactgctctttcaatatcaccacaaatttaagagataaggtgcagaaagaatattcagttttttataaacttgatgtagtaccaatagaagaggagaaaaataatagtattaacaatagatataggttgataagttgtaacacctcagtcattacacaagcctgtccaaagatatcctttgaaccaattcccatacattattgtgccccggctggttttgcgattctgaagtgtaacaataagacattcagtggaaaaggaccatgcacaaatgtcagcacagtacaatgcacacatggaattaggccagtagtatcaactcaactgctgttaaatggcagcctagcagaaggagagatagtaattagatctgacaatttcacagacaatacaaaaaccataatagtacagctgaatacatctgtagcaattaattgtacaagacccaacaacaatacaagaagaagtataactataggaccagggagagcattttatgcaacagacataataggagatataagacaagcacattgtaacattagtagaacacaatggaataacactttaaaacaggtagctagaaaattaagtgaacaatttaatgcaacaatagtttttaataaatcctcaggaggggacccagaaattgtaatgcacagttttaattgtggaggggaatttttctactgtaatacaacacaactgtttaatagtatttggtgtcctaataatactggagagtcaaatagcactaacaatgagacaatcatactcccatgcagattaaaacaatttataaacatgtggcaggaggtaggaaaagcaatgtatgcccctcccatcagaggatacattaactgttcatcaaatattacggggctgctattaacaagagatggtggtggtgataataaaaacacgagtccagagaacaagacagagaccttcagacctggaggaggaaatatgaaggacaattggagaagtgaactgtataaatataaagtagtaagaattgaaccattaggagtagcacccaccaaggcaaagagaagagtggtgcagagagaaaaaagagcagtgggaataggagctatgttccttgggttcttgggagcagcaggaagcact

SEQ ID NO: 78 - protein, 484 aa, Human immunodeficiency virus type 1
SAAEQLWVTVYYGVPVWKEANITLFCASDAKGYDTEAHNVWATHACVPTDPNPQEVALTNVTENFNMWKNNMVEQMHEDIISLWDQSLKPCVKLTPLCVTLDCNDTNINVTSKNETMMEQGEAKNCSFNITTNLRDKVQKEYSVFYKLDVVPIEEEKNNSINNRYRLISCNTSVITQACPKISFEPIPIHYCAPAGFAILKCNNKTFSGKGPCTNVSTVQCTHGIRPVVSTQLLLNGSLAEGEIVIRSDNFTDNTKTIIVQLNTSVAINCTRPNNNTRRSITIGPGRAFYATDIIGDIRQAHCNISRTQWNNTLKQVARKLSEQFNATIVFNKSSGGDPEIVMHSFNCGGEFFYCNTTQLFNSIWCPNNTGESNSTNNETIILPCRLKQFINMWQEVGKAMYAPPIRGYINCSSNITGLLLTRDGGGDNKNTSPENKTETFRPGGGNMKDNWRSELYKYKVVRIEPLGVAPTKAKRRVVQREKR

SEQ ID NO: 79 - DNA, 1607 bp, Human immunodeficiency virus type 1
agaaagagcagaagacagtggcaatgaaagcgaaggggaccaggaagaattatcagcacttgtggagatggggtaccatgctccttgggatgttgatgatctgtagtgctacagagcaattgtgggtaacagtctattatggggtacctgtgtggaaagaagcaaaaaccactctattttgtgcatcagatgctaaagcatatgatacagagatgcataatgtttgggccacacatgcctgtgtacccacagaccccaacccacaagaaatagtattggaaaatgtgacagaaaattttaacatgtggaaaaataacatggtagatcagatgcaggaggatgtaatcagtctatgggatcaaagcctaaagccatgtgtaaaattaaccccactctgtgttactttaaattgcaatgatacattgaggaatgataatagcactaagaataatagtagtactggttgggaaaagatggagaaaggagaaataaaaaattgctctttcagtgccaccacaaccgtgaaagataagacacagaaacaatatgcacttttttataatcttgatatagtacatacaaatgatggtggtaatagtagctatatgttaagaagttgtaacacctcagtcattacacaggcctgtccaaaggtatcatttgagccaattcccatacattattgtgccccggctggttttgcgattctaaagtgtaatgataagaagttcaatgggacaggaccatgtaaaaatgtcagcacagtacaatgtacacatggaattaggccagtagtgtcaactcaactgctattaaatggcagtctagcagaagaagaggtagtaattagatctagcaatttaacggacaatactaaaaccataatagtacagctgaaggaatctgtaaaaattaattgtacaagacccaacaacaatacaagaaaaagtatatctataggaccagggagcgcattttatgcaacaggagacataataggagatataagacaagcacattgcaaccttagtaaaacagaatggggggaaactttaagacagatagctacaaaattaagagaacaatttaataataaaacaataatctttaatagctcctcaggaggggacccagaaattgtaatgcacagttttaattgtggaggggaatttttctactgtaatacaacaaaactgtttaatggtacttggaatggtacttggaagaatggtacttggagtacaaatgatactgaaaatgatactatcatactcccatgtagaataaaacaaattataaacatgtggcaggaagtaggaaaagcaatgtgtgcccctcccatcagaggacaaattaactgctcatcacagattacagggctgctattaacaagagatggtggagataaccctaaaaatgaaaccttcagacctggaggaggagatatgagggacaattggagaagtgaattatataaatataaagtagtaaaaattgaaccattaggaatagcacctaccagggcaaagagaagagtggtgcagagagaaaaaagagcagcaataggagctatgatccttgggttcttgggagcagcaggaagcact

SEQ ID NO: 80 - protein, 484 aa, Human immunodeficiency virus type 1
SATEQLWVTVYYGVPVWKEAKTTLFCASDAKAYDTEMHNVWATHACVPTDPNPQEIVLENVTENFNMWKNNMVDQMQEDVISLWDQSLKPCVKLTPLCVTLNCNDTLRNDNSTKNNSSTGWEKMEKGEIKNCSFSATTTVKDKTQKQYALFYNLDIVHTNDGGNSSYMLRSCNTSVITQACPKVSFEPIPIHYCAPAGFAILKCNDKKFNGTGPCKNVSTVQCTHGIRPVVSTQLLLNGSLAEEEVVIRSSNLTDNTKTIIVQLKESVKINCTRPNNNTRKSISIGPGSAFYATGDIIGDIRQAHCNLSKTEWGETLRQIATKLREQFNNKTIIFNSSSGGDPEIVMHSFNCGGEFFYCNTTKLFNGTWNGTWKNGTWSTNDTENDTIILPCRIKQIINMWQEVGKAMCAPPIRGQINCSSQITGLLLTRDGGDNPKNETFRPGGGDMRDNWRSELYKYKVVKIEPLGIAPTRAKRRVVQREKR

SEQ ID NO: 81 - DNA, 1580 bp, Human immunodeficiency virus type 1
agaaagagcagaagacagtggcaatgagagtgagggggattatgaggaattatcagcacttgtggagatggggcatgacgctccttgggatgttaatgatcagtagtgctaatgaacaattgtgggtcacagtctgttatggggtacctgtgtggaaagaagcaaccactactttattttgtgcatcagatgctaaagcatatgctgcagagaaacataatgtttgggccacacatgcctgtgtacccacagaccccaacccacaagaagtagtaataaatgtgacagaaaattttaacatgtggaaaaataacatggtagagcagatgcatgaagatgtaactagtttatgggaccaaagcctaaagccatgtgtaaaattaacccctctctgtgttactttaaattgcactgactatgaggggaataatatcactagtgggaataagacaggagaaataaaaaactgctctttcgagatcaccacaaacatgagagataagatacagaaaacatatgcacttttttatagacttgatgtagaaccaataaatgatgataatgttacttataggttgataagctgtaatacctcagtcattacacaagcctgtccaaaggtaacctttgagccaattcccatacattattgtgccccggctggctttgcgattgtaaagtgtaacaataaaacgttcaatggaacaggaccatgtaaaaatgttagcacagtacaatgtacacatggaattaggccagtagtatcaactcaactgctgttaaatggcagtctagcggaagaagagacaatgattagatctgagaatttctcggacaatgctaaaatcataatagtacagctgaataaatctgtaaaaattaattgtacaagacccaacaataatacaataaaaggtatacatataggaccagggagagcattttatacaacaggacaaataataggagacataagacaagcatattgtaccattaataaaacagaatggaataacactttatcacagatagctaaaaaattaagtagacaatttgagaataaaacaatagcctttaggccaccctcaggaggggacccagaaattgtaatgcacagttttaattgtggaggggaatttttctattgtaatacaacacaactgtttaatagtaattggactactaatggagagtcaaattacacaacgggaaacaatgagacaattatcacactcccatgcagaataaaacaatttataaacatgtggcaggaagtaggaaaagcaatgtatgcccctcccattagtggaataattaattgcttatcaaatattacagggctgctattaacaagagatggtggtaatagtagcagcgccaacagcaccgagatcttcagacctggaggaggggatatgagggataattggagaagtgaactatataaatataaagtagtacaaattgaaccattaggattagcacccaccaaggcaaagagaagagtggtgcagagagaaaaaagagcagtgggactaggagctgtgttccttgggttcttgggagcagcaggaagcact

SEQ ID NO: 82 - protein, 474 aa, Human immunodeficiency virus type 1
SANEQLWVTVCYGVPVWKEATTTLFCASDAKAYAAEKHNVWATHACVPTDPNPQEVVINVTENFNMWKNNMVEQMHEDVTSLWDQSLKPCVKLTPLCVTLNCTDYEGNNITSGNKTGEIKNCSFEITTNMRDKIQKTYALFYRLDVEPINDDNVTYRLISCNTSVITQACPKVTFEPIPIHYCAPAGFAIVKCNNKTFNGTGPCKNVSTVQCTHGIRPVVSTQLLLNGSLAEEETMIRSENFSDNAKIIIVQLNKSVKINCTRPNNNTIKGIHIGPGRAFYTTGQIIGDIRQAYCTINKTEWNNTLSQIAKKLSRQFENKTIAFRPPSGGDPEIVMHSFNCGGEFFYCNTTQLFNSNWTTNGESNYTTGNNETIITLPCRIKQFINMWQEVGKAMYAPPISGIINCLSNITGLLLTRDGGNSSSANSTEIFRPGGGDMRDNWRSELYKYKVVQIEPLGLAPTKAKRRVVQREKR

SEQ ID NO: 83 - DNA, 1643 bp, Human immunodeficiency virus type 1
agaaagagcagaagacagtggcaatgagagtgaaggagatcaggaagaattgtcagaaattctggaaatggggcaccttgctccttgggatgttaatgatgatctgtagagctgcagaggattcgtgggtcacagtctattatggggtacctgtgtggaaggaagcaaccaccactctattttgtgcatcagatgctaaagcatatgacacagaggtacataatgtttgggccacacatgcctgtgtacccacagaccctaacccacaagaagtagtattggaaaatgtgacagaaaatttcaatgcgtggaaaaataatatggtagaacagatgcatgaggatataatcagtttatgggatcaaagcctaaagccatgtgtaaaattaacccctctttgtgttactctaaattgcactgatgtgagaaataatgctaccaatactactaataccaatagtactaatacctatagtactaacatagaaaagatgaaagaaggagaaataaaaaactgctctttcaataccaccccaagcataacagacaagatgcagaaggcatatgcattgttttataagcttgatatagtacagataaataatgataataaagataataccagctatagattgataagttgtaatacctcagtcattacacaggcctgtccaaaggcatcctttgagccaattcccatacattattgtgccccagctggttttgcgattctaaagtgtaatgataagaagttcaatggaacaggaccatgcaaaaatgtcagcacagtacaatgtacacatggaattaggccagtagtatcaacccaactgctgttaaatggcagtctagcagaagaagaggtagtaattagatctgaaaatttcacaaacaatgctaaaaccataatagtacagctgaatgagactgtacatattaattgtacaagacccaacaacaatacaagaaaaagtataggtataggaccagggagaacattttttgcaacaggggaaataataggagacataagacaagcacattgtaacattagtagaaacaactggaataaaactttagaaagggtagttaaaaaattaagagaacaatttgggaacaacaaaacaattgtttttaatcaatcctcaggaggggacccagaaattgtgatgcacagttttaattgtagaggagaatttttctactgtaatacaacacaactgtttaatagtacttggaatgctaatagtacttggaatgctaacgagaatactactggaatgccaagtgacaatatcacactcccctgcagaataaaacaagttataaacatgtggcaggaagtaggaaaagcaatgtatgcccctcccattaaaggaccaattaagtgttcatcaaatattacaggactgctattaacaagggatggtggtgtgaacaattctgaaactgagaccttcagacctggaggaggagatatgaggaacaattggagatgtggattatataaatataaagtagtaaaaattgagccattaggagtagcacccaccagggcaaagagaagagtggtgcaaagagaaaaaagagcagtgggactaggagctatgttccttgggttcttgggagcagcaggaagcact

SEQ ID NO: 84 - protein, 494 aa, Human immunodeficiency virus type 1
RAAEDSWVTVYYGVPVWKEATTTLFCASDAKAYDTEVHNVWATHACVPTDPNPQEVVLENVTENFNAWKNNMVEQMHEDIISLWDQSLKPCVKLTPLCVTLNCTDVRNNATNTTNTNSTNTYSTNIEKMKEGEIKNCSFNTTPSITDKMQKAYALFYKLDIVQINNDNKDNTSYRLISCNTSVITQACPKASFEPIPIHYCAPAGFAILKCNDKKFNGTGPCKNVSTVQCTHGIRPVVSTQLLLNGSLAEEEVVIRSENFTNNAKTIIVQLNETVHINCTRPNNNTRKSIGIGPGRTFFATGEIIGDIRQAHCNISRNNWNKTLERVVKKLREQFGNNKTIVFNQSSGGDPEIVMHSFNCRGEFFYCNTTQLFNSTWNANSTWNANENTTGMPSDNITLPCRIKQVINMWQEVGKAMYAPPIKGPIKCSSNITGLLLTRDGGVNNSETETFRPGGGDMRNNWRCGLYKYKVVKIEPLGVAPTRAKRRVVQREKR

SEQ ID NO: 85 - DNA, 1603 bp, Human immunodeficiency virus type 1
agaaagagcagagacagtggcaatgaaagtgaaggggatcaggaagaattatcagcacttgtggggatggggcatgatgctccttgggatgttaatgatctgtagtgctacagaaaacctgtgggtcacagtatattgtggggtacctgtgtggaaagaagcagaaaccagtctattttgtgcatcagatgctaacacatataatacagaggctcataatgtttgggccactcatgcctgtgtacccacggaccccaacccacaagaaatatatttggaaaatgtgacagaaaattttaacatgtggaaaaataacatggtagaacagatgcatgaggatatagtaagtttatgggatgaaagcctaaagccatgtgtaaaaataaccccactctgtgtcactctaaattgcactgatttggaaaatggcactagtagcaataatagtagctatcaaaggggggaagaaggagaaataaagaactgctctttcaatatcaccacaagattaagagaaaaggtacagaaagaatatgcacttttttataaacttgatataatagcaatggataataaaactaatgctaccagatataggttgataagttgtaacacctcaaccattacacaggcctgtccaaaagtatcctttgagccaattcccatacattattgtgccccagctggttttgcgcttttcaagtgtaatgataagaagttcaatggatcaggaacatgtaacaatgtcagcacagtacaatgtacacatggaattaggccagtagtatcaactcagctgttgctaaatggcagtctagcagaagaagaggtagtaattagatctgaaaatttcacaaacagtgctaaaaccataatagtacagctaaaagaacctgtaaaaattaattgtacaagacccaacaataatacaagaagaagtatacatataggaccaggaaaagcattttatgcaacaggagaaataataggagatataggacaagcacattgtaacattagtggacaagaatggaataaaactttaattcagatagttaaaaaattgagagaacaatttgggaataagacgataaactttactaaaccagcaggaggggacccagagattgtaatgcacagttttaattgtggaggggaatttttctactgtgatacaacacgactgtttaatagggcttggaataatactgaagagttaaatagtactactggagagtcaaataacactatcaccctcccatgcagaataaaacaaattataaacatgtggcaggaagtaggaaaagcaatgtatgcccctcccatccaaggaacaattagatgttcatcaaatattacagggctgctactagcaagagatggtggcagtaacaatgagactaatactactgaaatcttcagacctgcaggaggagatatgagggacaattggagaagtgaattatataaatataaagtagtaaaaattgaaccattaggagtagcacccaccagggcaaagagaagagtggtgcaaagagaaagaagagcaataggaataggagctgtgttccttgggttcttgggagcagcaggaagcact

SEQ ID NO: 86 - protein, 482 aa, Human immunodeficiency virus type 1
SATENLWVTVYCGVPVWKEAETSLFCASDANTYNTEAHNVWATHACVPTDPNPQEIYLENVTENFNMWKNNMVEQMHEDIVSLWDESLKPCVKITPLCVTLNCTDLENGTSSNNSSYQRGEEGEIKNCSFNITTRLREKVQKEYALFYKLDIIAMDNKTNATRYRLISCNTSTITQACPKVSFEPIPIHYCAPAGFALFKCNDKKFNGSGTCNNVSTVQCTHGIRPVVSTQLLLNGSLAEEEVVIRSENFTNSAKTIIVQLKEPVKINCTRPNNNTRRSIHIGPGKAFYATGEIIGDIGQAHCNISGQEWNKTLIQIVKKLREQFGNKTINFTKPAGGDPEIVMHSFNCGGEFFYCDTTRLFNRAWNNTEELNSTTGESNNTITLPCRIKQIINMWQEVGKAMYAPPIQGTIRCSSNITGLLLARDGGSNNETNTTEIFRPAGGDMRDNWRSELYKYKVVKIEPLGVAPTRAKRRVVQRERR

SEQ ID NO: 87 - DNA, 1604 bp, Human immunodeficiency virus type 1
agaaagagcagaagacagtggcaatgagagtgaaggggatcaggaagaattgtcagcacttatggagatggggcatcatgctccttggaatgttaatgatctgtagtgctgcaagcctgtgggtcacagtctattatggggtacctgtgtggaaagatgcaaacaccactctattttgtgcatcagatgctaaagcatatgatacagaggtacataatgtgtgggccacacatgcctgtgtacccacagaccccaacccacaagaagtagtattggaaaatgtgacagaaaattttaatatgtggaaaaataacatggtagaacagatgcatgaggacataattagtttatgggaccaaagcctaaagccatgtgtaaaactaaccccactctgtgttactttaaattgcactgagttgatgttgaatactactaccaatagtactactaccaatagtaccagtagtcctcctaccagtagtggattgacaaactgctctttcaatatcgccacagatctaagagataaggtgcagaaagaatatgctcttttttctacacttgatgtagtatcaataggtaataacagctctaggctgataagttgtaacacctcaatccttacacaggcctgtccaaaggtatcctttgagccaattcccatacattattgtgccccggctggttttgcaattctaaagtgtaacaataagacattcaatggaaaaggactatgtaacaatgtcagcacaatacaatgtacacatggaattaagccagtagtatcaactcaattactgttaaatggcagtctagcagagaaagacatagtaattagatctgacaatttttcaaacaatgctaagaccataatagtacagctgaagaagcctgtatacatcaattgtacaagacccaacaacaatacgagaaaaggtatacacatagcaccagggagagcattttatacaacaggacagataataggagacataaggaaagcatattgtgaaattagtggaaaaagctggaataacactttagaacagatagctacaaaattaagagaacaatttgggagtaataaaacaatagtctttaatcaatcctcgggaggggacccagaaattgtaatgcacagttttaattgtagaggagaatttttctattgtaattcaacacaattgtttaatagtacttggccgggtaacggtcctagcaataatactactgggaatggtactgatactgttatcatccttccatgcagaataaaacaaatcataaacatgtggcaggaagtaggaagagcaatgtgtgcccctcccatcgcaggacaaattaactgtacaacaaaaattacagggctgttattaacaagagatggtgggaatagcaatgagaccaaagagactgaaatctttagacctggaggaggagatatgagggacaattggagaagtgaattatacaaatataaagtagtaaaaattgaaccattaggagtagcacccaccgaggcaaggagaagagtggtgcaacgagaaaagagcagtgggactaggagctatgttccttgggttcttgggagcagcagaagcact

SEQ ID NO: 88 - protein, 483 aa, Human immunodeficiency virus type 1
SAASLWVTVYYGVPVWKDANTTLFCASDAKAYDTEVHNVWATHACVPTDPNPQEVVLENVTENFNMWKNNMVEQMHEDIISLWDQSLKPCVKLTPLCVTLNCTELMLNTTTNSTTTNSTSSPPTSSGLTNCSFNIATDLRDKVQKEYALFSTLDVVSIGNNSSRLISCNTSILTQACPKVSFEPIPIHYCAPAGFAILKCNNKTFNGKGLCNNVSTIQCTHGIKPVVSTQLLLNGSLAEKDIVIRSDNFSNNAKTIIVQLKKPVYINCTRPNNNTRKGIHIAPGRAFYTTGQIIGDIRKAYCEISGKSWNNTLEQIATKLREQFGSNKTIVFNQSSGGDPEIVMHSFNCRGEFFYCNSTQLFNSTWPGNGPSNNTTGNGTDTVIILPCRIKQIINMWQEVGRAMCAPPIAGQINCTTKITGLLLTRDGGNSNETKETEIFRPGGGDMRDNWRSELYKYKVVKIEPLGVAPTEARRRVVQREKS

SEQ ID NO: 89 - DNA, 1666 bp, Human immunodeficiency virus type 1
agaaagagcagagacagtggcaatgagagcgaaggagaccaggaagaattgtcagcactcgtggggatggggaaccatgctcctgtggagatggggcatcatgctccttgggatgttaatgatctgtagtgctaaagaaaatttgtgggtcacagtctattatggggtacctgtgtggaaagaagcatccaccactctattttgtgcatcagatgctaaagcatatgatacagaggtacataatgtttgggccacacatgcctgtgtacccacagaccccagcccacaagaagtagtattgggaaatgtgacagaatgttttaacatgtggaataacaacatggtagaacagatgcatgaggatataatcagtttatgggaccaaagtctaaaaccctgtgtaaaattaaccccactctgtgttaccttaagttgcagtgacgttaatattaccaatattatcaataacactattgctaaaaataatagtttaagaatggaaacaggagacataaaaaactgctctttcaatatcaccacaaacataagagataagatgcaaacagaatatgcacttttttataaatttgatgtagtgccaatatatgatagcaatgatgatagcaatattactagaaatgatagttataggataataagttgtaatacctcagtcattacacaggcctgtccaaaggtaacctttgagccaattcccatacattattgtgccccggctggttttgcgattctaaagtgtaacaataagacattcaatggaaaaggaccatgtacaaatgtcagcacagtacaatgtacacatggaattaggccagtagtgtcaactcaactactgttaaatggcagtctagcagaaaaggagatagtgattagatctgacaatttctcggacaatgctaaaactataatagtacagttaaatggaactgttcaaattaattgttcaagacccggcaacaatacaagaaaaagtatacatataggaccagggagtgcattttatgcaacaggagacataataggagatataagaaaagcacattgtaacattagtaaaacagactggaataacactttaggaaagatagcaaaaaaattaagagaacaatttggggaaaataaaacaatagagtttgagaaatccacaggaggggacccagaagttatgatgcatacttttaattgtggaggggaatttttctactgtaattcaacaccgctgtttaatggtagtacttttaataatacttggacacctttgaatagtagtgctaaagggccaaatgacactctcatactccaatgtagaataaaacaaatcataaacatgtggcaggaagtaggaaaagcaatgtatgcccctcccatcagaggatacattaattgttcatcaaatattacagggctgctattgacaagagatggtggtaataatactggtaatgatagcaataccgagaccttcagacctacaggaggaaatatgaaggacaattggaggagtgaattatataaatataaagtagtacaaattgaaccattaggagtagcacccaccagggcaaaaagaagagtggtgcagagagaaaaaagagcagcgctgggggctatgttccttgggttcttgggagcagcaggaagcact

SEQ ID NO: 90 - protein, 496 aa, Human immunodeficiency virus type 1
SAKENLWVTVYYGVPVWKEASTTLFCASDAKAYDTEVHNVWATHACVPTDPSPQEVVLGNVTECFNMWNNNMVEQMHEDIISLWDQSLKPCVKLTPLCVTLSCSDVNITNIINNTIAKNNSLRMETGDIKNCSFNITTNIRDKMQTEYALFYKFDVVPIYDSNDDSNITRNDSYRIISCNTSVITQACPKVTFEPIPIHYCAPAGFAILKCNNKTFNGKGPCTNVSTVQCTHGIRPVVSTQLLLNGSLAEKEIVIRSDNFSDNAKTIIVQLNGTVQINCSRPGNNTRKSIHIGPGSAFYATGDIIGDIRKAHCNISKTDWNNTLGKIAKKLREQFGENKTIEFEKSTGGDPEVMMHTFNCGGEFFYCNSTPLFNGSTFNNTWTPLNSSAKGPNDTLILQCRIKQIINMWQEVGKAMYAPPIRGYINCSSNITGLLLTRDGGNNTGNDSNTETFRPTGGNMKDNWRSELYKYKVVQIEPLGVAPTRAKRRVVQREKR

SEQ ID NO: 91 - DNA, 1559 bp, Human immunodeficiency virus type 1
agaaagagcagaagacagtggcaatgagagtgaaggagatcaggaagaactatcagcacttgtggagatggggcatcatgctctttgggatattaatgatctgtagtgctgaagaaaagtgggtcacagtctattatggggtacctgtgtggaaagaagcaaagaccactctattttgtgcatcagatgctaaagcatatgatacagaggcacataatgtttgggccacacatgcctgtgtacccacagaccccaacccacaagaagtagtattggagaatgtgacagaaaattttaacatgtggaaaaatgacatggtagagcagatgcatgaggatgtaatcagtttatgggatcaaagcctaaagccatgtgtagaattaacgccactctgtgttactctaaattgcactaatctaaattgcactaacaacactagtagcgaaataaaaaactgttctttctatgtcaccacaagcatggaaggtaaggtgaaaaaacatgcaacgttttatagccttgatatagtacgaacaacagagagtaatatcagctataggttgataagttgtaacacctcagtcattacacaggcctgtccaaaaatatcctttgaaccaattcccatacattattgtgccccggctggttttgcgatcctaaagtgtaacaataagacattcaatggaacaggaccatgtacaaatgtcagcacagtgcaatgtacacatggaattaagccagtagtatcaactcaactactgttaaatggcagtctagcagaggaagaggtagtaattagatctgaaaatttcatgagaaatgataaaatcataatagtacagctaaatgaatctatagaaattaattgtacaagacccaactataatacaagaaaaggtttccatataggaccagggagagcaatttatacaggacaaataataggagatatcagacaagcacattgtaacattagtggaataaaatggaagaaggctttaaaacagatagttggaaaattaagagaacaatttgggaataaaacaatagtatttaatcagtcctcaggaggggacctagaaattgaaacacacagttttaattgtggaggggaatttttctactgcaatacaacacaactgtttaataatacttggccgtcaaataatactgacggagatattaacgaaaacatcacactcacactcccatgcagaataaaacaaattataaacatgtggcagaaagtaggaaaagccatgtatgcccctcccatcaaaggacaaattagatgttcatcaaatattacagggctgctactaatgagagatggtggtaacgacaccaccaaggcaaacgacaccgaggtcttcagacctggaggaggggagatgagggacaattggagaagtgaattatataaatataaagtagtaaaaattgaaccattaggaatagcacccaccaaggcaaagagaagagtggtgcagagagaaaaaagaggagtaggattaggagctatgttccttgggttcttgggagcagcaggaagcact

SEQ ID NO: 92 - protein, 467 aa, Human immunodeficiency virus type 1
SAEEKWVTVYYGVPVWKEAKTTLFCASDAKAYDTEAHNVWATHACVPTDPNPQEVVLENVTENFNMWKNDMVEQMHEDVISLWDQSLKPCVELTPLCVTLNCTNLNCTNNTSSEIKNCSFYVTTSMEGKVKKHATFYSLDIVRTTESNISYRLISCNTSVITQACPKISFEPIPIHYCAPAGFAILKCNNKTFNGTGPCTNVSTVQCTHGIKPVVSTQLLLNGSLAEEEVVIRSENFMRNDKIIIVQLNESIEINCTRPNYNTRKGFHIGPGRAIYTGQIIGDIRQAHCNISGIKWKKALKQIVGKLREQFGNKTIVFNQSSGGDLEIETHSFNCGGEFFYCNTTQLFNNTWPSNNTDGDINENITLTLPCRIKQIINMWQKVGKAMYAPPIKGQIRCSSNITGLLLMRDGGNDTTKANDTEVFRPGGGEMRDNWRSELYKYKVVKIEPLGIAPTKAKRRVVQREKR

SEQ ID NO: 93 - DNA, 1691 bp, Human immunodeficiency virus type 1
agaaagagcagaagacagtggcaatgagagtgaaggagatcaagaggagttatcagctcttgttgaaagggggcatcttgctccttgggatattgatgatctgtagtgctacagacaacttgtgggtcacagtatattatggggtacctgtatggaaagaagcaaccaccactctattttgtgcatcagatgctaaagcccataatacagaggtacacaatgtttgggccacacatgcctgtgtacccacagaccctgacccacaagaagtagtattggaaaatgtgacagaaaattttaacatgtggaaaaatgacatggtagaacagatgcatgaggatataatcagtttatgggatcaaagcctaaagccatgtgtaaaattaaccccactttgtgttactttaaattgcactaatgctaaaatgaacaatactgctgataccaatgctactaatactgttaatatcagcaaggaagaaatggaagaaataaaaaactgctctttcaatgtcaccacaagcttaagagataagatgcagagccaatatgcattgttttataaacttgatatagtaccaatagataatagtagtagtatagataatagtagtaatacatgtaatagtaatagtacacataataatagtagtacatgtaataattatgctaattatagattgataagttgtgacacctcagtcattacacaggcctgtccaaaggtatcctttgagccaattcccatacattattgtgccccggctggttttgcgattctaaagtgtaataataagacgttcaatggatcaggaccatgtaaaaatgtcagcacagtacaatgtacacatggaatcaggccagtagtatcaactcaactgctgttaaatggcagtctagcagaagaaggggtagttattagatctgagaatttcacaaacaatgctaaaaccataatagtacagatacatgaacctatagaaattaattgtacaagacccaacaacaatacaagaaaaagtataactataggaccaggaagagcgttttatgcaacaggagacataataggagatataagacaagcacattgtaaccttagtaaagcaagatggaatgatactttaaaacagatagttacaaaattaagagaacagtttagaaataaaacaataaattttactcaatcctcaggaggggacccagaaattgtgatgcacagttttaattgtggaggggaatttttctactgtaatacaacacaactgtttaatagtacttggttgtctaatagtacttggaatgatactgaaaggtcaaatggcactgaaactattacactcccatgcagaataaagcaagttataaacaggtggcaggaagtaggaaaagcaatgtatgcccctcccatcagtggaataattagatgttcatcaaatattacagggctgctattaacaagagatggtggtaatagtaatgacactccgactgatactgagatcttcagacctggaggaggagatatgagggacaattggagaagcgaattatataaatataaagtagtaaaaattgaaccattaggaatagcacccaccaaggcaaagagaagagtggtgcagagagaaaaaagagcagcgggaataggagctctgttccttgggttcttgggagcagcaggaagcact

SEQ ID NO: 94 - protein, 511 aa, Human immunodeficiency virus type 1
SATDNLWVTVYYGVPVWKEATTTLFCASDAKAHNTEVHNVWATHACVPTDPDPQEVVLENVTENFNMWKNDMVEQMHEDIISLWDQSLKPCVKLTPLCVTLNCTNAKMNNTADTNATNTVNISKEEMEEIKNCSFNVTTSLRDKMQSQYALFYKLDIVPIDNSSSIDNSSNTCNSNSTHNNSSTCNNYANYRLISCDTSVITQACPKVSFEPIPIHYCAPAGFAILKCNNKTFNGSGPCKNVSTVQCTHGIRPVVSTQLLLNGSLAEEGVVIRSENFTNNAKTIIVQIHEPIEINCTRPNNNTRKSITIGPGRAFYATGDIIGDIRQAHCNLSKARWNDTLKQIVTKLREQFRNKTINFTQSSGGDPEIVMHSFNCGGEFFYCNTTQLFNSTWLSNSTWNDTERSNGTETITLPCRIKQVINRWQEVGKAMYAPPISGIIRCSSNITGLLLTRDGGNSNDTPTDTEIFRPGGGDMRDNWRSELYKYKVVKIEPLGIAPTKAKRRVVQREKR

SEQ ID NO: 95 - DNA, 1616 bp, Human immunodeficiency virus type 1
agaaagagcagaagacagtggcaatgagagtgaaggggatcaggaagaattgtcagcgctggtggagatggggcatcatgctccttgggatgttgatgatctgtaaagctgcagaccaattgtgggtcacagtctattatggggtacctgtgtggaaggaagcaaccaccactttattttgtgcatcagatgctaaagcatataagacagaggtacataatgtttgggccacacatgcctgtgtacccacagaccccaatccacaagaaatagacttggcaaatgtgacagaaaattttaacatgtggaaaaataacatggtagaacagatgcatgaggatataatcagtttatgggatcaaagcctaaagccatgtgtaaaactaaccccactctgtgttactttaaattgtactgatgacttagaaaattgtaaaaataccactaataataatgccgctaataataataatacctgcgacatgccaggagaaataaagaactgctctttcaatatcaccgcaggtataagagataagatgcagaaagaatatgcactttttaatacacttgatgtagtaccaataggtgatgagaatgataataccagttataggttaataagttgtaatacctcagtcattacacaggcctgtccaaaggtatcctttgagccaattcccatacattattgtgccccggctggttttgcgattctaaagtgtaaagataagaaattcaatgggacaggacagtgtaaaaatgtcagcacagtacaatgtacacatggaattaagccagtagtatcaactcaattactgttaaatggcagtctagcagaagaagaggtagtaattaggtctgaaaatttcacaaacaatgctaaaaccataatagtacagctgaaagaacctgtacaaattaattgtacaaggcccaacaacaatacaagaaaaagcatacatataggaccagggagagcattttatgcaacaggacaaataataggagatataagacaagcacattgtaacattagtagtataaaatggaataacactttaaaacagatagttaaaaaattaagagaacaatttaataaaacaataatctttaatcaatcctcaggaggggacccagaaattgtaatgcacatttttaattgtagaggggaatttttctactgtaatacaacacaactgtttaatagcacttggaatattactgaagggtcaaatgacaatattacaggcaggtcaaatgacactatcacgctcccatgcaagataagacaaattgtaaacatgtggcaggaagtaggaaaagcaatgtatgctcctcccatcagaggacaaattaactgtacatcaaatattacagggctgctgttaacaagagatggtggtaaaaatatcagcgagtccgaaaccttcagacctggaggaggaaatatgaaggacaattggagaagtgaattatataaatacaaagtagtacaaattgaaccattaggagtagcacccaccaaggcaaagagaagagtggtgcagagagaaaaaagagcagtgggaataggagctctgttccttgggttcttgggagcagcaggaagcact

SEQ ID NO: 96 - protein, 486 aa, Human immunodeficiency virus type 1
KAADQLWVTVYYGVPVWKEATTTLFCASDAKAYKTEVHNVWATHACVPTDPNPQEIDLANVTENFNMWKNNMVEQMHEDIISLWDQSLKPCVKLTPLCVTLNCTDDLENCKNTTNNNAANNNNTCDMPGEIKNCSFNITAGIRDKMQKEYALFNTLDVVPIGDENDNTSYRLISCNTSVITQACPKVSFEPIPIHYCAPAGFAILKCKDKKFNGTGQCKNVSTVQCTHGIKPVVSTQLLLNGSLAEEEVVIRSENFTNNAKTIIVQLKEPVQINCTRPNNNTRKSIHIGPGRAFYATGQIIGDIRQAHCNISSIKWNNTLKQIVKKLREQFNKTIIFNQSSGGDPEIVMHIFNCRGEFFYCNTTQLFNSTWNITEGSNDNITGRSNDTITLPCKIRQIVNMWQEVGKAMYAPPIRGQINCTSNITGLLLTRDGGKNISESETFRPGGGNMKDNWRSELYKYKVVQIEPLGVAPTKAKRRVVQREKR

SEQ ID NO: 97 - DNA, 1640 bp, Human immunodeficiency virus type 1
agaaagagcagaagacagtggcaatgagagtgaaggggatcaggaagaattatcagcactggtggagatgggggaccatgctcctttggttattgatgatctgtagtgctgcagaacaattgtgggtcacagtttactatggggtacctgtgtggaaagaagcaaccaccactctattttgtgcatcagatgccaaagcatatgatccagaggcacataatgtttgggccacacatgcctgtgtacccacagaccccaacccacaagaattagtattggcaaatgtgacagaaaattttaacatgtggaaaaataacatggtagaacagatgcaggaggatataatcagtttatgggatcaaagcctaaagccatgtacaaaattaaccccactctgtgttactttaaattgtactgatgtggagtataataatgaaactgcttccaataatactgcttccaatactactattattaatgggggaacaatagagggagatggaataaaaaactgctctttcaatatcaccaccagcctaaataaggtgcagaaagaaaatgcatatttttataatcttgatgtagtacaaatagataatagtgataataccagctataggttagtaagttgtaacacctcagtcattacacaggcctgtccaaagatatcttttgagccaattcccatacattactgtaccccggctggttttgcgattctaaagtgtaatgataaaaagttcaatggaacaggaccatgtaaaaatgtcagcacagtacaatgtacacatggaattaagccagtagtatcaactcaactgttgttaaatggcagtctagcagaagaagaggtagtaattagatcagaaaatttcacagataatgcaaaaatcataatagtacagctgaatgaatctatggaaattaattgtgcaagacccaacaacaatacaagaaaaggtatacatatgggaccagggaaagcattttatgcaacaggagccataataggagatatacgaagagcacattgcaacattagtcaaacaaagtggaacaatgccctaaaacagatagctataaagttaagagaacaatttgggaataaaacaatagtctttacaaactcctcaggaggggacccagaaattgtaatgcacagttttaactgtggaggggagtttttctactgtaatacatcactcctgtttaatagtatctggaatagtactactttgtcaaatagcactacaggagatggaaatatcacactcccatgcagaataaaacaaattataaatatgtggcagaaagtagggaaagcaatgtatgcccctcccatccaaggactaattaaatgtacatcaaatatcacagggatgttattaataagagatggtggtaacatcaactgcactgagactaataccaccaactgcaatgagactgagaccttcagacctgtaggaggagatatgagggacaattggagaagtgaattatataaatataaagtagtaaaaattgaaccattaggaatagcacccactaaggcaaagagaagagtggtgcagagagaaaaaagagcagcgggactaggagctttgttccttgggttcttgggagcagcaggaagcact

SEQ ID NO: 98 - protein, 494 aa, Human immunodeficiency virus type 1
SAAEQLWVTVYYGVPVWKEATTTLFCASDAKAYDPEAHNVWATHACVPTDPNPQELVLANVTENFNMWKNNMVEQMQEDIISLWDQSLKPCTKLTPLCVTLNCTDVEYNNETASNNTASNTTIINGGTIEGDGIKNCSFNITTSLNKVQKENAYFYNLDVVQIDNSDNTSYRLVSCNTSVITQACPKISFEPIPIHYCTPAGFAILKCNDKKFNGTGPCKNVSTVQCTHGIKPVVSTQLLLNGSLAEEEVVIRSENFTDNAKIIIVQLNESMEINCARPNNNTRKGIHMGPGKAFYATGAIIGDIRRAHCNISQTKWNNALKQIAIKLREQFGNKTIVFTNSSGGDPEIVMHSFNCGGEFFYCNTSLLFNSIWNSTTLSNSTTGDGNITLPCRIKQIINMWQKVGKAMYAPPIQGLIKCTSNITGMLLIRDGGNINCTETNTTNCNETETFRPVGGDMRDNWRSELYKYKVVKIEPLGIAPTKAKRRVVQREKR

SEQ ID NO: 99 - DNA, 1649 bp, Human immunodeficiency virus type 1
agaaagagcagaagacagtggcaatgagagtgaaggagatcaggaggaattgtcagcactggtggtggaaatggggcaccatgctccttgggatgttgatgatctgtagtgctaacgagcaattgtgggtcacagtctattatggggtacctgtgtggaaagaagcaaccaccactctattttgtgcatcagatgctaaagcatatgacacagaggcacataatgtttgggccacacatgcctgtgtacccacagaccctaacccacaagaagtagtattggtaaatgtgacagaaaattttaatatgtggaaaaataacatggtagaacagacgcaggaggatataatcagtttatgggatcaaagcctaaagccatgtgtaaaattaaccccactctgtgttactctaaattgcactgattggaccctacattgcaataataatgatactaattgcactactttaaaaaatgatactaaaaccaataataatagtagtttgagaacaatggagggaggagaagtaaaaaactgctctttcaatgtcaccacaagcctaagagataaggagcgaaaagaatatgcacttttttataaacttgatgtagtaccaataggtaatgataatacaagctatacgctgataaattgtaacacctcaaccattacacaggcctgtccaaaggtaacctttgaaccaattcccatacattattgtaccccggctggttttgcgcttctaaagtgtaatgataagaagttcaatggaacaggacagtgtaaaaatgtcagcacagtacaatgtacacatggaattaggccagtagtatcaactcaattgctgctaaatggcagtctatcagaaggagaggtaatgattagatctgaaaatttcacaaacaatgctaaaaccataatagtacagctgaatgaatctatagcaattaattgtacaagacccaacaacaatacaagaaaaagtataactataggaccggggagagcattttttacaacaggagaaataacaggagatataagacaagcacattgtaaccttagtgcagtacaatggaataacacattaaaacagatagttgcaaaattaagggaacaatttgggaataatacaataagctttaataaatccgcaggaggggacccagaaattgtaatgcacagttttaattgtggaggggaatttttctactgtgatacaacacagctgtttaatagtacttgggataatgacacagacttaagtattaagaatgagactacagaatcaaacaacaaaactatcacactcccgtgcagaataaaacaaattataaacagatggcaggaagtaggaaaagcaatgtatgcccctcccatcagaggacaaattaaatgttcatcaaatattacagggctactattaacaagagatggtggtatgaacaatagcgccaacgagaccttcagacctggaggaggagatatgagggacaattggagaagtgaattatataaatataaagtagtaaaaattgagccattaggggtagcacccaccaaggcaaagagaagagcggtgcaaagagaaaaaagagcagtgggaataggagctgtgttccttgggttcttgggagcagcaggaagcact

SEQ ID NO: 100 - protein, 496 aa, Human immunodeficiency virus type 1
SANEQLWVTVYYGVPVWKEATTTLFCASDAKAYDTEAHNVWATHACVPTDPNPQEVVLVNVTENFNMWKNNMVEQTQEDIISLWDQSLKPCVKLTPLCVTLNCTDWTLHCNNNDTNCTTLKNDTKTNNNSSLRTMEGGEVKNCSFNVTTSLRDKERKEYALFYKLDVVPIGNDNTSYTLINCNTSTITQACPKVTFEPIPIHYCTPAGFALLKCNDKKFNGTGQCKNVSTVQCTHGIRPVVSTQLLLNGSLSEGEVMIRSENFTNNAKTIIVQLNESIAINCTRPNNNTRKSITIGPGRAFFTTGEITGDIRQAHCNLSAVQWNNTLKQIVAKLREQFGNNTISFNKSAGGDPEIVMHSFNCGGEFFYCDTTQLFNSTWDNDTDLSIKNETTESNNKTITLPCRIKQIINRWQEVGKAMYAPPIRGQIKCSSNITGLLLTRDGGMNNSANETFRPGGGDMRDNWRSELYKYKVVKIEPLGVAPTKAKRRAVQREKR

SEQ ID NO: 101 - DNA, 1610 bp, Human immunodeficiency virus type 1
agaaagagcagaagacagtggcaatgagagtgaaggggatcaggaagaattgcttacggaaatggggcaccatgctccttgggatattaatgatctgtagtgctgcaggaaatttgtgggtcacagtctattatggggtgcctgtgtggaaagaagcaaccaccactctattctgtgcatcagatgctaaagcatatgctacagaggcacataatgtttgggccacacatgcctgtgtacccacagaccctaacccacaagaagtagtaatggaaaatgtgacagaaaattttaacatgtggaaaaataacatggtagaacagatgcatgaggatataatcagtttatgggatcaaagcctaaagccatgtgtaaaattaaccccactctgtgttactctaaattgcactgactgtgtgacttcaaattgcactaagttgaagaatgtcactaataatgctaatattagcaagatggagatggaggagggagaaatgaaaaactgctcttttaacatcacctcaggcatgagagataagatgaagaaagaatatgcatttttttataaacttgatatagtaccaataagtaatgataatactagctatagattgataagttgtaatacctcagtcattacacaggcatgtccaaaagtatcctttgagccaattcccatacattattgtgccccggctggttttgcgattctaaaatgtaatgataagaagttcaatggaacaggaccatgtaaaaatgtcagcacagtacaatgtacacatggaattaggccagtagtgtcaactcaattgctgttaaatggcagcctagcagaagaagaggtagtaattagatctgaaaatttaacggacaatggtaaaaccataatagtacagctgaacgaatctgtacacattaattgtacaagacccagcaacaataccagaaaaagtatacatataggaccagggaaagcattttatgcaacaggacaaataataggagatataagacaagcacactgtaacattagtgaaaaacaatggaataaaactttaagccagatagttaaaaaattaagagaacaatttaaaaataaaacaatagtctttaatcaatcctcaggaggggacccagaaattgtaatgcacagttttaattgtggaggagaatttttctactgtaattcaacacaactatttaatactacttggaattggaatgatactgaagggtcaaataacactgaaagaaatgaaagaaatattacactcccatgcagaataaaacaaattgtaaacaggtggcaggaagtaggaaaagcaatgtatgcccctcccatcagcggaccaattagctgttcatcaaatattacagggctgctattaacaagagatggtggtctcccaaacaatactgagaccttcagaccagaaggaggaaatatgaaggacaattggagaagtgaattatataaatataaagtagtaaaaattgaaccattaggaatagcacccaccaaggcaaagagaagagtggtgcaaagagaaaaaagagcagtgacattaggagctatgttccttgggttcttgggagcagcaggaagcact

SEQ ID NO: 102 - protein, 486 aa, Human immunodeficiency virus type 1
SAAGNLWVTVYYGVPVWKEATTTLFCASDAKAYATEAHNVWATHACVPTDPNPQEVVMENVTENFNMWKNNMVEQMHEDIISLWDQSLKPCVKLTPLCVTLNCTDCVTSNCTKLKNVTNNANISKMEMEEGEMKNCSFNITSGMRDKMKKEYAFFYKLDIVPISNDNTSYRLISCNTSVITQACPKVSFEPIPIHYCAPAGFAILKCNDKKFNGTGPCKNVSTVQCTHGIRPVVSTQLLLNGSLAEEEVVIRSENLTDNGKTIIVQLNESVHINCTRPSNNTRKSIHIGPGKAFYATGQIIGDIRQAHCNISEKQWNKTLSQIVKKLREQFKNKTIVFNQSSGGDPEIVMHSFNCGGEFFYCNSTQLFNTTWNWNDTEGSNNTERNERNITLPCRIKQIVNRWQEVGKAMYAPPISGPISCSSNITGLLLTRDGGLPNNTETFRPEGGNMKDNWRSELYKYKVVKIEPLGIAPTKAKRRVVQREKR

SEQ ID NO: 103 - DNA, 1610 bp, Human immunodeficiency virus type 1
agaaagagcagaagacagtggcaatgagagtgaaggggatcaggaagaattatcagcactggtggaaatggggcacgatgctccttggaatgttgatgatctgtagtgctgcagaacaattgtgggttacagtctattatggggtacctgtgtggaaggaagcaaagaccactctattctgtgcatcagatgctaaagcatatgaaacagaggtacataatgtttgggccgcacatgcctgtgtacccacagaccccaacccacaagaagtagtattggcaaatgtgacagaaaattttaatatgtgggacaacaacaaggtagaacagatgcaggaggatataatcagtttatgggatgaaggcctaaagccatgtgtaaaattaaccccactctgtattactctaaattgcactgacccctgtgatgctcaaaatagcactcgaagttgcacttatttgaatgatacggtggaagaggagagaggacaaataagaaattgctctttcaatatctccacaaagctagaaaatagaaggcagacaggatatgcagtttttgataaacttgatttagtaccagtagatggtggtaataatactgtcagatataggttgataaattgtaacacctcagtcattacacaagcatgtccaaaggtatcctttgaaccaattcccatacattattgtaccccagctggttttgcgattctaaagtgtaatgataagaagttcaatggaacagggccatgcacaaatgtcagcacagtacaatgcacacatggaattaggccagtagtgtcaactcaactgctgttaaatggcagtctggcagaagaagaggtagtaattagatctaaaaatttcacagacaatactaaaactataatagtacagctgaatgaatctgtacaaattaattgtacaagacccaacaacaatacaagacagagtacacctatgggaccagggaaagcactttacacaacacagataataggagacataagacaagcacattgtaacattagtacaaaagattggaatgacaccttaaaaaagatagttacaaaattagaagaacaatttgaaaataaaacaataagctttaatcaatcctcaggaggggacccagaaattgtaaagcacagttttaattgtggaggggaattcttctactgtgatacaacaaaactatttaatagtacttggcctgcaagtatcaattataccactggagtaaatatcactggagttatcacactcccatgtagaataaaacaaattataaacaggtggcagggaggaggaaaggcaatgtatgctcctcccatcagtggaccaattagatgttcatcaaatattacagggctgctattaacaagagatggtggtgatgacaataatggggctgagacctttagacctggaggaggagatatgagggacaattggagaagtgaattatataaatataaagtagtaaaaattgaaccattaggagtagcacccaccaaggcaaagagaagagtggtgcagagagaaaaaagagcagtgggaacactaggagctatgttccttgggttcttgggagcagcaggaagcact

SEQ ID NO: 104 - protein, 483 aa, Human immunodeficiency virus type 1
SAAEQLWVTVYYGVPVWKEAKTTLFCASDAKAYETEVHNVWAAHACVPTDPNPQEVVLANVTENFNMWDNNKVEQMQEDIISLWDEGLKPCVKLTPLCITLNCTDPCDAQNSTRSCTYLNDTVEEERGQIRNCSFNISTKLENRRQTGYAVFDKLDLVPVDGGNNTVRYRLINCNTSVITQACPKVSFEPIPIHYCTPAGFAILKCNDKKFNGTGPCTNVSTVQCTHGIRPVVSTQLLLNGSLAEEEVVIRSKNFTDNTKTIIVQLNESVQINCTRPNNNTRQSTPMGPGKALYTTQIIGDIRQAHCNISTKDWNDTLKKIVTKLEEQFENKTISFNQSSGGDPEIVKHSFNCGGEFFYCDTTKLFNSTWPASINYTTGVNITGVITLPCRIKQIINRWQGGGKAMYAPPISGPIRCSSNITGLLLTRDGGDDNNGAETFRPGGGDMRDNWRSELYKYKVVKIEPLGVAPTKAKRRVVQREKR

SEQ ID NO: 105 - DNA, 1589 bp, Human immunodeficiency virus type 1
agaaagagcagaagacagtggcaatgaaagtgagggagaccaggaggaattattgggacttgtggagatggggcacgatgctccttgggatgttaatgatctgtagtgctacagaacaattatgggtcacaatctattatggggtacctgtgtggaaagaagcagataccactctattttgtgcatcagatgctaaagcatatgatacagagaaacataatgtttgggccacacatgcctgtgtacccacagaccctaacccacaagaagtagtaatggcaaatgtgacagaaaattttaacatgtggaaaaataacatggtagaacagatgcagaaggatattatcagtctatgggatgaaagcttaaaaccatgtgtaaaattaaccccactctgtatcactttaaattgcaatactttaaattgcacacataacaataattgtagtagtttgagagaagaaatgacaaactgctccttcaatgccacctcaaaattgagagataaggttcggaaacaatatgcaattttttctaaacttgatgtggtaaaactagatagtaataataatgaaacagaatataggttgataaattgtaacacctcagtcgttacacaggcctgtccaaaggtatcatttgagccaattcccatacattattgtgccccggctggttttgcgattctaaagtgtaacaataagacattcgatggaaaaggaccatgtacaaatgtcagcacagtacaatgtacacatggaattaggccagtagtgtcaactcaactgctgttaaatggcagtctagcagaagaagggatagtaattagatctgacaatatctcagacaatactaaaaccataatagtacagctgaatgaatctgtagcaattaattgttcaagaccccacaacaatacaagaaaaagtatacatatagcaccagggagagccttttatgcaacaggagacataataggagatataagacaagcacattgtaacattagtagaaaacaatggaataaaactttagaacaggtagctaaaaaattaagagaaaaatttgtaaataaagcaattatctttaaaaactcctcaggaggggacccagaaattgtaatgcacagttttaattgtagaggggaatttttctactgtaatacaacaccactgtttaatgggacttggaatggtactaatttgagtactaataaggagtcaaatgacacaatcatactccaatgtagaataaaacaaattataaacatgtggcaggaagtaggaaaagcaatgtatgcccctcccattgcaggacaaattaactgttcatcaaaaattacagggctactattaacaagagatggtagtagcacaaatgggacaaatgagactgagatcttcagacctggaggaggagatatgagggacaattggagaagcgaattatataaatataaagtagtaagagttgaaccaataggaatagcacccaccaaggcaaagagaagagtggtgcagagagaaaaaagagcagtgggaacgttaggagctatgttccttgggttcttgggagcagcaggaagcact

SEQ ID NO: 106 - protein, 476 aa, Human immunodeficiency virus type 1
SATEQLWVTIYYGVPVWKEADTTLFCASDAKAYDTEKHNVWATHACVPTDPNPQEVVMANVTENFNMWKNNMVEQMQKDIISLWDESLKPCVKLTPLCITLNCNTLNCTHNNNCSSLREEMTNCSFNATSKLRDKVRKQYAIFSKLDVVKLDSNNNETEYRLINCNTSVVTQACPKVSFEPIPIHYCAPAGFAILKCNNKTFDGKGPCTNVSTVQCTHGIRPVVSTQLLLNGSLAEEGIVIRSDNISDNTKTIIVQLNESVAINCSRPHNNTRKSIHIAPGRAFYATGDIIGDIRQAHCNISRKQWNKTLEQVAKKLREKFVNKAIIFKNSSGGDPEIVMHSFNCRGEFFYCNTTPLFNGTWNGTNLSTNKESNDTIILQCRIKQIINMWQEVGKAMYAPPIAGQINCSSKITGLLLTRDGSSTNGTNETEIFRPGGGDMRDNWRSELYKYKVVRVEPIGIAPTKAKRRVVQREKR

SEQ ID NO: 107 - DNA, 1689 bp, Human immunodeficiency virus type 1
agaaagagcagaagacagtggcaatgagagtgacggggatcaggaagaattacttatggagatggggcaccttattcctggggatattgatgatctgtaaggctgcagaaaacttgtgggtcacagtctattatggggtacctgtgtggaaagacgcaaccaccactctattttgtgcatcagatgctaaagctgttgatacagaagtacacaatgtgtgggccacacatgcctgtgtacccacagacccaaacccacaagaagtagtattgtataatgtgacagaaaattttaacatgtggaaaaataacatggtagaacagatgcatgaggatataatcagtttatgggatgaaagtctaaagccatgtgtaaagctaaccccactctgtgttactttgaattgcactgatgaattgaatatcaatactaataatacctgtagtaataccagtagtggcactaataataccagtaattgcactaatgctgagtcgactatcatcagtaccagtaatagtagcaataccagaaatatcagtgatagtagcaagatagagaaaggagaaataaaaaactgtactttcaatatcaccacaagcataagagataaggtgcagaaagaatatgcactgttttataaacttgatgtagtaccaacaggtagtgacaatactagctataggttgataagttgtaatacctcagtcattacacaggcctgtccaaaggtaacctttgagccaattcctatacattattgtgccccggctggttatgcgattgtaaagtgcaacaataagacattcagtggaaaaggaccatgtaaaaatgtcagcacagtacaatgcacacatgggattaagccagtagtatcaactcagttgctgttaaatggcagtctggcagaaaaagatatagtaattagatctgacaacttctcaaacaatgctaaaaccataatagtgcagctgaacacatctgtagaaattaattgtacaagacccagcaacaatacaagaaaaagtattcatataggaccagggagagcattttatgcaacagatataataggagatataagacaagcacattgtaacattagtgaagaaaattggtataaaactctaaagcaggtagctatgaaattaggagaacagtttcagaataaaaaaatagtctttaatcaatcctcaggaggggacccagaaattgtaatgcacagttttaattgtggaggggaatttttcttctgtagtacatcacaactgtttaatagtacttggaattatagtacttgggatgaaatagaaaataaaactcaaggaaatggcacactcacactcccatgcagaataaaacaaattgtaaacatgtggcaggaagtaggaaaagcaatgtatgcccctcctatcaaaggaaatattacatgttcatcaaagattacagggttgctattaacaagagatggtggtaagactaatatcaatggcaccgagatcttcagaccaggaggaggagatatgagggacaattggagaagtgaactatataaatataaagtagtaaaaattgaaccattaggaatagcacccaccaaggcaaagagaagagtggtgcagagagaaaaaagagcagtgggaataggagctatgttccttgggttcttgggaggcagcaggaagcact

SEQ ID NO: 108 - protein, 512 aa, Human immunodeficiency virus type 1
KAAENLWVTVYYGVPVWKDATTTLFCASDAKAVDTEVHNVWATHACVPTDPNPQEVVLYNVTENFNMWKNNMVEQMHEDIISLWDESLKPCVKLTPLCVTLNCTDELNINTNNTCSNTSSGTNNTSNCTNAESTIISTSNSSNTRNISDSSKIEKGEIKNCTFNITTSIRDKVQKEYALFYKLDVVPTGSDNTSYRLISCNTSVITQACPKVTFEPIPIHYCAPAGYAIVKCNNKTFSGKGPCKNVSTVQCTHGIKPVVSTQLLLNGSLAEKDIVIRSDNFSNNAKTIIVQLNTSVEINCTRPSNNTRKSIHIGPGRAFYATDIIGDIRQAHCNISEENWYKTLKQVAMKLGEQFQNKKIVFNQSSGGDPEIVMHSFNCGGEFFFCSTSQLFNSTWNYSTWDEIENKTQGNGTLTLPCRIKQIVNMWQEVGKAMYAPPIKGNITCSSKITGLLLTRDGGKTNINGTEIFRPGGGDMRDNWRSELYKYKVVKIEPLGIAPTKAKRRVVQREKR

SEQ ID NO: 109 - DNA, 1616 bp, Human immunodeficiency virus type 1
agaaagagcagaagacagtggcaatgagagtgatggggatcaggaggaattatcagcacttgtggagatggggcaccatgctccttgggatgttgatgatctgtaaaatcactacaggaaaaacgtgggtcacagtctattatggggtacctgtgtggaaagaagcaactaccactctattttgtgcatcagatgctaaagcatatgatacagaggtacataatgtttgggccacacatgcctgtgtacccacagaccccaacccacaaaaaatagtaatggtaaatgtgacagaggaatttaacatgtggaaaaataacatggtagaacagatgcatgaggatataatcagtttatgggatcagagcctaaagccatgtgtaaaattaaccccactctgcgttactttaaattgcaatgatactacggagagaaattgcactggtcccgatggtagaaaaataaattgcactgaagtgaaaaattgctctctcaatatcaccacaaacataagagataaggtgcagaaagaatatgcacttttttatagaactgatgtggtgccaatagataataataatactagtaccagcagtcatagctataggttgataagttgtaatacctcagtccttacacagacctgtccaaaagtatcctttcagccaattcccatacattattgtgccccggctggttttgcgattctaaaatgtaacaataggacattcaatggaaaaggagaatgtggtaatgtcagcacagtacaatgtacacatggaattaagccagtagtatcaactcagctgctgctaagtggcagtctagcagaacaagagatagtgcttagatctgacaacttctcagacaatgctaaaaccataatagtacagctgactaaacctgtagaaattaattgtacacgacccaacaacaatacaagaaaaagtatagctataggaccagggagagcatttattgcaacaggagacataataggagatataagacaggcacattgtaacattagtaaagtagcatggaataacactatagaagaggtagcaagaaaattaagcaaacaatttgggaatagatcaataacctttaatcaatcctcaggaggggacccagaaattgtaatgcacagttttaattgtggaggggaatttttctactgtaatacaacaggactatttagtagtacttggaatgttactgataattggaatgctactgaagaggcaaataccactagggtaaatatcacactcccatgcagaataagacaaattgtaaacatgtggcaggaagtaggaaaagcaatgtatgcccctcccatcagaggacaaattagttgctcatcaaatattacaggactgatattaacaagagatggtggtaaccagagcaacgagacgacccctgagatctttagacctggaggaggagatatgagggacaattggagaagtgaattatataaatataaagtagtaaaaattgaacctataggaatagcacccaccaaggcaaagagaagagtggtgcagagagaaaaaagaggggtactaggagctatgttccttgggttcttgggagcagcaggaagcact

SEQ ID NO: 110 - protein, 487 aa, Human immunodeficiency virus type 1
KITTGKTWVTVYYGVPVWKEATTTLFCASDAKAYDTEVHNVWATHACVPTDPNPQKIVMVNVTEEFNMWKNNMVEQMHEDIISLWDQSLKPCVKLTPLCVTLNCNDTTERNCTGPDGRKINCTEVKNCSLNITTNIRDKVQKEYALFYRTDVVPIDNNNTSTSSHSYRLISCNTSVLTQTCPKVSFQPIPIHYCAPAGFAILKCNNRTFNGKGECGNVSTVQCTHGIKPVVSTQLLLSGSLAEQEIVLRSDNFSDNAKTIIVQLTKPVEINCTRPNNNTRKSIAIGPGRAFIATGDIIGDIRQAHCNISKVAWNNTIEEVARKLSKQFGNRSITFNQSSGGDPEIVMHSFNCGGEFFYCNTTGLFSSTWNVTDNWNATEEANTTRVNITLPCRIRQIVNMWQEVGKAMYAPPIRGQISCSSNITGLILTRDGGNQSNETTPEIFRPGGGDMRDNWRSELYKYKVVKIEPIGIAPTKAKRRVVQREKR

SEQ ID NO: 111 - DNA, 1694 bp, Human immunodeficiency virus type 1
agaaagagcagaagacagtggcaatgagagtgaaggggatcaggaagaattgtcactcctggagatggggcaccatgctcctatggagatggggcaccatgctccttgggttgttaatgatgatctgtaatgctaaagaagaaaaaacgtgggtcacagtatattatggggtacctgtgtggaaagaagcgaccaccactctattttgtgcatcagatgctaaagcatatgacacagaggtacataatgtttgggccacacatgcctgtgtacctacagaccccgacccacaggaaatattcttggaaaatgtgacagaaaattttaacatgtggaaaaataacatggtagaacagatgcatgaggatataatcagtttgtgggatcaaagcctaaagccatgtgtaaaattaaccccactctgtgttacgttaaattgcactaatgtgaggattactagcataaattgcactgatgcgagtagtaatagcactggtgtggaaaaaactactagcactaatggggggaatgttactatttgtaatactactgaggagataaaaaactgctccttcaacatcaccaccaacatgaaagataagatacagaagacatatgcactgttttataaacttgatgtagaaccaatagataaggggaatgagaatagcaccagctatagggtgataagttgtaacacttcaaccattacacaggcctgtccaaaggtatcctttgagccaattcctatacattattgtgccccggctggttttgcgattctaaaatgtagagataaaaggtttaatggaacaggaccatgtacaaatgtcagcacagtacaatgtacacatggaattaaaccagtagtatcaactcaactgctgttaaatggcagtctagcagaagaagagatagtacttagatctgaaaatttctcgaacaatgctaaaaacatattaatacagctaaaagaacctgtagaaattaattgtacaagacccaacaacaatacaagaaaaggtatacatataggaccaggaagagcattttatggaacagatataataggagatataagacaagcacattgtaacattagcagagaaaaatggaatagcactttaagtcagatagttgataaattaagagaacaatatgggaaaaataaaacaataatctttgatcaaccatcaggaggggacccagaaattgtaacgcacagttttaattgtggaggagaatttttctactgtaattcaacacaactgtttaatagcacttggtatgataatagtacttggaatgagaataaaaatcacactaaaaatgacacaatcacgctcccatgcagaataaagcaatttataaacatgtggcaggaagtaggaaaagcaatgtatgcccctcccatcagaggacaaattaaatgctcatcaaatattacagggctaataataacaagagatggggggaacaacaacagcgagaccttcaacaacgagaccttcagacctggaggaggaaatatgaaggacaattggagaagtgaattatataaatataaagtagtaagaattgagccattaggagtagcacccaccagggcaaagaggagagtggtgcagagagaaaaaagagcagtgggaataggagctgtgttccttgggttcttgggagcagcaggaagcact

SEQ ID NO: 112 - protein, 504 aa, Human immunodeficiency virus type 1
NAKEEKTWVTVYYGVPVWKEATTTLFCASDAKAYDTEVHNVWATHACVPTDPDPQEIFLENVTENFNMWKNNMVEQMHEDIISLWDQSLKPCVKLTPLCVTLNCTNVRITSINCTDASSNSTGVEKTTSTNGGNVTICNTTEEIKNCSFNITTNMKDKIQKTYALFYKLDVEPIDKGNENSTSYRVISCNTSTITQACPKVSFEPIPIHYCAPAGFAILKCRDKRFNGTGPCTNVSTVQCTHGIKPVVSTQLLLNGSLAEEEIVLRSENFSNNAKNILIQLKEPVEINCTRPNNNTRKGIHIGPGRAFYGTDIIGDIRQAHCNISREKWNSTLSQIVDKLREQYGKNKTIIFDQPSGGDPEIVTHSFNCGGEFFYCNSTQLFNSTWYDNSTWNENKNHTKNDTITLPCRIKQFINMWQEVGKAMYAPPIRGQIKCSSNITGLIITRDGGNNNSETFNNETFRPGGGNMKDNWRSELYKYKVVRIEPLGVAPTRAKRRVVQREKR

SEQ ID NO: 113 - DNA, 1685 bp, Human immunodeficiency virus type 1
agaaagagcagaagacagtggcaatgagagtgaaggggaccaagacgaattatcagcacttatggagatggggcatcatgctcctggggatattgatgatctgtagtgctacagaacaatggtgggtcacagtctattatggagtacctgtgtggagagatgcaaataccactctattttgtgcatcagattctaaagcatatgctacagaggtacataatgtttgggccacacatgcctgtgtacccacagaccccaacccacaagaaataaacttggaaaatgtaacagaagagtttaacatatggaaaaataacatggtagaacaaatgcatgaggatataatcagtttatgggatcaaagcctaaagccatgtgtaaaattaaccccactctgtgttactttaaattgcactgattacaatggtactcataccaatactactaataccactagtatttatggggaaaagatggaaataggagaagtaaagaaatgctctttcaatgctaccacaatcataagagataaggtggacaaagaagaagcacttttttataaacttgatatagtaccaatagatggtaataatgagactaacatagttaataatgggactaataatactagtaccaactataccagctataggctaataaattgtaacacctcagtcattacacaggcctgtccagaggtatcctttgagccaattcccatacattattgtgccccggctggttttgcgattctaaagtgtaaagagaaggcgttcaatggaagtgggccatgtaaaaatgtcagctcagtacaatgtacacatggaattaagccagtagtatcaactcaattgccgttaaatggcagtctagcagaagaagaagtagtaattagatctgaaaatttcacaaacaatgctaaaaccataatagtacagctgaaagaagctgtaaacattagttgtataaggcccaacaacaatacaagaaaaagtatacctataggaccagggagagcattttatgcaacaggagacataataggagatataggacaagcacattgtaaccttagtagaacaaattggaataaaactttacaacagatagctacaaaattaggagaaaagtttaataaaacaacaataatctttaatcaatcctcaggaggggacccagaaattgtaatgcacagttttatttgtggaggggaatttttctactgtaatacaacacaactgtttaatagtacttggaactgtactgagaatgggaattgtacactggttaccggtacttggcctgacaggccaaatagaactggagaaaatgacacaatcacactcccatgcagaataaaacaaatcataaacctgtggcaggaagtaggaaaagcaatgtatgcctctcccatccagggactaattaattgtacatcaaatattacagggctgctattaacaagagatggtggtacccatagtagacagaatgagaccttcagacctgaaggaggaaatatgaaagacaattggagaagtgaattatataaatataaagtagtaagaattgaaccattaggagtagcacccaccaaggcaaagagaagagtggtgcagagagaaaaaagagcagtgggattgggagctatgatccttgggttcttgggagcagcaggaagcact

SEQ ID NO: 114 - protein, 509 aa, Human immunodeficiency virus type 1
SATEQWWVTVYYGVPVWRDANTTLFCASDSKAYATEVHNVWATHACVPTDPNPQEINLENVTEEFNIWKNNMVEQMHEDIISLWDQSLKPCVKLTPLCVTLNCTDYNGTHTNTTNTTSIYGEKMEIGEVKKCSFNATTIIRDKVDKEEALFYKLDIVPIDGNNETNIVNNGTNNTSTNYTSYRLINCNTSVITQACPEVSFEPIPIHYCAPAGFAILKCKEKAFNGSGPCKNVSSVQCTHGIKPVVSTQLPLNGSLAEEEVVIRSENFTNNAKTIIVQLKEAVNISCIRPNNNTRKSIPIGPGRAFYATGDIIGDIGQAHCNLSRTNWNKTLQQIATKLGEKFNKTTIIFNQSSGGDPEIVMHSFICGGEFFYCNTTQLFNSTWNCTENGNCTLVTGTWPDRPNRTGENDTITLPCRIKQIINLWQEVGKAMYASPIQGLINCTSNITGLLLTRDGGTHSRQNETFRPEGGNMKDNWRSELYKYKVVRIEPLGVAPTKAKRRVVQREKR

SEQ ID NO: 115 - DNA, 1632 bp, Human immunodeficiency virus type 1
agaaagagcaaaacagtggcaatgaaagtgaaggggaccaggatgaattgtcagcgctggtggtggacatggggcacgatgctccttgggatgttgatgatctgtagtgctgcagaaaagttgtgggtcacagtctattatggggtacctgtgtggaaagaagcaaccaccactctattttgtgcatcagatgctaaagcatataagacagaaaagcataatgtctgggccacacatgcctgtgtacccacaaaccccaacccacaagaagtagtaatggaaaatgtaacagaatattttaacatgtggaaaaataacatggtagaacagatgcaggaggatataatcagtttatgggatcaaagcctaaagccatgtgtaaaattaaccccactctgtgtaactttaacttgtgtgaatattactaactgtaagaataatactaactgtaataatgatactaacagtaagaatgatactcttaaggaggagataggggaaataaaaaactgctctttcaacgtcaccacagccataagagataaggtgcagaaagaatatgcattatttcataaacttgatgtagtacaaatagataatgataatactagtagtaatacttctaagccttataggttgataagttgtaacacctcagtcattacacaggcctgtccaaaggtaacctttgagccaattcccatacattattgtgcctcggctggttttgcgattctaaagtgtaacaataagactttcaatggaacaggaccatgtacaaatgtcagcacagtacaatgtacacatggaattaggccagtagtatcaactcaactgttgttaaatgacagcctagcagaaaaagaggcaatagttagatctgaaaatttcacaaacaacgctaaaatcataatagtacagctaaatgaatctgtagagattaattgtacaagacccaacaacaatacaagaagaagtatacctataggaccagggaaagcattttttacatcagaaataataggagatataagaaaagcacactgtaacattagtggaacaaagtggaatgccactttgcataaaatagctataaaattaagagaacaatatggaaataaaacaatagtctttaatcaaccttcaggaggggacccagaagttgtaatgcacagttttaactgtggaggggaatttttctactgtgatacaacacaactgtttaatagtacttggtttaatagtacttggccaaatatcacacttgaagaaaatatcacactcccatgcaaaataaaacaatttataaacatgtggcaggaagtaggaaaagcaatgtatgcccctcccatcagaggacaaattaactgttcatcacagattacagggctgctattaacaagagatggtggtcagggtaacaatactaacaacgacactgagattttcagaccagggggaggagatatgagggacaattggagaagtgaattatacaaatataaagtagtaagaattgagccattgggagtagcacccaccaaggcaatgagaagagtggtgcagagagaaaaaagagcaataggactaggagcttttttccttgggttcttgggagcagcaggaagcact

SEQ ID NO: 116 - protein, 491 aa, Human immunodeficiency virus type 1
SAAEKLWVTVYYGVPVWKEATTTLFCASDAKAYKTEKHNVWATHACVPTNPNPQEVVMENVTEYFNMWKNNMVEQMQEDIISLWDQSLKPCVKLTPLCVTLTCVNITNCKNNTNCNNDTNSKNDTLKEEIGEIKNCSFNVTTAIRDKVQKEYALFHKLDVVQIDNDNTSSNTSKPYRLISCNTSVITQACPKVTFEPIPIHYCASAGFAILKCNNKTFNGTGPCTNVSTVQCTHGIRPVVSTQLLLNDSLAEKEAIVRSENFTNNAKIIIVQLNESVEINCTRPNNNTRRSIPIGPGKAFFTSEIIGDIRKAHCNISGTKWNATLHKIAIKLREQYGNKTIVFNQPSGGDPEVVMHSFNCGGEFFYCDTTQLFNSTWFNSTWPNITLEENITLPCKIKQFINMWQEVGKAMYAPPIRGQINCSSQITGLLLTRDGGQGNNTNNDTEIFRPGGGDMRDNWRSELYKYKVVRIEPLGVAPTKAMRRVVQREKR

SEQ ID NO: 117 - DNA, 1679 bp, Human immunodeficiency virus type 1
agaaagagcagaagacagtggcaatgagagtgatggggatcaggaagagttatcagcacttgtggaaagggggcaccttgctccttggaatattgatgatctgtagtgctgcagaacaattgtgggtcacagtctattatggggtacctgtgtggaaagatgcaaccaccactttattttgtgcatcagatgctaaagcatatgatacagaggtacacaatgtttgggccacacatgcctgtgtacccacagaccccaacccacaagaagtagtaatgggaaatgtgacagaatattttaacatgtggacaaataacatggtagaacagatgcatgaggatgtaatcagtttatgggatcaaagcctaaagccatgtgtaaaattaaccccactctgtgttactttaaattgcactaatttggtgaatactacctgtaatgggactactaacaataatactacctgtactgggactgctaacaatgatactaataccaatagtactaggtgggtgtatcaagcgatggcaggagaaataaaaaactgctctttcaatatcaccacaaacataagagataagataaaaaaagaatatgcactttttaatagacttgatatagtaccaatagatgatgagaataagaatactggcaatactactagctataggttgataagttgtaacacctcagtcattacacaggcctgtccaaaggtaacctttgaaccaattcccatacattattgtgccccggctggttttgcgattctcaagtgtaatgataagaagttcaatggaacaggaccatgtacaaatgtcagcacagtacaatgtacacatggaattaggccagtagtatcaactcaactactattaaatggcagtctagcagaagaagagacagtaattagatctagcaatttctcgaacaatgctaaaatcataatagtacagctgaatgaaactgtacgaattaattgtacaagacccaacaacaatacaagaagaagtatacatataggaccagggagagcattttatgcaacaggagacataataggagatataagacaagcacattgtaacattagtggagaagaatggaggagaactttaaaacggataactataaaattaggagaacaatttaataaaacaaaaataagctataaccaatcctcaggaggggacccagaaattgtaaggcacagttttaattgtcaaggggaatttttctactgtgatacatcaggactgtttaatagtacttgggtgaagaatgatacttggaatgagagtagtattagcaatggaactatcacactcccatgcagaataaaacaaattgtaaacatgtggcaggaagtaggaagagcaatgtatgcccctcctatcaaaggacaaattaattgtacatcgaatattacagggctgctactaacaagagatggtggtcagactaatagcaccaacaacgacactgagaccttcagacctacaggaggagatataagggacaattggaggagtgaattatataaatataaagtagtaaaaattgaaccattaggaatagcacccaccagggcaaaaagaagagtggtgcaaagagaaaaaagagcagtgggaacgatgggagcgttgttccttgggttcttgggagcagcaggaagcact

SEQ ID NO: 118 - protein, 506 aa, Human immunodeficiency virus type 1
SAAEQLWVTVYYGVPVWKDATTTLFCASDAKAYDTEVHNVWATHACVPTDPNPQEVVMGNVTEYFNMWTNNMVEQMHEDVISLWDQSLKPCVKLTPLCVTLNCTNLVNTTCNGTTNNNTTCTGTANNDTNTNSTRWVYQAMAGEIKNCSFNITTNIRDKIKKEYALFNRLDIVPIDDENKNTGNTTSYRLISCNTSVITQACPKVTFEPIPIHYCAPAGFAILKCNDKKFNGTGPCTNVSTVQCTHGIRPVVSTQLLLNGSLAEEETVIRSSNFSNNAKIIIVQLNETVRINCTRPNNNTRRSIHIGPGRAFYATGDIIGDIRQAHCNISGEEWRRTLKRITIKLGEQFNKTKISYNQSSGGDPEIVRHSFNCQGEFFYCDTSGLFNSTWVKNDTWNESSISNGTITLPCRIKQIVNMWQEVGRAMYAPPIKGQINCTSNITGLLLTRDGGQTNSTNNDTETFRPTGGDIRDNWRSELYKYKVVKIEPLGIAPTRAKRRVVQREKR

SEQ ID NO: 119 - DNA, 1589 bp, Human immunodeficiency virus type 1
agaaagagcagaagacagtggcaatgagagtgagggggatcatgaggaattacttgtggaaatggggcatcatgctccttgggatattgatgatctgtagtgctacagacaaattgtgggtcacagtctattatggggtgcctgtgtggaaagaagcatccaccactctattttgtgcatcagatgctaaagcctatgatacagaggtacataatgtttgggccacacatgcctgtgtacccacagatcccaatccacacgaattagaattggaaaatgtgacagaagattttaacatgtggaaaaatgacatggtcgaacagatgcatgaggatataatcagtttatgggatcaaagcctaaaaccatgtgtaaaattaaccccactctgtgttactttaaattgcagtgatgctttaacttgcaataggacatcaaatagcagtagtacttcaaattgcagtaactgggaaccgatagaagaaataaaaaattgctctttcaatattaccacaagcatagaaaataagatgcagaaaaagtctgcattttttgatgcccttgatgtagtacaaatagatgatactagttataggttgataaattgtaacacctcagtcattacacaggcctgtccaaagatatcctttgagccaattcccatacattattgtgccccggctggttttgcgcttctaaagtgtaaggatccgaaattcaatggaacagggccatgtaaatatgctagctcagtacagtgtacacatggaattaggccggtagtatcaactcaactgctgctaaatggcagtctagcagaagaagatatagtaattagatctgccaatttctcggacaacaccaaagccataatagtacaactaaaagaacctgtaataattaattgcacaagacccaacaacaatacaagacaaagtgtacatataggaccagggagcgcactttatacaacagatataataggagatataagaaaagcacattgtaacattagtagagcagactggactaaagctttaaaccagacagtcataaaattaagagaacaatttaagaataaaacaatagtctttaatcaatcctcaggaggggatccagaaattgtaatgcacacttttaattgtggaggggaatttttctattgtaattcaacaaaactgtttaatagtacttggaatgggactgaaccaggagagtcaaatgacactgtaatcatactcccatgcagaataaaacaaattataaatatgtggcaggaagtaggaaaagcaatgtatgcccctcccatcagaggacaaattagatgtacatcaaatattacagggctgctactaacaagagatgggggaaatgagaccactaaaaacgggactgagaccttcagacctggaggaggaaatatgaaggacaattggagaagtgaattatataaatataaagtggtaaaaattgaaccattaggagtagcacccaccaaggcaaaaagaagagtggtgcagagagaaaaaagagcaataggggcattcggagctatgttccttgggttcttgggagcagcaggaagcact

SEQ ID NO: 120 - protein, 478 aa, Human immunodeficiency virus type 1
SATDKLWVTVYYGVPVWKEASTTLFCASDAKAYDTEVHNVWATHACVPTDPNPHELELENVTEDFNMWKNDMVEQMHEDIISLWDQSLKPCVKLTPLCVTLNCSDALTCNRTSNSSSTSNCSNWEPIEEIKNCSFNITTSIENKMQKKSAFFDALDVVQIDDTSYRLINCNTSVITQACPKISFEPIPIHYCAPAGFALLKCKDPKFNGTGPCKYASSVQCTHGIRPVVSTQLLLNGSLAEEDIVIRSANFSDNTKAIIVQLKEPVIINCTRPNNNTRQSVHIGPGSALYTTDIIGDIRKAHCNISRADWTKALNQTVIKLREQFKNKTIVFNQSSGGDPEIVMHTFNCGGEFFYCNSTKLFNSTWNGTEPGESNDTVIILPCRIKQIINMWQEVGKAMYAPPIRGQIRCTSNITGLLLTRDGGNETTKNGTETFRPGGGNMKDNWRSELYKYKVVKIEPLGVAPTKAKRRVVQREKR

SEQ ID NO: 121 - DNA, 1616 bp, Human immunodeficiency virus type 1
agaaagagcagaagacagtggcaatgaaagtgaaggggatcaggaagaattgtcagcgcttgtggagatggggcacgatgctccttgggatgttaatgatatgtagtgctgcagagcaattgtgggtcacagtctattatggggtacctgtgtggagagaagcaaacaccactctattctgtgcctcagatgctaaagcacaggttgcagaggcacataatgtatgggccacacatgcctgtgtacccacagaccctagcccacaagaagtagtaatggaaaatgtgacagaaaattttaacatgtggaaaaataacatggtagaacagatgcatgaggatataatcagtttatgggatcaaagtctaaagccatgtgtgaaattaaccccactctgcgttactttaaattgcactaatgtgggttgcactggtaatactactggacccaattgtacttctttgactgatcataatagtaatcttacttggggaatggagaaaggagaaataaaaaattgctctttcaatgtcaccagtataacaaataagatgcagaaagaatatgcacttttttataaacttgatgtaatgccaatggatagtacagataatacaacgtatacactgataaattgtaacccctcagtcattacacaggcctgtccaaaggtatcttttgaacccattcctatacattattgtaccccggctggttttgcgattctaaagtgtaatgataagacattcaatggatcaggaccatgtacaaatgtcagtacagtactatgtacacatggaattaggccagtagtgtcaactcaactactgttaaatggcagtctagcagaagaggaggtaatagtcaggtccgagaatttctcggacaatactaaaatcataatagtacagctgaataaaactgtagaaattaattgtacaagacccaataacaatacaagaaaaagtatacatatagcaccaggaaaagcattctatgcaacaggtgatataataggagatataagacaagcacattgtaacatcagtgaaacaaaatgggtgaacactttaaaacaggtagttacaaaattaagggaacaatatgggaataaaacaatagcctttaatcaatcctcaggaggggatccagaaattgtaacgcatagttttaattgtggaggagaatttttctactgtaatacatcacggctgtttaatagtaattggactgggaatggaacgactgagtcaggaaatagcacaatcatacttccatgcagaataaaacaaattataaacagatggcaggaagtaggaaaagcaatgtatgccaatcccattagtggaccaatcaactgttcatcaaacattacagggctgctattaacaagagatggtggtaaagtgaccaatgacaccaccgagaccttcagaccttggggtggagatatgagggacaattggagaagtgaactatataaatataaagtagtaagaattgagccattaggactagcacccaccagggcaaagagaagagtggtgcagagagaaaagagagcaataacattaggagctatgttccttgggttcttgggagcagcaggaagcact

SEQ ID NO: 122 - protein, 486 aa, Human immunodeficiency virus type 1
SAAEQLWVTVYYGVPVWREANTTLFCASDAKAQVAEAHNVWATHACVPTDPSPQEVVMENVTENFNMWKNNMVEQMHEDIISLWDQSLKPCVKLTPLCVTLNCTNVGCTGNTTGPNCTSLTDHNSNLTWGMEKGEIKNCSFNVTSITNKMQKEYALFYKLDVMPMDSTDNTTYTLINCNPSVITQACPKVSFEPIPIHYCTPAGFAILKCNDKTFNGSGPCTNVSTVLCTHGIRPVVSTQLLLNGSLAEEEVIVRSENFSDNTKIIIVQLNKTVEINCTRPNNNTRKSIHIAPGKAFYATGDIIGDIRQAHCNISETKWVNTLKQVVTKLREQYGNKTIAFNQSSGGDPEIVTHSFNCGGEFFYCNTSRLFNSNWTGNGTTESGNSTIILPCRIKQIINRWQEVGKAMYANPISGPINCSSNITGLLLTRDGGKVTNDTTETFRPWGGDMRDNWRSELYKYKVVRIEPLGLAPTRAKRRVVQREKR

SEQ ID NO: 123 - DNA, 1610 bp, Human immunodeficiency virus type 1
agaaagagcagaagacagtggcaatgagagtgaaggagatcaggaagaattggcagcgcttgtggagatggggcatgatgctccttgggatgttgatgatcagtagtgctgaagaagatttgtgggtcacagtctattatggggtacctgtgtggaaagaagcagagaccactttattttgtgcatcagatgctaaagcatataacacagaggcacataatgtgtgggccacacatgcctgtgtaccaacagaccctagcccacaagaagtattattggtaaatgtgacagaaaattataacatgtggaaaaataacatggtagaacagatgcatgaggatataattagtttatgggatcaaagcctaaagccatgtgtaaaattaaccccgctttgtgttactttaaattgcactaatgtgaattgcactcatgagaatggtaccactaccgagtgcggtaataatgggatacagatggagaaaggagaaatgaaaaactgctctttcaatattaccacaagcataaaaaataagatgcagaaagaatatgcacttttgtataaactagatttagcatcaataggtaatgataatacaagctatactttgataagttgtaacacctcagtcattacacaggcctgtccaaagatatcctttgaaccaattccaatacattattgtgccccggctggttttgcgattctaaaatgtaatgataagaacttcaagggaacaggatcatgtaaaaatgtcagcacagtacaatgtacacatggaattaagccagtagtgtcaactcaattgttgttaaatggcactttagcagaaacagaggtagtaattagatctgaaaatatcacagacaatgctaaaaccataatagtacaactgaaggaccctgtaaaaattaattgtacaagacctggcaacaatacagcaagaagcatacatatgggaccggggagagcattttctgcaacaggacaaataataggaaatataagacaagcacattgtaaccttagtagaacagaatgggatgacactttaaaaaagatagctaagaaattaggagaacaatttaggaataaaagtatagcctttaatcaatcctcaggaggggacccagaaattgtaatgcacagttttaattgtggaggggaatttttttactgtaatacatcacagctgtttaatagtacttggtggaacaatggtactaggaatgatgctgcaaggtcaaatagcactgaacctatcacactccggtgcagtataaagcaaattataaacagatggcaggaagtaggaaaagcaatgtatgcccctcccatcaggggaaacgttacatgtaactcaagtattacagggctactcttaataagagatggtgggaacagtaatgagtctactgagaccttcagacctcagggaggaaatatgaaggacaattggagaagtgaattatacaaatataaagtagtaaaaattgagccattaggagtagcacccaccaaggcaaagagaagagtggtgcagagagaaaaaagagcagtgggactaggagctgtgttccttgggttcttgggagcagcaggaagcact

SEQ ID NO: 124 - protein, 484 aa, Human immunodeficiency virus type 1
SAEEDLWVTVYYGVPVWKEAETTLFCASDAKAYNTEAHNVWATHACVPTDPSPQEVLLVNVTENYNMWKNNMVEQMHEDIISLWDQSLKPCVKLTPLCVTLNCTNVNCTHENGTTTECGNNGIQMEKGEMKNCSFNITTSIKNKMQKEYALLYKLDLASIGNDNTSYTLISCNTSVITQACPKISFEPIPIHYCAPAGFAILKCNDKNFKGTGSCKNVSTVQCTHGIKPVVSTQLLLNGTLAETEVVIRSENITDNAKTIIVQLKDPVKINCTRPGNNTARSIHMGPGRAFSATGQIIGNIRQAHCNLSRTEWDDTLKKIAKKLGEQFRNKSIAFNQSSGGDPEIVMHSFNCGGEFFYCNTSQLFNSTWWNNGTRNDAARSNSTEPITLRCSIKQIINRWQEVGKAMYAPPIRGNVTCNSSITGLLLIRDGGNSNESTETFRPQGGNMKDNWRSELYKYKVVKIEPLGVAPTKAKRRVVQREKR

SEQ ID NO: 125 - DNA, 1619 bp, Human immunodeficiency virus type 1
agaaagagcagaagacagtggcaatgagagtgatggggataaagaagaattactggtggagatggggcccgatgctccttgggatattgatgacctatagtgcagcagaattttgggtcacagtctactatggagtgccagtgtggaaagaaacaaccaccactctattttgtgcatcagatgccaaagcatatgatacagaggcacataatgtttgggccacacatgcctgtgtacccacagaccccaacccacaagaagtagtattggaaaaggtgacagaagagtttaacatgtggaaaaatagcatggtagaacagatgcatgaggatataatcagtttatgggatcaaagtctaaagccatgtgtaaaactaaccccactctgtgttactttaagttgcactgattgtaatggtactagccctgagtgtgcgaagaatgctagtactactaccactagtagtaagggattgatagataaaggggaaataaaaaactgctctttcaatgccaccacacacataatggataaggtgcagaaagaatatgcattattttataacactgatttagtacaaatagagggtgagaaatctgataataatactagatataggttaataagttgtaacacctcagtcattaaacaggcctgtccaaaggtatcttttgagccaattcccatacattattgtgccccggctggttttgcgattctaaagtgtaaagataagaatttcaatggaacaggaaaatgttacaatgtcagcacagtacaatgtacacatggaattaggccagtaatgtcaactcaactgctgttaaatggcagcctagcagaagaagaaatagtaattagatctgccaatttctcgaacaatgctaaaaccataatagtacatctgaatgaatctgtagaaattaactgcacaagacccaacaacgatacaaggaaaagtataaatataggaccagggagagcatggtatgcagcaggagaaataataggaaatataagaaaagcatattgtaacattagcagagcaaaatggaacaacactttaaaacatgtagttgaaaaactaagaaaacaatttggaaataaaacaataaactttacacaacacgcaggaggggacctagaaattgtgacgcatagttttaattgtggaggggaattcttctactgcaacacaacacagctgtttaatagtacttggcctaagaatggtacttggaatggtactggtagtgacattatcacactcccatgcaaaataaaacagattataaacatgtggcaggaggtaggaaaagcaatgtatgcccctcccatcagcggactaattagatgttcatcaaatattacagggctgctattaacaagagatggtggtaagggtaatggcacaaatgatacagagatcttcagaccaggaggaggagatatgagggacaattggagaagtgaattatataaatataaagtagtagaaattgagccaataggactagcacccaccaaggcaaagagaagagtggtgcagagagaaaaaagagcagtgggaacgctgggagctatgttccttgggttcttgggagcagcaggaagcact

SEQ ID NO: 126 - protein, 488 aa, Human immunodeficiency virus type 1
SAAEFWVTVYYGVPVWKETTTTLFCASDAKAYDTEAHNVWATHACVPTDPNPQEVVLEKVTEEFNMWKNSMVEQMHEDIISLWDQSLKPCVKLTPLCVTLSCTDCNGTSPECAKNASTTTTSSKGLIDKGEIKNCSFNATTHIMDKVQKEYALFYNTDLVQIEGEKSDNNTRYRLISCNTSVIKQACPKVSFEPIPIHYCAPAGFAILKCKDKNFNGTGKCYNVSTVQCTHGIRPVMSTQLLLNGSLAEEEIVIRSANFSNNAKTIIVHLNESVEINCTRPNNDTRKSINIGPGRAWYAAGEIIGNIRKAYCNISRAKWNNTLKHVVEKLRKQFGNKTINFTQHAGGDLEIVTHSFNCGGEFFYCNTTQLFNSTWPKNGTWNGTGSDIITLPCKIKQIINMWQEVGKAMYAPPISGLIRCSSNITGLLLTRDGGKGNGTNDTEIFRPGGGDMRDNWRSELYKYKVVEIEPIGLAPTKAKRRVVQREKR

SEQ ID NO: 127 - DNA, 1613 bp, Human immunodeficiency virus type 1
agaaagagcagaagacagtggcaatgagagcgaaggggatcaggaagagttgtcaacacttatggagatggggcaccatgctccttgggatgttgatgatttgtagtgctgcagaaaacttgtgggtcacagtctactatggggtacctgtgtggaaagaagcaaccaccactctattttgtgcatcgaatgctaaagcatatgagacagaggtgcataatgtttgggccacacatgcctgtgtacccacagaccccaacccacaagaagtagtattgggaaatgtgacagaaaattttaacatgtggaaaaataacatggtagaacagatgcatgaggatgtaattagtttgtgggaccaaagcttaaagccatgtgtaaaattgaccccactctgtgttactttacattgcactgattgtgagaatactattactggggggaataatactaatagtaaatgcaatgaggataaggggaatactactgccactatattgatagagaaaggagagatgaaaaactgctcttttaatgtcaccacagacctaagagataagatgcagaaagaatatgcacttgatgtagtaccattagacagtactaataccagctataagttagtaagttgtaacacctcagtcattacacaggcctgtccaaaggtatcttttgagccaattccaatacatttctgtgccccagctggttttgcgattctaaagtgtaacaataaaacgtttgatggaaaaggaccatgtacaaatgtcagtacagtgcgatgtacacatggaattaaaccagtagtgtcaactcaactgctgttaaatggcagtctagcagaagaagagatagtgattagatctgaaaatttctcgaacaatgctaaaaccataatagtacagctaaataaaactgtagaaattaattgtacaagacccaacaacaacacaagcaaaggtatacatatgggaccagggagggcattttatgcaacaggaagaatagtaggagatataagacaagcacattgtaacattagtaacgcagattggacaaatactttaaaacaggtagctaggaaattaagggaacaatatgtgaataaaacaatagcctttaagccaccctcaggaggggacccagaagttgtactgcacacttttaattgtagaggggaatttttctactgtaatttatcaagaatgtttaatagtagttttaattcaacacaactgtctaattattcagaagatactgggaccatcacagtcccatgcagaataaaacaatttataaacatgtggcaggaagtaggaaaagcaatgtatgcccctcccatcagaggagaaattaattgttcatcaaagattacaggattgttattaacaagagacggtggcaatagcaatgggactgagattttcagacctggaggaggagatatgagggacaattggagaagtgaattatacaaatataaagtagtaagaattgaaccattaggattagcacccaccaaggcaaagagaagagtggtgcagagagaaaaaagagcagcagtgacaatgggagcaatgttccctgggttcttgggagcagcaggaagcact

SEQ ID NO: 128 - protein, 484 aa, Human immunodeficiency virus type 1
SAAENLWVTVYYGVPVWKEATTTLFCASNAKAYETEVHNVWATHACVPTDPNPQEVVLGNVTENFNMWKNNMVEQMHEDVISLWDQSLKPCVKLTPLCVTLHCTDCENTITGGNNTNSKCNEDKGNTTATILIEKGEMKNCSFNVTTDLRDKMQKEYALDVVPLDSTNTSYKLVSCNTSVITQACPKVSFEPIPIHFCAPAGFAILKCNNKTFDGKGPCTNVSTVRCTHGIKPVVSTQLLLNGSLAEEEIVIRSENFSNNAKTIIVQLNKTVEINCTRPNNNTSKGIHMGPGRAFYATGRIVGDIRQAHCNISNADWTNTLKQVARKLREQYVNKTIAFKPPSGGDPEVVLHTFNCRGEFFYCNLSRMFNSSFNSTQLSNYSEDTGTITVPCRIKQFINMWQEVGKAMYAPPIRGEINCSSKITGLLLTRDGGNSNGTEIFRPGGGDMRDNWRSELYKYKVVRIEPLGLAPTKAKRRVVQREKR

SEQ ID NO: 129 - DNA, 1640 bp, Human immunodeficiency virus type 1
agaaagagcagaagacagtggcaatgagagtgaaggggatcaggaagaattatcagcacttatggagatggggcaccgtgctccttgggatgttgatgatctgtagtgctgtagaacaattgtgggtcacagtctattatggggtacctgtgtggaaagaagcaaccaccactttattttgtgcatcagatgctaaagcatatgacacagaggcacataatgtctgggccacacatgcctgtgtacctacagaccctaacccacaagaagtagtattggaaaatgtgacagaagattctaacatgtggaaaaataacatggtagaacagatgcatgaggatataatcagtttatgggatcaaagcctaaagccatgtgtaaaattaaccccactctgtgttactctaaattgcactgatttcaattgtactagttccagcaatactactaatagcacttgcattggtacccatcggactactaataccgatggtagggagaaattggaaatggaggtaggagaaataaaaaactgctctttcaatgtcaccacaagcataaggaataaggtacagaaagaatatgcacttttttataaacttgatgtaatgccaatagatagtacgagctatacattgatacattgcaacacttcaaccattacacaggcctgtccaaaggtatcctttgaaccaattcctatacattattgtgccccggctggttttgcgattctaaagtgtaacaataagacgttcagtggaaaaggaccatgtaaaaatgtcagcacagttcaatgtacacatggaattaggccagtagtgtcaactcaactgctgttaaatggtagtctagcagaagaagagatagtaattaggtctgacaatttctcggacaatgctaaaatcataatagtacacctaaataaatctatagaaattaattgtacaagacccaacaataatacaagaaaaagaatatcgatggggccgggaagagtatattatacaacaggacaaataataggagatataagaaaagcacattgtaatattagtggagaagaatggaatagaacgttaaaagggatagttataaaattaagagaacaatttgggaagaataaaacaatcatctttgatagatcctcaggaggggacctagaaattgaaatgcatagttttaattgtggaggagagttcttctactgtaatacaacaaaactatttaatagtgcttggaatgagtcaggttacaatgggacaaattctaatggaactattacactcccatgcagaataagacaaattgtaaacaggtggcaggaagtaggaaaagcaatgtatgcccctcccatcacaggacaaattaggtgttcatcaaatattacaggactaatattaacaagagatggtggtaacagtagcaatagtagtaatgtgaatgagaccttcagacctacaggaggagatatgagggacaattggagaagtgaattatataaatataaagtaatacgaattgagccaataggagtagcacccaccaaggcaaagagaagagtggtgcagagagagaaaagagcagtgggaacgctaggagctatgttccttgggttcttgggagcagcaggaagcact

SEQ ID NO: 130 - protein, 493 aa, Human immunodeficiency virus type 1
SAVEQLWVTVYYGVPVWKEATTTLFCASDAKAYDTEAHNVWATHACVPTDPNPQEVVLENVTEDSNMWKNNMVEQMHEDIISLWDQSLKPCVKLTPLCVTLNCTDFNCTSSSNTTNSTCIGTHRTTNTDGREKLEMEVGEIKNCSFNVTTSIRNKVQKEYALFYKLDVMPIDSTSYTLIHCNTSTITQACPKVSFEPIPIHYCAPAGFAILKCNNKTFSGKGPCKNVSTVQCTHGIRPVVSTQLLLNGSLAEEEIVIRSDNFSDNAKIIIVHLNKSIEINCTRPNNNTRKRISMGPGRVYYTTGQIIGDIRKAHCNISGEEWNRTLKGIVIKLREQFGKNKTIIFDRSSGGDLEIEMHSFNCGGEFFYCNTTKLFNSAWNESGYNGTNSNGTITLPCRIRQIVNRWQEVGKAMYAPPITGQIRCSSNITGLILTRDGGNSSNSSNVNETFRPTGGDMRDNWRSELYKYKVIRIEPIGVAPTKAKRRVVQREKR

SEQ ID NO: 131 - DNA, 1664 bp, Human immunodeficiency virus type 1
agaaagagcagaagacagtggcaatgaaagcgaaggagatgaagaagcattggcagcacttgtggaaagggggcatcatgctccttgggatgttaatgatctgtagtgctgcaccaaacttgtgggtcacagtctattatggggtacctgtgtggaaagaagcaaccaccactctattttgtgcatcagatgctaaagcatacaaaacagaggctcataatgtctgggccacacatgcctgtgtacccacagaccccaacccacaagaagtagtattggaaaatgtgacagaaaattttaacatgtggaaaaataacatggtagaacagatgcatgaggatataattagtctatgggatcaaagcctaaagccatgtgtaaaattaaccccactctgtgttactttaaattgcactaagttaaataattgcactgagttgcagaatgatactacgaatagtactacgttggagaatggtacttattgcattaaggtggagaataaaactaatataagggaagaaatgacaaattgctctttcaatattaccacaagtgtaagagataaggtgcataaacaatatgctctcttctatagatttgatttagtaccaatagaggatgaaaataagaatactagctctaataatagctttagattgataaattgtaatacctcaatcattacacagtcctgtccaaaggtaacctttgagccaattcccatacattattgtaccccagctggttttgcgattctaaagtgtaatgataagaagttcaatggaaaagggccatgtacaaatgtcagcacagtacaatgtacacatggaattaaaccagtagtgtcaactcaactgctgttaaatggcagtctagcagaagaagaggtagtaattagatctgaaaacttcacaaacaatgctaaaaccataatagtacagctgaacgagactgtagaaattaattgtacaagacctagcaacaatacaagaaaaagtataactataggaccagggagagcattttttacaacaggggatgtaataggaaatataaggcaagcatattgtaacgttagtagggcaaaatggaataacactttaagacagatagttacaaaactaagagaacaatttgagaataaaacaataatttttaagtcatcctcgggaggggacccagaaattgtaactcacacttttaattgtggaggagaatttttctactgtaatacaacaccactgtttaatagtacctgggataatagtacctgggattggaataatactgaagagtcaaacagcactcccattgtactcacatgcagaataaaacaaattgtaaatatgtggcaggaggtaggaaaagcaatgtatgcccctcccatcagaggacaaatttggtgttcatcaaatattacaggactgctattaacaagagatggtggaataaataatacgggaaatgagaccttcagacctgcaggaggagatatgagggacaattggagaagtgaatcatataaatataaagtagtaagaattgaaccattaggagtggcacccaccaaggcaaagagaagagtggtgcagagagaaaaaagagcagtgggaacaataggagctatgttccttgggttcttgggagcagcaggaagcact

SEQ ID NO: 132 - protein, 501 aa, Human immunodeficiency virus type 1
SAAPNLWVTVYYGVPVWKEATTTLFCASDAKAYKTEAHNVWATHACVPTDPNPQEVVLENVTENFNMWKNNMVEQMHEDIISLWDQSLKPCVKLTPLCVTLNCTKLNNCTELQNDTTNSTTLENGTYCIKVENKTNIREEMTNCSFNITTSVRDKVHKQYALFYRFDLVPIEDENKNTSSNNSFRLINCNTSIITQSCPKVTFEPIPIHYCTPAGFAILKCNDKKFNGKGPCTNVSTVQCTHGIKPVVSTQLLLNGSLAEEEVVIRSENFTNNAKTIIVQLNETVEINCTRPSNNTRKSITIGPGRAFFTTGDVIGNIRQAYCNVSRAKWNNTLRQIVTKLREQFENKTIIFKSSSGGDPEIVTHTFNCGGEFFYCNTTPLFNSTWDNSTWDWNNTEESNSTPIVLTCRIKQIVNMWQEVGKAMYAPPIRGQIWCSSNITGLLLTRDGGINNTGNETFRPAGGDMRDNWRSESYKYKVVRIEPLGVAPTKAKRRVVQREKR

SEQ ID NO: 133 - DNA, 1640 bp, Human immunodeficiency virus type 1
agaaagagcagaagacagtggcaatgaaagcgaaggagaccaggaagaactatcagcgcttgtggagatggggcacgatgctccttgggatgttgatgatctgtagtgctacagaaaaattgtgggtcacagtctactatggggtacctgtgtggaaagaagcaactaccactctattttgtgcatcagatgctaaagcatatgatacggaggtacataatgtttgggccacacatgcctgtgtacccacagaccccaacccacaagaagtagtattggtaaatgtaacagaaaagtttaacatgtggaaaaataacatggtagaacaaatgcatgaggacataatcaatctatgggatcaaagcctaaagccatgtgtaaaattaaccccgctctgtattactttaaattgctctgatgttagaaattgcactgagcaggggaatgatactgccgctagtacttgtattgattggaagactaatggtagtgagaaagtgatggagaaaggagaaataaaaaactgctctttcaatattacaacaaacataagggacaaggtgaaggaagagtatgcacttttttataaaattgatatagcaccaatagataatgatactactagctataggttgataaattgtaacacctcagtcattacacaggcctgtccaaaggtatcctttgagccaattcccatacattattgtgccccggctggttttgcgattctaaaatgtggagataagaagttcaatggaacaggactatgtaaaaatgtcagcacagtacaatgtacacatggaattaggccagtagtgtcaactcaactgctgttaaacggcagtctagcagaagaagatgtaataattagatctgccaatttcacagacaatgctaaaaacataatagtacagctgaaggaatctgtagaaattacttgtataagacccaacaatacaagaaaaagtattcatataggaccagggaaaacattttttacaacagaaataataggaaatataagacaagcatattgtacccttgatggaacaaaatggaataacactttagcacagatagttgaacaattaaggggacaatttggaaataaaacaatagactttaagcaaccctcaggaggggacccagaagttataatgcacaagtttaattgtggaggggaatttttctactgtaattcaacacaactgtttaatagtacttggccactaaatggtactagatcaggtggcactgaaggaagcactgaaggaaatatcacactcccatgtaaaataaaacaaattataaacaggtggcaggaagtaggaaaagcaatgtatgcccctcccatcaaaggaataattagatgttcatcgaatattacagggctgatattaacaagagatggtggggagggcaagaacgataccaacagtaccgagatcttcagacctggaggaggagatatgagggacaattggagaagtgaattatataaatataaagtagtacgaattgaaccattaggagtagcacccaccaaggcaaagagaagagtggtgcagagagaaaaaagagcagtggggatgctaggagctatgttccttgggttcttgggagcagcaggaagcact

SEQ ID NO: 134 - protein, 493 aa, Human immunodeficiency virus type 1
SATEKLWVTVYYGVPVWKEATTTLFCASDAKAYDTEVHNVWATHACVPTDPNPQEVVLVNVTEKFNMWKNNMVEQMHEDIINLWDQSLKPCVKLTPLCITLNCSDVRNCTEQGNDTAASTCIDWKTNGSEKVMEKGEIKNCSFNITTNIRDKVKEEYALFYKIDIAPIDNDTTSYRLINCNTSVITQACPKVSFEPIPIHYCAPAGFAILKCGDKKFNGTGLCKNVSTVQCTHGIRPVVSTQLLLNGSLAEEDVIIRSANFTDNAKNIIVQLKESVEITCIRPNNTRKSIHIGPGKTFFTTEIIGNIRQAYCTLDGTKWNNTLAQIVEQLRGQFGNKTIDFKQPSGGDPEVIMHKFNCGGEFFYCNSTQLFNSTWPLNGTRSGGTEGSTEGNITLPCKIKQIINRWQEVGKAMYAPPIKGIIRCSSNITGLILTRDGGEGKNDTNSTEIFRPGGGDMRDNWRSELYKYKVVRIEPLGVAPTKAKRRVVQREKR

SEQ ID NO: 135 - DNA, 1672 bp, Human immunodeficiency virus type 1
agaagagcagaagacagtggcaatgaaagtgaaggagaccaggaagaattatcagaacttatggagatggggcatcttgctccttgggatattaatgatctgtagtgctgcagaaaaattgtgggtaacagtctattatggggtacctgtgtggagagaagcaaacaccactttattttgtgcatcagatgctaaagcatatgatacagaagtacataatgtctgggccacacatgcctgtgtgcccacagaccccaacccacaagaagtagtattgggaaatgtgacagaatattttaatatgtggaaaaataacatggtagaacagatgcatgaggatataatcagtttatgggatcaaagcctaaagccatgtgtaaaattaactccactctgtgttactttaaattgcactgatgcagtctgtacttcaaattgcactaattccactggtacgtccactcctattcccaccactgttagcagtgaggacaaaggagaaataaaaaactgctctttcaatgtcaccacaagcataaaagataggatacagagagaatatgcaactttttataagcttgatgtagtaccaatagatgatgatgataatactagagatgataataatactagtaataataatactagtaaccctagtaagactctctataggttgataaattgtaacacctcagcccttacacaggcctgtccaaaggtatcctttgaaccaattcccatacattattgtgccccagctggttttgcgattctaaagtgtaacaataagacattcgatggatcgggaccatgtacaaatgtcagcacagtacaatgtacacatggaattaggccagtagtgtcaactcaattgctgttaaatggcagtctagcagaaggagatatagtaatcagatctgaaaatttctcgaacaatgctaaaaccataatagtacagctgaaggaatctataagcattaattgtacaagacccaacaacaatacaagaaaaagtatacatataggacaaggaagggcattttatacaacaggagatataataggagatataagaaaagcacattgtaacgttagtagagaaggttggaataacgctgtaaaccgactagttgaaaaattaaaagaacaatttggaaaaagaaaaacaataaaatttaagccatcctcaggaggggacccagagattgtaatgcacatgtttaattgtggaggggagtttttctactgtaatacatcaaaactgtttaatgttgttaatgatacttggatgggatcaaatgacactggagaaatcgagctcccatgtagaataaaacaaatcgtaagcatgtggcaggaagtaggaaaagcaatgtatgcccctcccatcagaggacaaattagatgttcatcagatatcacagggctgctattaacaagagatggtggtaaggatgacaacaacacaattggaaatgaaaccttcagacctggaggaggagatatgagggacaattggagaagtgaattatataaatataaagtagtaaaaattcaaccactaggaatagcacccaccaaggcaaagagaagagtggtgcagagagaaaaaagagcagtgggagcactaggagctatgttccttgggttcttgggagcagcaggaagcact

SEQ ID NO: 136 - protein, 504 aa, Human immunodeficiency virus type 1
SAAEKLWVTVYYGVPVWREANTTLFCASDAKAYDTEVHNVWATHACVPTDPNPQEVVLGNVTEYFNMWKNNMVEQMHEDIISLWDQSLKPCVKLTPLCVTLNCTDAVCTSNCTNSTGTSTPIPTTVSSEDKGEIKNCSFNVTTSIKDRIQREYATFYKLDVVPIDDDDNTRDDNNTSNNNTSNPSKTLYRLINCNTSALTQACPKVSFEPIPIHYCAPAGFAILKCNNKTFDGSGPCTNVSTVQCTHGIRPVVSTQLLLNGSLAEGDIVIRSENFSNNAKTIIVQLKESISINCTRPNNNTRKSIHIGQGRAFYTTGDIIGDIRKAHCNVSREGWNNAVNRLVEKLKEQFGKRKTIKFKPSSGGDPEIVMHMFNCGGEFFYCNTSKLFNVVNDTWMGSNDTGEIELPCRIKQIVSMWQEVGKAMYAPPIRGQIRCSSDITGLLLTRDGGKDDNNTIGNETFRPGGGDMRDNWRSELYKYKVVKIQPLGIAPTKAKRRVVQREKR

SEQ ID NO: 137 - DNA, 1533 bp, Human immunodeficiency virus type 1
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaaga

SEQ ID NO: 138 - protein, 511 aa, Human immunodeficiency virus type 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 139 - protein, 511 aa, Artificial
  HIV-1 HXB2 gp120 with Cys mutations of U-101c1
MRVKEKYQHLWRWGWRWGTMLLGMLMICSATEKLWVTVYYGVPVWKEATTTLFRASDAKAYDTEVHNVWATHACVPTDPNPQEVVLVNVTENFNMWKNDMVEQMHEDIISLWDQSLKPCVKLTPLRVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 140 - protein, 511 aa, Artificial
  HIV-1 HXB2 gp120 with Cys mutations of U-178c13
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKRTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKYNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

(19) United States
(12) Patent Application Publication
(10) Pub. No.: US 2005/0025779 A1
(43) Pub. Date: Feb. 3, 2005

(54) HIV-1 envelope glycoproteins having unusual disulfide structure

(75) Inventors: Phillip W. Berman, Portola Valley, CA (US); David V. Jobes, Redwood City, CA (US)

Correspondence Address: QUINE INTELLECTUAL PROPERTY LAW GROUP, P.C., P O BOX 458, ALAMEDA, CA, 94501, US

(73) Assignee: VaxGen, Inc.

(21) Appl. No.: 10/866,527

(22) Filed: Jun. 10, 2004

Related U.S. Application Data
(60) Provisional application No. 60/477,815, filed on Jun. 12, 2003.

(51) Int. Cl. A61K 39/21

(52) U.S. Cl. 424/188.1; 424/208.1

(57) ABSTRACT

The present invention provides HIV-1 envelope glycoproteins having unusual disulfide structure. In particular, the invention includes gp120 polypeptides, and polynucleotides encoding such polypeptides, as well as related vectors, host cells, and expression methods. The invention also encompasses immunogenic compositions containing gp120 polypeptides or polynucleotides and their use in eliciting a gp120-specific immune response. gp120 polypeptides and polynucleotides of the invention are also useful in diagnostic methods of the invention.

### CROSS-REFERENCE TO RELATED APPLICATIONS

This application claims the benefit of U.S. Provisional Application No. 60/477,815, filed on Jun. 12, 2003, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under Small Business Research Grant (SBIR) No. 4 R44 AI052624-02. The government may have certain rights in this invention.

### FIELD OF THE INVENTION

The present invention relates generally to the area of immunogenic compositions useful for eliciting or measuring immune responses against human immunodeficiency virus type 1(HIV-1) envelope glycoproteins: gp120 and/or gp160. In particular, the invention relates to immunogenic compositions comprising a polypeptide comprising a gp120 sequence, and/or a polynucleotide (RNA or DNA) encoding such a polypeptide, wherein the compositions elicit an immune response useful in the prevention or treatment of HIV-1 infection and/or disease (AIDS).

### BACKGROUND OF THE INVENTION

Acquired immunodeficiency syndrome (AIDS) is caused by a retrovirus identified as the human immunodeficiency virus (HIV). There have been intense efforts to develop a vaccine that induces a protective immune response based on induction of antibodies or cellular responses. Recent efforts have used subunit vaccines where an HIV protein, rather than attenuated or killed virus, is used as the immunogen in the vaccine for safety reasons. Subunit vaccines generally include gp120, the portion of the HIV envelope protein that is displayed on the surface of the virion and virus-infected cells. In this regard, gp120 mediates HIV-1 infection by free virions or by cell-to-cell fusion. In both circumstances, gp120 initiates HIV-1 infection by binding to two cellular receptors: one being CD4 and the other being a chemokine receptor (typically CCR5 or CXCR4). Distinct high-affinity binding sites for CD4 and chemokine receptors have been located on the surface of the gp120 molecule.

The HIV envelope protein has been extensively described, and the amino acid and nucleotide sequences for HIV envelope from a large number of HIV strains are known. The gp120 molecule consists of a polypeptide core of 60,000 daltons, which is extensively modified by N-linked glycosylation to increase the apparent molecular weight of the molecule to 120,000 daltons. The mature HIV-1 envelope proteins, gp120 and gp41 are both derived from a common precursor, gp160. The gp160 precursor contains an amino-terminal signal sequence that directs the protein to be synthesized on membrane-bound ribosomes and ensures translocation of nascent polypeptides into the lumen of the endoplasmic reticulum (secretory pathway). In the endoplasmic reticulum, the signal sequence is removed by a signal peptidase, and the protein acquires the “simple” high-mannose type of N-linked carbohydrate in a cotranslational process. The carbohydrate-containing protein is then transported to the Golgi Apparatus where much of the high-mannose carbohydrate is converted to “complex” sialic acid-containing carbohydrate. In addition gp160, is converted to a gp120/gp41 complex by proteolysis by furin or a furin-like peptidase at a conserved glycoprotein processing site. The gp120/gp41 complex is then exported to the cell surface where it is thought to form trimer structures. In cellular or virion membranes, gp120 occurs as a peripheral membrane protein that is associated with gp41 by non-covalent interactions. In contrast, gp41 is an integral membrane protein, which is anchored in the membrane bilayer by a hydrophobic transmembrane domain located near the carboxyl terminus.

The amino acid sequence of gp120 contains five relatively conserved domains (C1-C5) interspersed with five hypervariable domains (V1-V5). The positions of 18 cysteine residues in the gp120 primary sequence, and the positions of 13 of the approximately 24 N-linked glycosylation sites in the gp120 sequence are conserved among most gp120 sequences. The hypervariable domains contain extensive amino acid substitutions, insertions and deletions. Sequence variations in these domains account for up to 30% overall sequence variability between gp120 molecules from the various viral isolates. Despite this variation, all gp120 sequences preserve the ability of the virus to interact with gp41 and to bind to the CD4 and chemokine (CCR5 and CXCR4) receptors to induce fusion of the viral and host cell membranes.

### SUMMARY OF THE INVENTION

The invention provides an immunogenic composition including an isolated polypeptide, or an isolated polynucleotide, and a pharmaceutically acceptable carrier. The isolated polypeptide includes, or the isolated polynucleotide encodes, a first gp120 amino acid sequence, wherein the first gp120 sequence includes at least the V2, V3, and C4 domains of gp120 and: (i) the first gp120 sequence lacks one or more cysteine residues at one or more of the following positions: 54, 74, 119, 126, 131, 157, 196, 205, 218, 228, 239, 247, 296, 331, 378, 385, 418, and 445; and/or (ii) the first gp120 sequence comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510, as numbered from the N-terminal methionine of gp120 from the HXB-2 strain of HIV gp120. However, the first gp120 sequence is not a subtype G gp120 sequence having one or more additional cysteines in the V1 domain or a subtype E gp120 sequence having one or more additional cysteines in the V4 domain. In preferred embodiments, the first gp120 sequence additionally comprises the V1 domain.

In one embodiment, the first gp120 sequence lacks one or more cysteine residues at one or more of the following positions: 54, 74, 119, 126, 157, 205, 218, 228, 239, 247, 331, 378, or 385.

In another embodiment, the first gp120 sequence includes one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120.

The first gp120 sequence can include a naturally occurring gp120 sequence and preferably includes a gp120 sequence from a primary isolate of HIV. In preferred embodiments, the first gp120 sequence has at least about 99% identity to each of the V1, V2, V3, and C4 domains of a gp120 selected from the group consisting of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136. In more preferred embodiments, the first gp120 sequence includes at least the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

In a preferred embodiment, the first gp120 sequence comprises an odd number of cysteines. In a variation of this embodiment, the immunogenic composition includes the polypeptide comprising the first gp120 sequence, and a cysteine in the first gp120 sequence is covalently bonded with another polypeptide, preferably via a disulfide bond. The other polypeptide can include a second gp120 sequence. Where the second gp120 sequence is the same as the first gp120 sequence, the first and second gp120 sequences form a homodimer. Where the second gp120 sequence is different from the first gp120 sequence, the first and second gp120 sequences form a heterodimer. The other polypeptide can additionally, or alternatively, include a gp41 amino acid sequence.

The invention also provides immunogenic compositions including the polypeptide comprising the first gp120 sequence, where a cysteine in the gp120 sequence is covalently bonded with an agent selected from the group consisting of a cell-specific binding moiety, a drug, an immunostimulatory oligonucleotide (e.g., CpG), and an immunogenic carrier protein.

In certain embodiments, the immunogenic composition includes a polypeptide including, or the polynucleotide encoding, a fusion polypeptide including the first gp120 sequence.

The immunogenic composition can additionally include an adjuvant, if desired.

The invention also provides an isolated polypeptide including a first gp120 amino acid sequence, wherein the first gp120 sequence comprises at least the V2, V3, and C4 domains of gp120 and: (a) the first gp120 sequence lacks one or more cysteine residues at one or more of the following positions: 54, 74, 119, 126, 157, 205, 218, 228, 239, 247, 331, 378, or 385; and/or (b) the first gp120 sequence comprises one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120, as numbered from the N-terminal methionine of gp120 from the HXB-2 strain of HIV gp120. However, the first gp120 sequence is not a subtype E gp120 sequence having one or more additional cysteines in the V4 domain. In preferred embodiments, the first gp120 sequence additionally comprises the V1 domain.

In one embodiment, the isolated polypeptide includes a first gp120 sequence lacking one or more cysteine residues at one or more of the following positions: 54, 74, 119, 126, 157, 205, 218, 228, 239, 247, 331, 378, or 385. In another embodiment, the isolated polypeptide includes a first gp120 sequence including one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120.

In preferred embodiments, the isolated polypeptide includes a first gp120 amino acid sequence that has at least about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136. In more preferred embodiments, the first gp120 sequence includes at least the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70,72, 74, 76, 78, 80, 82, 84, 86, 88,90,92,94,96,98, 100, 102, 104, 106, 108, 110, 112, 114, 116, 118, 120, 122, 124, 126, 128, 130, 132, 134, and 136.

In a preferred embodiment, the isolated polypeptide includes a first gp120 sequence having an odd number of cysteines. In a variation of this embodiment, a cysteine in the first gp120 sequence is covalently bonded with another polypeptide, preferably via a disulfide bond. The other polypeptide can include a second gp120 sequence. Where the second gp120 sequence is the same as the first gp120 sequence, the first and second gp120 sequences form a homodimer. Where the second gp120 sequence is different from the first gp120 sequence, the first and second gp120 sequences form a heterodimer. The other polypeptide can additionally, or alternatively, include a gp41 amino acid sequence.

The invention also provides isolated polypeptides where a cysteine in the gp120 sequence is covalently bonded with an agent selected from the group consisting of a cell-specific binding moiety, a drug, an immunostimulatory oligonucleotide, and an immunogenic carrier protein.

In certain embodiments, the isolated polypeptide includes a fusion polypeptide including the first gp120 sequence. The fusion polypeptide can include a heterologous signal sequence, such as, e.g., the herpes simplex virus glycoprotein D (gD-1) signal sequence or the human tissue plasminogen activator signal sequence. The fusion polypeptide can additionally, or alternatively, include an epitope tag.

Also provided by the invention is an isolated polynucleotide encoding any of the isolated polypeptides of the invention. If intended for use in expression of the encoded polypeptide, the polynucleotide is preferably codon-optimized for expression in a host cell of a particular species. In preferred embodiments, the polynucleotide encodes a gp120 sequence that has at least about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136. In more preferred embodiments, the polynucleotide includes a gp120 nucleotide sequence selected from the group consisting SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 or a subsequence thereof, wherein the subsequence encodes at least the V1, V2, V3, and C4 domains of gp120 .

Other aspects of the invention include a vector including any of the polynucleotides of the invention and a host cell including the vector. Preferred vectors of the invention include expression vectors and viral vectors. Preferred host cells include mammalian and bacterial cells.

The invention also provides a method of producing a polypeptide including a first gp120 amino acid sequence. In one embodiment, the method entails: (a) introducing a vector of the invention into a cell; and (b) expressing the polypeptide. The cell can be in vivo or in culture. If in culture, the method can additionally entail recovering the polypeptide from the culture. In another embodiment, the polypeptide is produced by: (a) culturing a host cell of the invention, wherein the host cell includes an expression vector, and the host cell is cultured under conditions suitable for expression of the polypeptide; and (b) recovering the polypeptide from the culture.

Another aspect of the invention is a method of immunizing an animal with a polypeptide including a first gp120 sequence. This method entails administering an immunogenic composition of the invention to the animal.

The invention also provides a diagnostic method that entails contacting a biological sample from a subject with: (a) an isolated polypeptide including a gp120 amino acid sequence; and (b) determining whether the sample contains antibodies that specifically bind to the isolated polypeptide. The gp120 sequence includes at least the V2, V3, and C4 domains of gp120 and: (i) lacks one or more cysteine residues at one or more of the following positions: 54, 74, 119, 126, 131, 157, 196, 205, 218, 228, 239, 247, 296, 331, 378, 385, 418, and 445; and/or (ii) includes one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510, as numbered from the N-terminal methionine of gp120 from the HXB-2 strain of HIV gp120. However, the gp120 sequence is not a subtype G gp120 sequence having one or more additional cysteines in the V1 domain or a subtype E gp120 sequence having one or more additional cysteines in the V4 domain. In preferred embodiments, the gp120 sequence additionally includes the V1 domain.

In an alternative embodiment, the invention provides a diagnostic method that entails assaying a biological sample from a subject to determine whether the sample includes a polypeptide including, or a polynucleotide encoding, a gp120 amino acid sequence having an unusual number of cysteines. More specifically, the sample is assayed for a gp120 amino acid sequence that: (a) lacks one or more cysteine residues at one or more of the following positions: 54, 74, 119, 126, 131, 157, 196, 205, 218, 228, 239, 247, 296, 331, 378, 385, 418, and 445; and/or (b) comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510; as numbered from the N-terminal methionine of gp120 from the HXB-2 strain of HIIV gp120. Preferably, the gp120 sequence is not a subtype G gp120 sequence having one or more additional cysteines in the V1 domain or a subtype E gp120 sequence having one or more additional cysteines in the V4 domain.

In one variation of this embodiment, the assay is an immunoassay that entails contacting the sample with an antibody that specifically binds the gp120 sequence under conditions suitable for binding.

In another variation of this embodiment, the assay is a polynucleotide-based assay in which sample polynucleotides are contacted with a nucleic acid molecule that hybridizes specifically to a nucleotide sequence encoding the gp120 sequence under conditions suitable for hybridization. In a preferred embodiment, the nucleic acid molecule can be one of a pair of amplification primers, in which case the assay is conducted by contacting sample polynucleotides with both amplification primers under conditions suitable for amplification, and then determining whether an amplification product is produced. In another preferred embodiment, the nucleic acid molecule is a nucleic acid probe affixed to a solid phase, and the assay is conducted by determining whether sample polynucleotides hybridize specifically to the nucleic acid probe.

### BRIEF DESCRIPTION OF THE DRAWINGS

The figures show schematic diagrams of the HIV-1 gp120 amino acid sequence. For all figures, the numbering for each amino acid residue position is relative to the HIV-1 strain HXB2. Lines indicate the actual amino acid residue that was mutated, with the amino acid residues labeled using the standard single-letter abbreviations. All mutations are designated in the following format to show a substitution of one residue for another: X->Y at position Z (of HXB2) where X is the replaced amino acid, and Y is the replacement (i.e., new) amino acid. The five variable and five conserved regions are indicated as V1-V5 and C1-C5, respectively.

FIG. 1. Positions 54 and 126 have substitutions that alter the Cys residues to Arg residues in sample U-101cl. The total cysteine complement is 16 residues.

FIG. 2. Positions 131 and 228 have substitutions that alter the Cys residues to Arg and Tyr, respectively, in sample U-178c13. The total cysteine complement is 16 residues.

FIG. 3 shows the HIV-1 samples that have a total cysteine complement of 17 residues. The sample names are indicated in parentheses after each mutation.

FIG. 4 shows the HIV-1 samples that have a total cysteine complement of 19 residues. The sample names are indicated in parentheses after each mutation.

FIG. 5A-X shows the HIV-1 samples that have a total cysteine complement of 20 residues. The sample names are indicated below each representation. In this composite figure, only the region that has additional Cys residues is shown. In panels A-S, the number of residues in the loop (excluding the Cys residues) is shown in the lower, right-hand portion of the panel. Panel T, showing U-374c1 and U-234c10, represent one set of potential secondary structures based on the additional cysteines. Panel U shows different conformations for U-374c1 and U-208_{—}4c1, based on distinct Cys pairings. Additional Cys residues in V2 (U-033c1) (panel V), V4 (U-210c2) (panel W), and V5 (U-062_{—}2c5) (panel X) are also depicted.

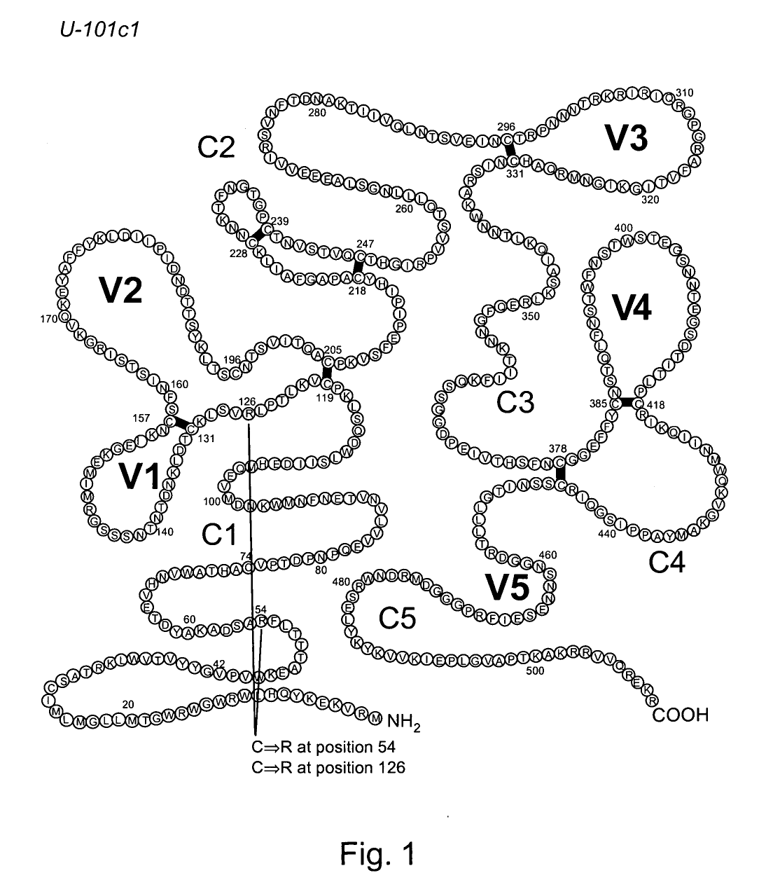

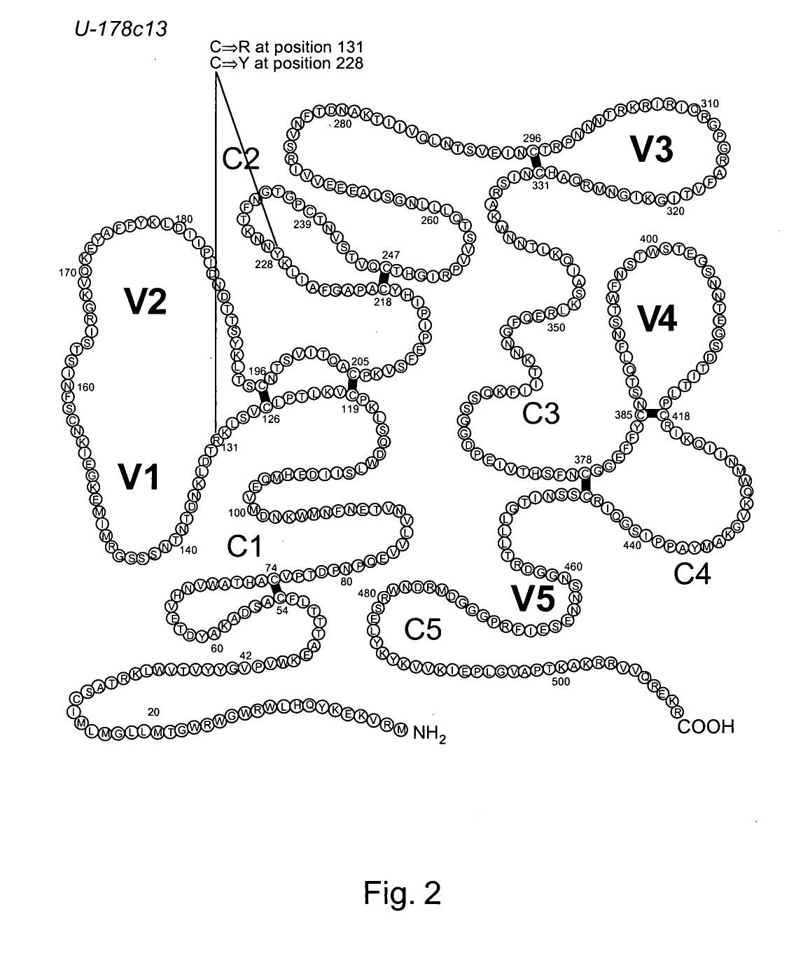

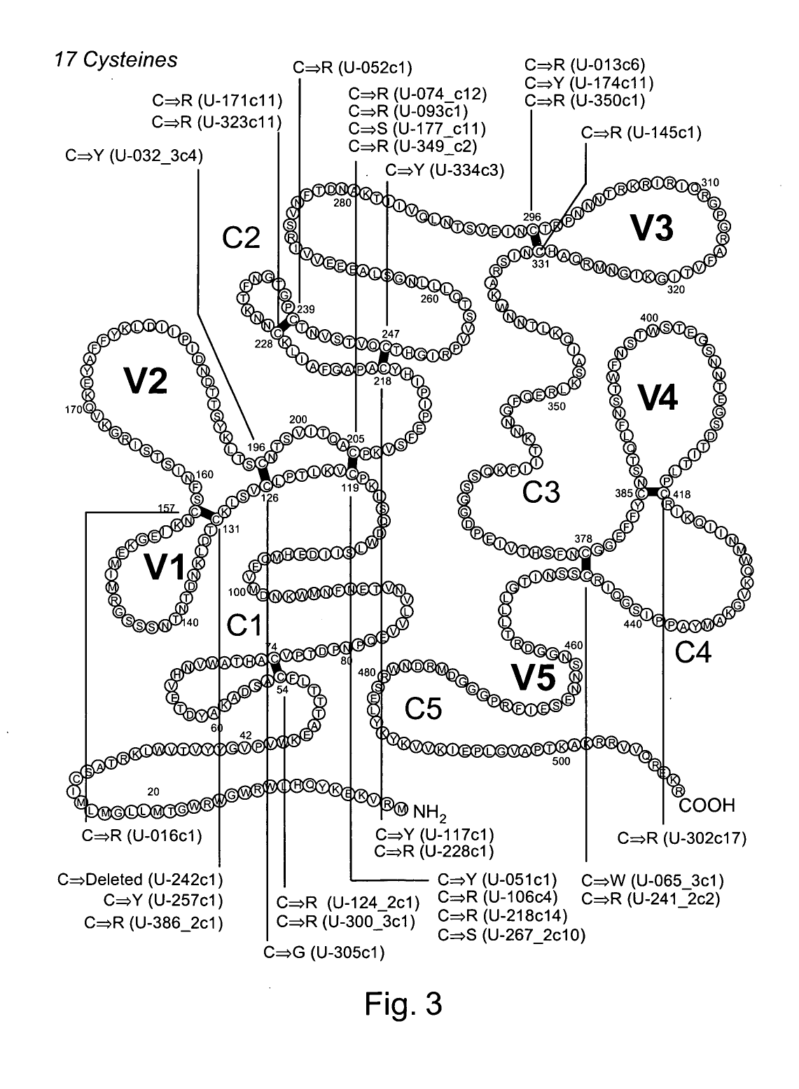

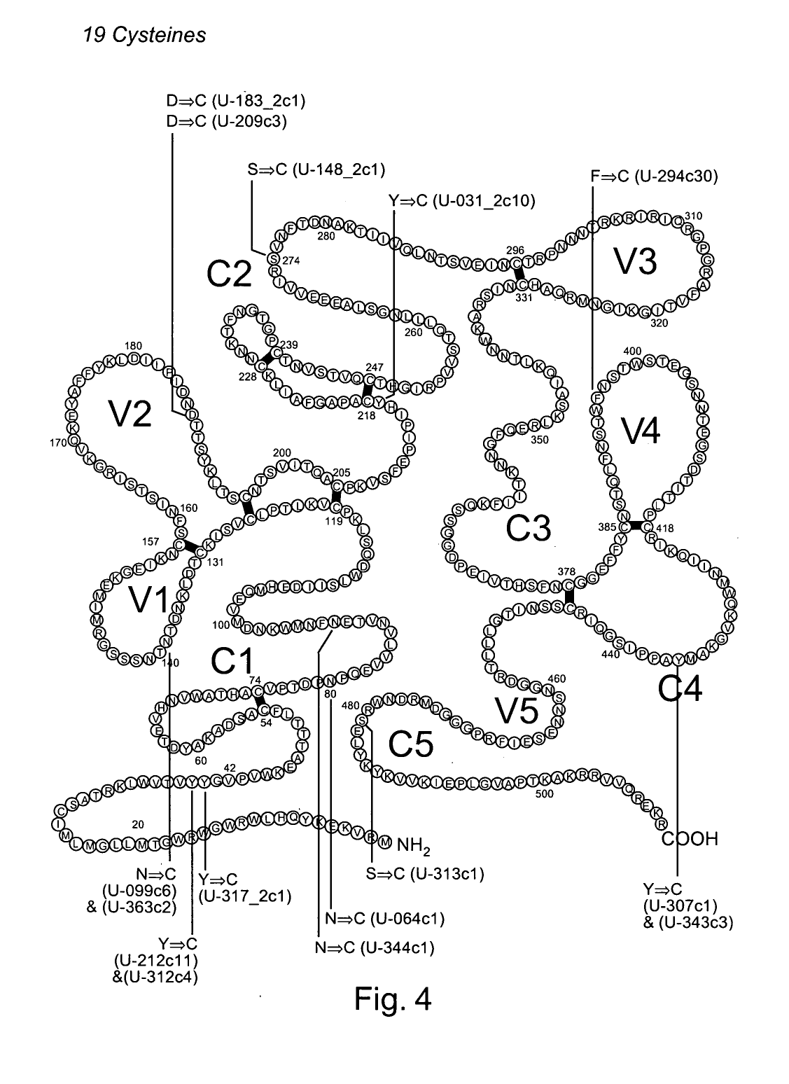

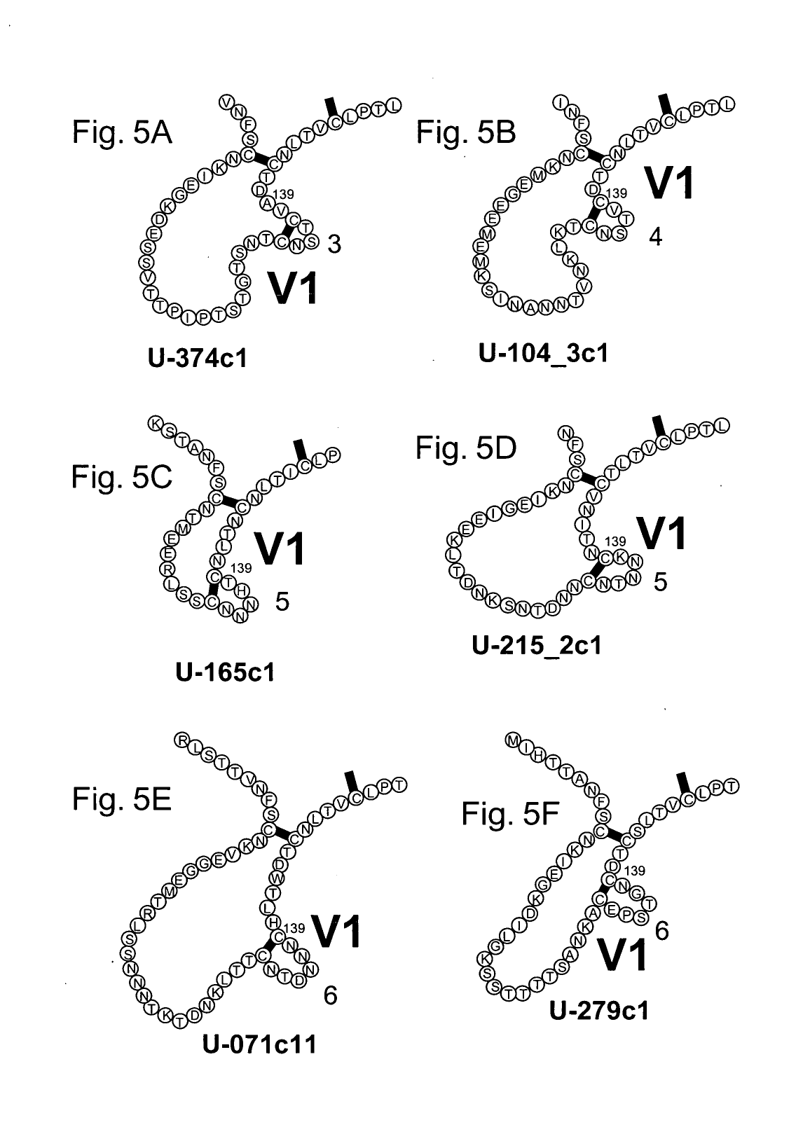

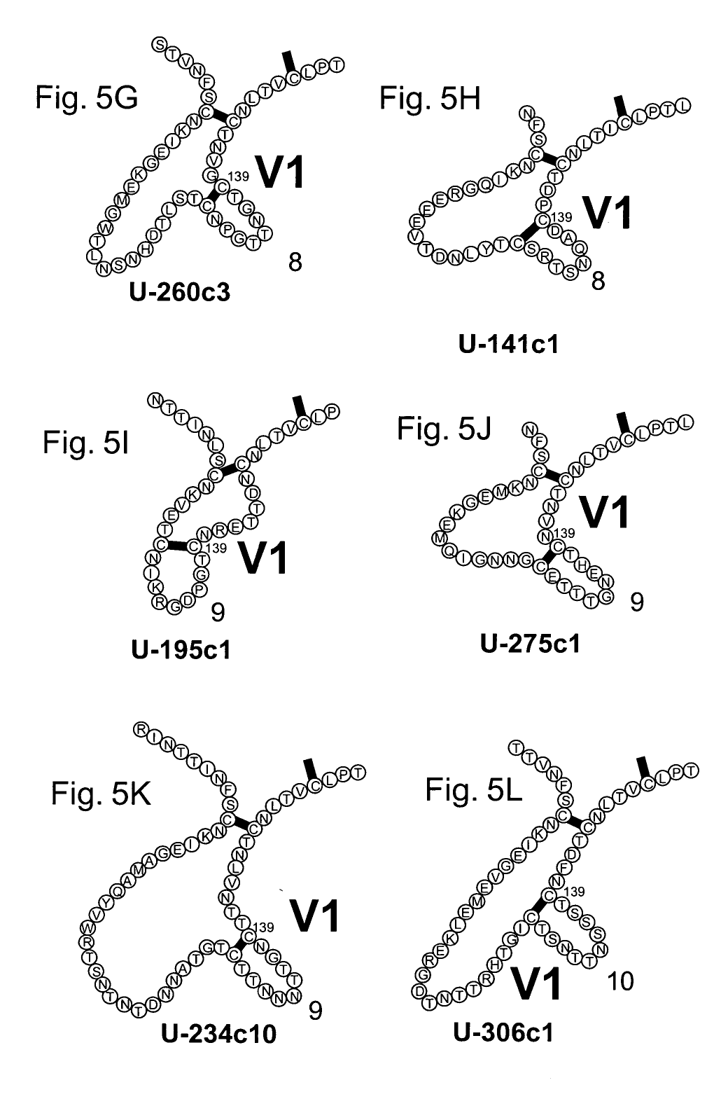

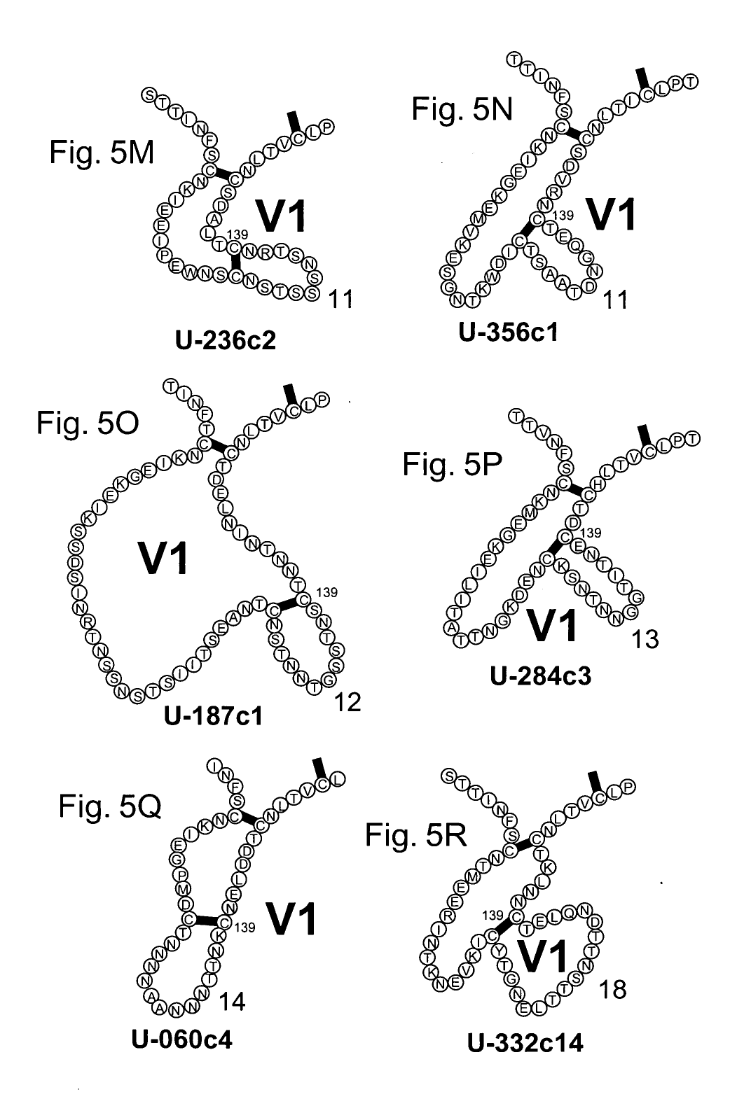

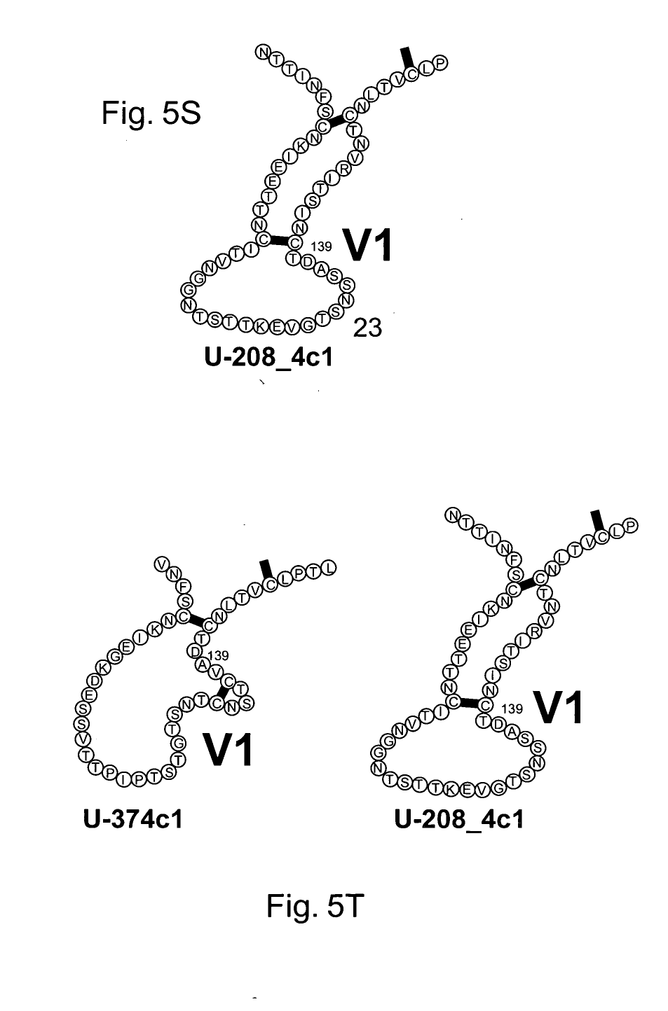

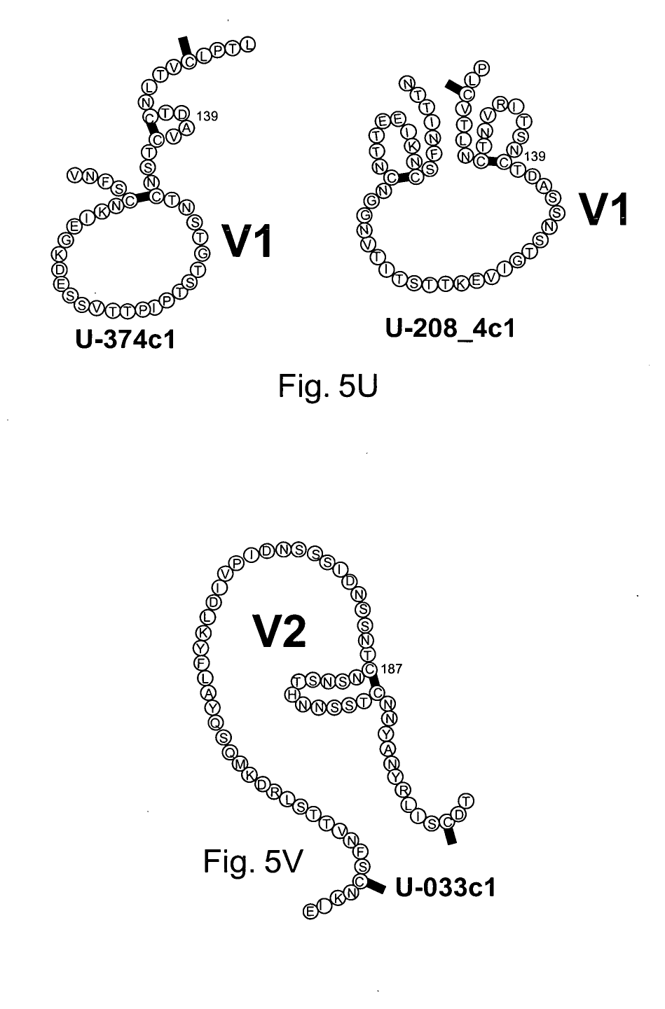

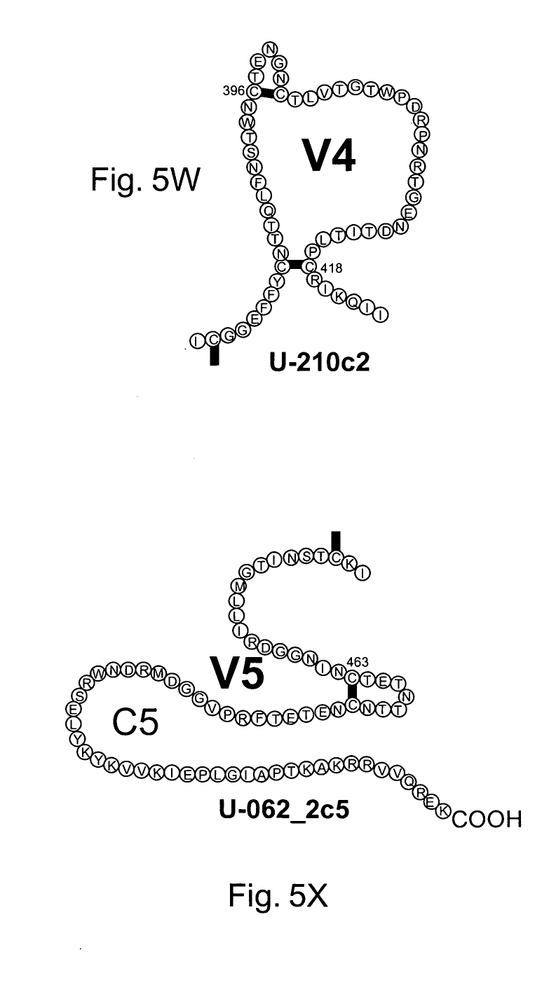

### DETAILED DESCRIPTION

The present invention provides isolated polypeptides that include sequences from HIV gp120. These gp120 sequences have unusual disulfide structure. Specifically, the gp120 sequences of the invention contain more or fewer than the usual 18 cysteine residues. The gp120 sequences described herein were obtained in a large-scale clinical trial of an HIV vaccine carried out at 61 clinical sites throughout North America, Puerto Rico, and the Netherlands. During the course of these studies, gp120 -encoding cDNAs from 350 recent HIV new infections were cloned and sequenced. In these studies, plasma samples were taken within 6 months of infection, and plasma viral RNA was reverse transcribed, amplified by the polymerase chain reaction (PCR), and sequenced. Surprisingly, approximately 20 percent of the gp120 sequences obtained had unusual disulfide structure. Thus, this structural feature is found in a significant number of successful viruses early after infection, before extensive mutation can occur in the host. Thus, viruses with unusual disulfide structure may represent a “transmission phenotype” associated with new infections or a major new variant of HIV-1 in circulation in North America. In either case, vaccines designed to prevent HIV-1 infection should be directed against viruses containing gp120s with unusual disulfide structure.

Naturally occurring proteins containing an odd number of cysteines are unusual because free sulfhydryl groups are highly reactive with other free sulfhydryl groups and therefore tend to form intramolecular and intermolecular disulfide bonds. gp120 sequences of the invention that contain an odd number of cysteines are of interest because the unpaired cysteine residue can form an extra inter-or intra- molecular disulfide bond and may represent a previously undescribed mechanism used by the virus to generate molecular diversity. In principle, such diversity could be generated by altering intra- or intermolecular disulfide structure, by mediating a covalvent linkage between two gp120 molecules (each with an unpaired cysteine residue), by mediating a covalent linkage with a cysteine reside in gp41, or by mediating a covalent linkage with a another viral or cellular protein.

Moreover, vaccine developers can make use of HIV-1 envelope proteins containing an unpaired cysteine residue in the creation of novel antigens that more accurately replicate the structure of gp120/gp41 complexes that occur on the surface of virions and virus-infected cells. For example, the viral spikes on the surface of the HIV-1 virions and virus-infected cells are thought to be non-covalent oligomers (usually trimers) of monomeric gp120/gp41 complexes. Because these complexes are fragile and disassociate, it has not been possible to purify monomeric or oligomeric gp120/gp41 complexes. It is believed, however, that superior HIV-1 vaccines could be developed if a method for producing oligomeric gp120/gp41 complexes could be developed. Indeed some investigators have used in vitro mutagenesis to engineer unpaired cysteine residues into the structures of gp120 and gp41 in order to create stable, covalently linked gp120/gp41 complexes. Although covalently linked complexes were achieved, the resulting structures were not naturally occurring structures and therefore did not accurately replicate the structure of the gp120/gp41 complexes, as they exist in virions or virus-infected cells. Because many of the molecules described in the present application are naturally occurring and have an unpaired cysteine in defined regions that are remote from those previously used for the production of disulfide stabilized gp120/gp41 complexes, these molecules offer a unique opportunity to genetically engineer a variety of disulfide-bonded structures to enhance the immunogenicity of gp120. Exemplary structures include homodimers, gp120/gp41 heterodimers, gp120s covalently linked to other HIV-1 proteins, gp120s linked to non-HIV-1 proteins, or gp120s covalently linked to non-protein chemical compositions. Such structures can enhance immunogenicity beyond that achievable with monomers. Heterodimeric gp120 complexes are useful to expand the breadth of the immune response. For example, a heterodimer containing gp120 sequences from two different HIV-1 subtypes can be employed to elicit an immune response against each HIV-1 subtype. In other embodiments, gp120 sequences containing a free sulfhydryl group can be linked to other moieties, for example, to target gp120 sequences to particular cell types (e.g., dendritic cells or macrophages) or to enhance immunogenicity (e.g., by linking gp120 sequences to a highly immunogenic carrier protein, such as diptheria toxin, keyhole limpet hemocyanin, thyroglobulin, or bovine serum albumin). Because gp120 binds with high affinity to the cell surface antigen CD4, gp120 sequences containing free sulfhydryl groups can be linked to a drug for targeting to CD4-bearing cells.

### Definitions

Terms used in the claims and specification are defined as set forth below unless otherwise specified.

A full-length gp120 amino acid sequence is said to have one or more “additional cysteines” if the sequence contains more than the 18 cysteine residues present in most full-length gp120 sequences. A gp120 fragment is also said to have “additional cysteines” if the full-length gp120 sequence from which it was derived contains more than the usual 18 cysteine residues, and one or more of the extra cysteine(s) is/are present in the fragment.

The term “immunogenic composition” refers to any composition that is capable of eliciting an immune response.

As used herein, the term “vaccine” refers to an immunogenic composition that reduces the risk of, or prevents, infection by an infectious agent (a “prophylactic vaccine”) or that ameliorates, to any extent, an existing infection (a “therapeutic vaccine”). If a vaccine protects an organism from subsequent challenge with the infectious agent, the vaccines is said to be “protective.”

As used herein, the term “DNA vaccine” is a vaccine containing one or more polynucleotides encoding an antigen, wherein administration of the polynucleotide to an organism results in expression of the encoded antigen, followed by an immune response to that antigen.

As used herein, the term “virus-derived vaccine” refers to a vaccine containing a viral particle, a virus-like particle (VLP), some portion of a viral particle or VLP, and/or a virally infected cell that displays the antigen on its surface, wherein administration of the particle or cell to an organism elicits an immune response to the displayed antigen. The term “virus-derived vaccine” encompasses chimeric viral particles or VLPs, which contain components from two or more different sources.

The terms “polypeptide” and “protein” are used interchangeably herein to refer to a polymer of amino acids, and unless otherwise limited, include atypical amino acids that can function in a similar manner to naturally occurring amino acids.

As used with respect to polypeptides or polynucleotides, the term “isolated” refers to a polypeptide or polynucleotide that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad), which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2, 3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

As used with reference to a polypeptide, the term “full-length” refers to a polypeptide having the same length as the mature wild-type polypeptide.

The term “fragment” is used herein with reference to a polypeptide or a polynucleotide to describe a portion of a larger molecule. Thus, a polypeptide fragment can lack an N-terminal portion of the larger molecule, a C-terminal portion, or both. Polypeptide fragments are also referred to herein as “peptides.” A fragment of a polynucleotide can lack a 5′ portion of the larger molecule, a 3′ portion, or both. Polynucleotide fragments are also referred to herein as “oligonucleotides.” Oligonucleotides are relatively short nucleic acid molecules, generally shorter than 200 nucleotides, more particularly, shorter than 100 nucleotides, most particularly, shorter than 50 nucleotides. Typically, oligonucleotides are single-stranded DNA molecules.

A “subsequence” of an amino acid or nucleotide sequence is a portion of a larger sequence.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm (Basic Local Alignment Search Tool), which is described in Altschul et al. (1990) J. Mol. Biol. 215: 403-410. Software for performing BLAST analyses is publicly available through the National Center for Biotechnology Information (http:Hlwww.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA ,90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Residues in two or more polypeptides are said to “correspond” if they are either homologous (i.e., occupying similar positions in either primary, secondary, or tertiary structure) or analogous (i.e., having the same or similar functional capacities). As is well known in the art, homologous residues can be determined by aligning the polypeptide sequences for maximum correspondence as described above.

Positions in gp120 amino acid sequences described in the application are identified by position numbers, wherein the numbering is from the N-terminal methionine of gp120 from the HXB-2 strain of HIV (SEQ ID NO:138).

A gp120 amino acid sequence is said to lack a cysteine at a particular position if another amino acid residue is substituted for the cysteine that is usually present at that position or if the cysteine has been deleted.

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed using recombinant technology, followed by purification; or synthesized.

The term “amino acid sequence variant” refers to a polypeptide having an amino acid sequence that differs from a wild-type amino acid sequence by the addition, deletion, or substitution of an amino acid.

The term “conservative amino acid substitution” is used herein to refer to the replacement of an amino acid with a functionally equivalent amino acid. Functionally equivalent amino acids are generally similar in size and/or character (e.g., charge or hydrophobicity) to the amino acids they replace. Amino acids of similar character can be grouped as follows:

(1) hydrophobic: His, Trp, Tyr, Phe, Met, Leu, Ile, Val, Ala;

(2) neutral hydrophobic: Cys, Ser, Thr;

(3) polar: Ser, Thr, Asn, Gln;

(4) acidic/negatively charged: Asp, Glu;

(5) charged: Asp, Glu, Arg, Lys, His;

(6) basic/positively charged: Arg, Lys, His;

(7) basic: Asn, Gln, His, Lys, Arg;

(8) residues that influence chain orientation: Gly, Pro; and

(9) aromatic: Trp, Tyr, Phe, His.

The following table shows exemplary and preferred conservative amino acid substitutions.

| Original | | Preferred Conservative |
|---|---|---|
| Residue | Exemplary Conservative Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Asn |
| Ile | Leu, Val, Met, Ala, Phe | Leu |
| Leu | Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala | Leu |

A “signal sequence” is an amino acid sequence that is found on membrane-bound bound and secreted proteins that directs the synthesis of proteins to membrane-bound ribosomes and mediates translocation of nascent peptide chains into the lumen of the endoplasmic reticulum, where a variety of post translational modifications (e.g., glycosylation) are available that are not available to proteins synthesized in the cytoplasm on free ribosomes. As used in recombinant expression, the polypeptide is secreted from a cell expressing the polypeptide into the culture medium for ease of purification. A signal sequence is said to be “heterologous” if the signal sequence is derived from a polypeptide other than the polypeptide to which it is fused to facilitate recombinant expression or secretion.

An “epitope tag” is an amino acid sequence that defines an epitope for an antibody. Epitope tags can be engineered into polypeptides or peptides of interest to facilitate purification or detection.

As used herein, a “fusion polypeptide” is a polypeptide that includes an amino acid sequence from one polypeptide linked to an amino acid sequence not normally present in that polypeptide. The latter may be an amino acid sequence from a different polypeptide (e.g., a heterologous signal sequence) or an artificial sequence. Generally, fusion polypeptides are expressed using recombinant technology that utilizes a construct containing a nucleotide sequence encoding one polypeptide sequence fused, in frame, to a nucleotide sequence encoding the other polypeptide sequence.

As used with reference to a polypeptide or polypeptide fragment, the term “derivative” includes amino acid sequence variants as well as any other molecule that differs from a wild-type amino acid sequence by the addition, deletion, or substitution of one or more chemical groups. “Derivatives” retain at least one biological or immunological property of a wild-type polypeptide or polypeptide fragment, such as, for example, the biological property of specific binding to a receptor and the immunological property of specific binding to an antibody.

A “cell-specific binding moiety” refers to a moiety that binds specifically to a binding partner found on one or several particular cell types. A cell-specific binding moiety can be linked to a polypeptide or polynucleotide, for example, to direct the polypeptide or polynucleotide to a desired cell type.

An “immunogenic carrier protein” is a polypeptide that is linked to an antigen that does not, by itself, elicit a significant immune response (i.e., a “hapten”). The resulting conjugate is capable of eliciting an immune response against the hapten.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50 -70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab, which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer, which may be expressed from a nucleic acid including VH- and VL- encoding sequences either joined directly or joined by a peptide-encoding linker. Huston, et al. (1988) Proc. Nat. Acad. Sci. USA, 85: 5879-5883. While the VH and VL are connected to form a single polypeptide chain, the VH and VL domains associate non-covalently. The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778).

The phrases “an effective amount” and “an amount sufficient to” refer to amounts of a biologically active agent that produce an intended biological activity.

The term “pharmaceutically acceptable” refers to any agent that is sufficiently non-toxic to cells and/or subjects to allow pharmaceutical use of the agent.

The term “adjuvant” refers to a compound or mixture that enhances the immune response to an antigen.

A “primary isolate” of HIV is an HIV isolate obtained from an individual infected with HIV without passaging in cell culture.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers to any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and MRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

The term “vector” is used herein to describe a DNA construct containing a polynucleotide. Preferred vectors can be propagated stably or transiently in a host cell. The vector can, for example, be a plasmid, cosmid, bacterial artificial chromosome (BAC), yeast artificial chromosome (YAC), or a viral vector. Once introduced into a suitable host, the vector may replicate and function independently of the host genome, or may, in some instances, integrate into the host genome.

As used herein, the term “operably linked” refers to a functional linkage between a control sequence (typically a promoter) and the linked sequence. For example, a promoter is operably linked to a sequence if the promoter can initiate transcription of the linked sequence.

“Expression vector” refers to a DNA construct containing a polynucleotide that is operably linked to a control sequence capable of effecting the expression of the polynucleotide in a suitable host. Exemplary control sequences include a promoter to effect transcription, an optional operator sequence to control transcription, a sequence encoding suitable mRNA ribosome binding sites, and sequences that control termination of transcription and translation.

The term “host cell” refers to a cell capable of maintaining a vector either transiently or stably. Host cells of the invention include, but are not limited to, bacterial cells, yeast cells, insect cells, plant cells and mammalian cells. Other host cells known in the art, or which become known, are also suitable for use in the invention.

A host cell may be present in a cell culture or, alternatively, “in vivo.” A host cell is “in vivo” when it is present in a living organism.

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH 7.0.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

### Isolated gp120 Polypeptides Having Unusual Disulfide Structure

The present invention provides an isolated polypeptide that includes a first gp120 amino acid sequence. The isolated polypeptide can include one or more additional sequences, and thus the isolated polypeptide can include a full-length gp160 sequence or a gp120 sequence-containing fragment of gp160. The first gp120 sequence can be a full-length gp120 sequence or a fragment thereof. A gp120 fragment useful in the invention includes at least the V2, V3, and C4 domains of gp120. In preferred embodiments, the gp120 fragment also includes the V1 domain. The gp120 sequence lacks one or more of the 18 cysteine residues present in the majority of gp120 sequences or includes one or more additional cysteines.

gp120 polypeptides of the invention can be used as components of the immunogenic compositions of the invention to elicit a gp120-specific immune response. In preferred embodiments, the gp120 polypeptides are used as vaccine antigens. The polypeptides can be included in a vaccine in a variety of forms, e.g., in free form, covalently bonded to a cell-specific moiety or an immunogenic carrier protein, or displayed on the surface of a viral particle (as in a virus-derived vaccine). In addition, gp120 polypeptides that carry a drug can be used to target the drug to CD4- or chemokine receptor-bearing cells.

The gp120 polypeptides of the invention are also useful in a diagnostic method to determine whether a sample contains an antibody specific for a given gp120 polypeptide with an unusual disulfide structure.

Furthermore, suitable polypeptides of the invention can be used as standards in gp120 immunoassays. More specifically, any polypeptide of the invention that cross-reacts with the anti-HIV-1 antibody employed in the assay can be used as a positive control, which can be compared with the results observed when a sample is assayed for the presence of gp120.

A. Types of gp120 Polypeptides

In a first embodiment, the first gp120 sequence lacks one or more cysteine residues at one or more of the following positions: 54, 74, 119, 126, 131, 157, 196, 205, 218, 228, 239, 247, 296, 331, 378, 385, 418, and 445, as numbered from the N-terminal methionine of gp120 from the HXB-2 strain of HIV gp120. In a preferred variation of this embodiment, the first gp120 sequence lacks one or more cysteine residues at one or more of the following positions: 54, 74, 119, 126, 157, 205, 218, 228, 239, 247, 331, 378, and 385.

In a second embodiment, the first gp120 sequence includes one or more additional cysteine residues at a position other than the following positions: 24, 29, 34, 54, 74, 119, 126, 131, 157, 196, 205,218, 228, 239, 247, 296,331, 378, 385, 418, 445, 493, 495, 499-501, 503-508, and 510, as numbered based on HXB-2 gp120. However, the first gp120 sequence of the invention is not a subtype G gp120 sequence having one or more additional cysteines in the V1 domain, nor is it a subtype E gp120 sequence having one or more additional cysteines in the V4 domain. In a preferred variation of this embodiment, the first gp120 sequence comprises one or more additional cysteine residues at a position other than the following positions: 54, 74, 119, 126, 131, 157, 196, 205, 218, 228, 239, 247, 296, 331, 378, 385, 418, and 445, provided that the one or more additional cysteine residues are not present in the V1 domain of gp120.

The invention also includes combinations of these two embodiments, i.e., where the first gp120 sequence lacks one or more cysteines at a position(s) noted above for the first embodiment and includes one or more additional cysteines at a position(s) noted above for the second embodiment.

In preferred embodiments, the first gp120 sequence has at least about 80%, about 85%, about 90%, about 95%, or about 99% identity to each of the V2, V3, and C4 domains of a gp120 sequence selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136. These gp120 sequences were obtained in a large-scale clinical trial of an HIV vaccine carried out at 61 clinical sites throughout North America, Puerto Rico, and the Netherlands. Table 1, below, shows the correspondence between the SEQ ID NO and the gp120 sample designation from the trial. The amino acid sequence for gp120 from the HXB-2 strain of HIV-1 is SEQ ID NO:138.

In more preferred embodiments, the first gp120 sequence has at least about 80%, about 85%, about 90%, about 95%, or about 99% identity to each of the V1, V2, V3, and C4 domains of a gp120 sequence selected from one of these sequences. This requirement is met when percent identity for each one of these domains is at least about 80%, about 85%, about 90%, about 95%, or about 99% or greater. The endpoints of these domains, as numbered from the N-terminal methionine of HXB-2 are as follows: V1: residues 131 to 157; V2: residues 157 to 196; V3: residues 296 to 331; C4: residues 418 to 445. In particular examples of such embodiments, the first gp120 sequence includes at least the V1, V2, V3, and C4 domains of a gp120 sequence selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

TABLE 1
Sequence ID Numbers for HIV-1 gp120
DNA and Protein Sequences.

| SEQ ID NO | HIV-1 gp120 Sample/Sequence Type |
|---|---|
| 1 | U-101c1/DNA |
| 2 | U-101c1/protein |
| 3 | U-178c13/DNA |
| 4 | U-178c13/protein |
| 5 | U-013c6/DNA |
| 6 | U-013c6/protein |
| 7 | U-016c1/DNA |
| 8 | U-016c1/protein |
| 9 | U-032_3c4/DNA |
| 10 | U-032_3c4/protein |
| 11 | U-051c1/DNA |
| 12 | U-051c1/protein |
| 13 | U-052c1/DNA |
| 14 | U-052c1/protein |
| 15 | U-065_3c1/DNA |
| 16 | U-065_3c1/protein |
| 17 | U-074c12/DNA |
| 18 | U-074c12/protein |
| 19 | U-093c1/DNA |
| 20 | U-093c1/protein |
| 21 | U-106c4/DNA |
| 22 | U-106c4/protein |
| 23 | U-117c1/DNA |
| 24 | U-117c1/protein |
| 25 | U-124_2c1/DNA |
| 26 | U-124_2c1/protein |
| 27 | U-145c1/DNA |
| 28 | U-145c1/protein |
| 29 | U-171c11/DNA |
| 30 | U-171c11/protein |
| 31 | U-174c11/DNA |
| 32 | U-174c11/protein |
| 33 | U-177c11/DNA |
| 34 | U-177c11/protein |
| 35 | U-218c14/DNA |
| 36 | U-218c14/protein |
| 37 | U-228c1/DNA |
| 38 | U-228c1/protein |
| 39 | U-241_2c2/DNA |
| 40 | U-241_2c2/protein |
| 41 | U-242c1/DNA |
| 42 | U-242c1/protein |
| 43 | U-257c1/DNA |
| 44 | U-257c1/protein |
| 45 | U-267_2c10/DNA |
| 46 | U-267_2c10/protein |
| 47 | U-300_3c1/DNA |
| 48 | U-300_3c1/protein |
| 49 | U-302c17/DNA |
| 50 | U-302c17/protein |
| 51 | U-305c1/DNA |
| 52 | U-305c1/protein |
| 53 | U-323c11/DNA |
| 54 | U-323c11/protein |
| 55 | U-334c3/DNA |
| 56 | U-334c3/protein |
| 57 | U-349c2/DNA |
| 58 | U-349c2/protein |
| 59 | U-350c1/DNA |
| 60 | U-350c1/protein |
| 61 | U-386_2c1/DNA |
| 62 | U-386_2c1/protein |
| 63 | U-031_2c10/DNA |
| 64 | U-031_2c10/protein |
| 65 | U-064c1/DNA |
| 66 | U-064c1/protein |
| 67 | U-099c6/DNA |
| 68 | U-066c6/protein |
| 69 | U-148_2c1/DNA |
| 70 | U-148_2c1/protein |
| 71 | U-183_2c1/DNA |
| 72 | U-183_2c1/protein |
| 73 | U-209c3/DNA |
| 74 | U-209c3/protein |
| 75 | U-212c11/DNA |
| 76 | U-212c11protein |
| 77 | U-294c30/DNA |
| 78 | U-294c30/protein |
| 79 | U-307c1/DNA |
| 80 | U-307c1/protein |
| 81 | U-312c4/DNA |
| 82 | U-312c4/protein |
| 83 | U-313c1/DNA |
| 84 | U-313c1/protein |
| 85 | U-317_2c1/DNA |
| 86 | U-317_2c1/protein |
| 87 | U-343c3/DNA |
| 88 | U-343c3/protein |
| 89 | U-344c1/DNA |
| 90 | U-344c1/protein |
| 91 | U-363c2/DNA |
| 92 | U-363c2/protein |
| 93 | U-033c1/DNA |
| 94 | U-033c1/protein |
| 95 | U-060c4/DNA |
| 96 | U-060c4/protein |
| 97 | U-062_2c5/DNA |
| 98 | U-062_2c5/protein |
| 99 | U-071c11/DNA |
| 100 | U-071c11/protein |
| 101 | U-104_3c1/DNA |
| 102 | U-104_3c1/protein |
| 103 | U-141c1/DNA |
| 104 | U-141c1/protein |
| 105 | U-165c1/DNA |
| 106 | U-165c1/protein |
| 107 | U-187c1/DNA |
| 108 | U-187c1/protein |
| 109 | U-195c1/DNA |
| 110 | U-195c1/protein |
| 111 | U-208_4c1/DNA |
| 112 | U-208_4c1/protein |
| 113 | U-210c2/DNA |
| 114 | U-210c2/protein |
| 115 | U-215_2c1/DNA |
| 116 | U-215_2c1/protein |
| 117 | U-234c10/DNA |
| 118 | U-234c10/protein |
| 119 | U-236c2/DNA |
| 120 | U-236c2/protein |
| 121 | U-260c3/DNA |
| 122 | U-260c3/protein |
| 123 | U-275c1/DNA |
| 124 | U-275c1/protein |
| 125 | U-279c1/DNA |
| 126 | U-279c1/protein |
| 127 | U-284c3/DNA |
| 128 | U-284c3/protein |
| 129 | U-306c1/DNA |
| 130 | U-306c1/protein |
| 131 | U-332c14/DNA |
| 132 | U332c14/protein |
| 133 | U-356c1/DNA |
| 134 | U-356c1/protein |
| 135 | U-374c1/DNA |
| 136 | U-374c1/protein |
| 137 | HXB-2/DNA |
| 138 | HXB-2/protein |

The gp120 amino acid sequence can be a naturally occurring (i.e., wild-type) amino acid sequence or an amino acid sequence variant of the corresponding region of a wild-type polypeptide. In one embodiment, the gp120 amino acid sequence is from a primary isolate of HIV-1. Preferred polypeptides of the invention generally include a wild-type gp120 amino acid sequence or a gp120 amino acid sequence containing conservative amino acid substitutions, as defined above.

Polypeptides of the invention can include other amino acid sequences, in addition to gp120 amino acid sequences, i.e., amino acid sequences from heterologous proteins. Accordingly, the invention encompasses fusion polypeptides in which the gp120 amino acid sequence is fused, at either or both ends, to amino acid sequence(s) from one or more heterologous proteins. Examples of additional amino acid sequences often incorporated into proteins of interest include a signal sequence, which facilitates secretion of the protein, and an epitope tag, which can be used for immunological detection or affinity purification. Preferred signal sequences for use in the invention include, but are not limited to, the herpes simplex virus glycoprotein D (HSV gD-1) signal sequence and the human tissue plasminogen activator signal sequence. Exemplary epitope tags include green fluorescent protein, hemagglutinin, or FLAG epitope tags and hexahistidine or similar metal affinity tags. An N-terminal HSV gD-1 sequence is conveniently employed as an epitope tag when the HSV gD-1 signal sequence is used to facilitate secretion.

Polypeptides of the invention can be otherwise modified to produce derivatives that retain the ability to elicit a gp120-specific immune response. For example, those of skill in the art recognize that a variety of techniques are available for constructing so-called “peptide mimetics” with the same or similar desired biological activity as the corresponding peptide compound, but with more favorable activity than the peptide with respect to, e.g., solubility, stability, and susceptibility to hydrolysis and proteolysis. See, for example, Morgan, et al., Ann. Rep. Med. Chem., 24:243-252 (1989). Accordingly, the polypeptides of the invention include peptide mimetics that are, for example, modified at the N-terminal amino group, the C-terminal carboxyl group, and/or wherein one or more of the amido linkages in the peptide is/are converted to a non-amido linkage.

In particular embodiments, polypeptides of the invention include a first gp120 sequence that has an odd number of cysteine residues. Such a sequence has at least one “free” cysteine (i.e., one that does not form an intramolecular disulfide bond). The free cysteine can be covalently bonded with another polypeptide. For example, the free cysteine can form a disulfide bond with a free cysteine in another polypeptide. A free cysteine can conveniently be inserted into any polypeptide of interest using site-directed mutagenesis, for example. The other polypeptide can include a second gp120 sequence, which can be the same as, or different from, the first gp120 sequence. Disulfide bonding between two identical gp120 sequences produces a homodimer. Disulfide bonding between two different gp120 sequences produces a heterodimer.

In a variation of this embodiment, a free cysteine in the first gp120 sequence is covalently bonded to another polypeptide that comprises a gp41 amino acid sequence. Preferably, the first gp120 sequence is disulfide bonded to a free cysteine in the gp41 sequence to produce a gp120/gp41 complex. Such complexes are useful as antigens because they can mimic the gp120/gp41 complexes found in viral spikes on the surface of HIV-1 viral particles or virus-infected cells.

In other embodiments, a free cysteine in the gp120 sequence is covalently bonded with a cell-specific binding moiety, a drug, an immunostimulatory oligonucleotide (e.g., CpG), or an immunogenic carrier protein. Targeting derivatized gp120s to antigen-presenting cells (such as dendritic cells or macrophages) in this way can be useful in modulating the potency and quality of the immune response (e.g., TH1 or TH2 immune responses). Cell-specific binding moieties useful in the invention include any moiety (e.g., a ligand or fragment thereof) capable of binding to a specific ligand-binding sites located on the target cell or cells (e.g., leukocytes) and not found in significant amounts on non-target cells (e.g., liver or muscle cells). Generally, cell-specific binding moieties bind to a membrane-bound cell-surface protein, carbohydrate, lipid, glycosaminoglycan, lipoprotein, antigen, or receptor. Thus, exemplary cell-specific binding moieties can include, for example, ligands, such as hormones or cytokines, receptor-binding domains of hormones or cytokines, adhesion molecules, and antibodies. Commonly used immunogenic carriers suitable for use in the invention include diptheria toxin, keyhole limpet hemocyanin (KLH), thyroglobulin, bovine serum albumin (BSA), and tetanus toxoid.

B. Production of gp120 Polypeptides

1. Synthetic Techniques

Polypeptides according to the invention can be synthesized using methods known in the art, such as for example exclusive solid phase synthesis, partial solid phase synthesis, fragment condensation, and classical solution synthesis. See, e.g., Merrifield, J. Am. Chem. Soc., 85:2149 (1963). Solid phase techniques are preferred and are described, for example, in John Morrow Stewart and Janis Dillaha Young, Solid Phase Peptide Syntheses (2nd Ed., Pierce Chemical Company, 1984). On a solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. Automated peptide synthesizers are commercially available, as are services that make peptides to order.

2. Recombinant Techniques

Polypeptides according to the invention can also produced using recombinant techniques. gp120 polynucleotides can be produced synthetically, amplified (by PCR, RT-PCR, rolling circle amplification (RCA) or other amplification method) from cDNA reverse-transcribed from viral RNA, proviral DNA, or a cloned gp120 polynucleotide or HIV-1 isolate, or otherwise cloned from an HIV-1 isolate. With a given gp120 polynucleotide in hand, a polynucleotide encoding a desired gp120 amino acid sequence can be generated by any of a variety of cloning and mutagenesis techniques. See, e.g., Sambrook, J., Fritsch, E. F., and Maniatis, T. (1989) in Molecular Cloning: A Laboratory Manual, Second Edition, Cold Spring Harbor, N.Y. Examples of widely used mutagenesis techniques include site-directed mutagenesis (Kunkel et al., (1991) Methods Enzymol., 204:125-139; Carter, P., et al., (1986) Nucl. Acids Res. 10:6487), cassette mutagenesis (Wells, J. A., et al., (1985) Gene 34:315), and restriction selection mutagenesis (Wells, J. A., et al., (1986) Philos. Trans. R. Soc., London Ser. A, 317:415).

In a preferred embodiment of the invention, the sequence of a gp120 coding region is used as a guide to design a synthetic polynucleotide encoding a desired gp120 sequence that can be incorporated into a vector of the present invention. Methods for constructing synthetic genes are well known to those of skill in the art. See, e.g., Dennis, M. S., Carter, P. and Lazarus, R. A. (1993) Proteins: Struct. Funct. Genet., 15:312-321. Expression and purification methods are described in greater detail below in connection with the nucleic acids, vectors and host cells of the invention.

3. Complexes and Conjugates

gp120 polypeptides of the invention can be modified by techniques commonly used for producing polypeptide complexes and conjugates. Disulfide-bonded oligomeric complexes including one or more gp120 polypeptides can be produced, for example, by mixing the polypeptides to be complexed in a solution containing a mild reducing agent, such as glutathione, dithiothreitol (DTT), or β-mercaptoethanol, and, optionally, a denaturant, such as urea or guanidine hydrochloride. The reducing agent and optional denaturant can be removed by dialysis. Disulfide bonds form during renaturation in the presence of air, which oxidizes the reduced sulfhydryl groups. Intramolecular disulfide bonds produce oligomers. If the solution contains only one type of gp120 polypeptide, the oligomers formed will contain that species. If the solution contains different gp120 polypeptides or a gp120 polypeptide in combination with another polypeptide (e.g., gp41), the oligomers formed can contain multiple species.

Alternatively, oligomeric complexes including one or more gp120 polypeptides of the invention can be produced using, e.g., standard bifunctional cross-linking agents. Such agents can also be employed to produce conjugates of gp120 polypeptides with a cell-specific binding moiety, a drug, an immunostimulatory oligonucleotide, or an immunogenic carrier protein.

For some applications, such as the diagnostic methods described below, it is desirable to attach a detectable label to one or gp120 polypeptides of the invention. Detectable labels suitable for use in the present invention include any composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oregon, USA), radiolabels (e.g., ³H, ¹²⁵I, ³⁵S, ¹⁴C, or ³²p), enzymes (e.g., horse radish peroxidase, alkaline phosphatase and others commonly used in an ELISA), and colorimetric labels such as colloidal gold (e.g., gold particles in the 40−80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

### Isolated Polvnucleotides Encoding gp120 Polypeptides Having Unusual Disulfide Structure, Vectors, and Host Cells

The invention also provides an isolated polynucleotide that encodes a polypeptide of the invention. Accordingly, polynucleotides of the invention include a DNA or RNA portion that encodes a gp120 amino acid sequence. The polynucleotides of the invention are useful for recombinant production of the polypeptides of the invention in vivo (i.e., in an organism) or in vitro (e.g., in cell culture). In particular embodiments, described further below, the polynucleotides of the invention can be used in immunogenic compositions, such as DNA vaccines, recombinant viruses, or virus-derived vaccines (i.e., as components of viral particles). In addition, the gp120 polynucleotides of the invention can be used in a diagnostic method to determine whether a sample contains a polynucleotide that encodes a gp120 polypeptide with an unusual disulfide structure.

A. Polynucleotides

In certain embodiments, the polynucleotide encodes a gp120 sequence that has at least about 80%, about 85%, about 90%, about 95%, or about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136. In preferred embodiments, the polynucleotide includes a gp120 nucleotide sequence selected from the group consisting SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77,79, 81, 83, 85, 87, 89, 91, 93, 95, 97, 99, 101, 103, 105, 107, 109, 111, 113, 115, 117, 119, 121, 123, 125, 127, 129, 131, 133, 135 or a subsequence thereof, wherein the subsequence encodes at least the V1, V2, V3, and C4 domains of gp120. Table 1, above, shows the correspondence between the SEQ ID NO and the gp120 sample designation from the clinical trial discussed above. The nucleotide sequence for gp120 from the HXB-2 strain of HIV-1 is SEQ ID NO:137.

As noted above, the encoded gp120 amino acid sequence can be a wild-type sequence or a variant sequence. Where the gp120 amino acid sequence is a wild-type sequence, the nucleotide sequence encoding this sequence can be a wild-type nucleotide sequence or one containing one or more “silent” mutations that do not alter the amino acid sequence due to the degeneracy of the genetic code.

For example, if the polynucleotide is intended for use in expressing the encoded polypeptide, silent mutations can be introduced by standard mutagenesis techniques to optimize codons to those preferred by the host cell. Alternatively, a polynucleotide containing silent mutations can be designed and synthesized. More specifically, as those skilled in art understand, codon usages are highly nonrandom and differ between species. Codon usage patterns have been shown to be related to the relative abundance of tRNA isoacceptors. In the case of gp120, changes in codon frequency has been shown to enhance recombinant production of gp120 in mammalians cells. (Haas J et al. (1996) Curr Biol.6(3):315-24; Andre S. et al. (1998) J Virol. 72(2):1497-503.) Native gp120 coding sequences are generally over 60% A-T, while genes that are highly expressed in mammalian systems tend to be about 60% G-C. Accordingly, where the polynucleotides of the invention are intended for use in expressing polypeptides of the invention in mammalian cells, the polynucleotides are preferably “codon-optimized” such that codon frequencies more closely match codon frequencies found in mammalian genes and, more preferably, relatively highly expressed mammalian genes.

Codon-optimized polynucleotides of the invention typically differ in nucleotide sequence from their non-optimized counterparts by about 50% to about 80%. Thus, codon-optimized polynucleotides of the invention may share about 50%, about 60%, about 70%, about 80%, or about 90% sequence identity with the corresponding non-optimized polynucleotide.

In preferred embodiments, polynucleotides of the invention are codon-optimized for expression in mammalian cells. Of particular interest are rodent cells (e.g., mouse, rat, and hamster cells) and primate cells (e.g., monkey or human cells). In an exemplary, preferred embodiment, polynucleotides of the invention are codon-optimized for expression in Chinese hamster ovary (CHO) cells. Polynucleotides of the invention can be codon-optimized based on any gene from the species in which expression is desired, but codon-optimization is preferably carried out by changing the codon frequency to approximate that of relatively highly expressed genes. For example, polynucleotides of the invention can have codon frequencies that approximate those of immunoglobulin genes. Thus, codon frequencies of, e.g., the human Ig Kappa and Mu constant region genes can be determined, and polynucleotides can be engineered to have codon frequencies that approximate the combined codon frequencies of the Ig Kappa and Mu constant region genes. Briefly, the sequences of these genes can be downloaded from GenBank or a similar database. The Ig Kappa C-region is designated as locus HUMIGKC3 in GenBank, and the Ig Mu C-region sequence is designated as locus HSIGMHCC. These sequences can then be translated and codon usage determined. The codon usage information can be combined and combined codon frequencies calculated. A codon-optimized polynucleotide of the invention that has codon frequencies that substantially match the combined codon frequencies for the Ig genes is then conveniently produced synthetically.

In some applications, it is advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH2—NH—O—CH2, CH2—N(CH3)—O—CH2 (known as the methylene(methylimino) or MMI backbone) and CH2—O—N(CH3)—CH2, CH2—N(CH3)—N(CH3)—CH2, and O—N(CH3)—CH2—CH backbones (where phosphodiester is O—P—O—CH2). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

Also, polynucleotides of the invention can be modified to include any of the wide variety of available labels for use in hybridization assays. Suitable labels include those discussed above with respect to labeling gp120 polypeptides of the invention. Fluorescent molecules are conveniently used to label polynucleotides for use in diagnostic methods. Fluorescent labels that can be attached to the polynucleotide include, but are not limited to, fluorescein, texas red, rhodamine, coumarin, oxazine, green fluorescent protein.

Those of skill in the art understand that polynucleotides that are complementary or substantially complementary to the coding strand of polynucleotides of the invention can be employed to inhibit expression of the polypeptides of the invention, which may be of interest for research or therapeutic purposes. Accordingly, the nucleic acids of the invention include such “antisense polynucleotides,” and the phrase “polynucleotide encoding a polypeptide of the invention” is intended to include such antisense molecules. Antisense polynucleotides can be DNA or RNA and are useful in research or therapeutic antisense or RNA interference (RNAi) applications, respectively.

Polynucleotides of the invention can be produced synthetically, amplified from a cloned gp120 polynucleotide or HIV-1 isolate, or otherwise obtained from an HIV-1 isolate. If necessary, the nucleotide sequence of the polynucleotide thus obtained can be altered using any of a variety of cloning and mutagenesis techniques to arrive at the desired nucleotide sequence. See, e.g., Sambrook, J., Fritsch, E. F., and Maniatis, T. (1989) in Molecular Cloning: A Laboratory Manual, Second Edition, Cold Spring Harbor, N.Y.

B. Vectors

A polynucleotide of the present invention can be incorporated into a vector for propagation and/or expression in a host cell. Such vectors typically contain a replication sequence (i.e., an origin of replication) capable of effecting replication of the vector in a suitable host cell (e.g., E. coli, Chinese hamster ovary [CHO] cells) as well as sequences encoding a selectable marker, such as an antibiotic resistance gene. Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art.

If the vector is intended for expression of a polypeptide, the vector includes one or more control sequences capable of effecting and/or enhancing the expression of an operably linked polypeptide-coding sequence. Control sequences that are suitable for expression in prokaryotes, for example, can include a promoter sequence, an operator sequence, and a ribosome binding site. Control sequences for expression in eukaryotic cells can include a promoter, an enhancer, and a transcription termination sequence (i.e., a polyadenylation signal). Expression vectors of the invention are useful for expressing polypeptides of the invention in vivo (e.g., in DNA or virus-derived vaccine applications) or in vitro (e.g., cell culture).

An expression vector according to the invention can also include other sequences, such as, for example, nucleic acid sequences encoding a signal sequence or an amplifiable gene. A signal sequence can direct the secretion of a polypeptide fused thereto from a cell expressing the protein. In the expression vector, nucleic acid encoding a signal sequence is linked to a coding sequence so as to preserve the reading frame of the coding sequence. In addition, the inclusion in a vector of a gene complementing an auxotrophic deficiency in the chosen host cell allows for the selection of host cells transformed with the vector.

Viral vectors are of particular interest for use in delivering polynucleotides of the invention to a cell or organism, followed by expression of the encoded protein. Viral vectors have been extensively studied as a means of delivering polypeptides to an organism to ameliorate a pathological condition, i.e., “gene therapy.” For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1:13-26.

Widely used viral vector systems include, but are not limited to adenovirus, adeno associated virus, vaccinia virus, canary pox virus, herpes viruses, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous genes into most mammalian cells.

Adeno-associated virus (AAV)-based vectors used to transduce cells with target nucleic acids, e.g., in the in vitro production of polynucleotides and peptides, and in vivo and ex vivo gene therapy procedures are described, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351; Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), simian immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to vectors derived from herpes simplex virus (HSV), papillomavirus, Epstein Barr virus (EBV), and lentiviruses.

In one embodiment, a vector according to the invention is a bi-functional plasmid that can serve as a DNA vaccine and a recombinant viral vector. Direct injection of the purified vector DNA into a subject, i.e., as a DNA vaccine, elicits an immune response to encoded polypeptide. The vector can also be used to produce live, recombinant viruses for use in virus-derived vaccines. Such a vector includes a nucleotide sequence encoding a polypeptide of the invention operably linked to two different promoters: an animal promoter (for use of the vector in a DNA vaccine) and a viral promoter (for use in a virus-derived vaccine).

A vector of the present invention is produced by linking desired elements by ligation at convenient restriction sites. If such sites do not exist, suitable sites can be introduced by standard mutagenesis (e.g., site-directed or cassette mutagenesis) or synthetic oligonucleotide adaptors or linkers can be used in accordance with conventional practice.

C. Host Cells

The present invention also provides a host cell containing a vector of the invention. A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, plant cells, and phage, as well as higher eukaryotic cells, including mammalian cells (such as Chinese hamster ovary cells), and, in particular, human cells (such as human embryonic kidney cells). Host cells according to the invention include cells in culture and cells present in live organisms, such as transgenic plants or animals or cells into which a DNA vaccine or viral vector has been introduced.

A vector of the present invention is introduced into a host cell by any convenient method, which will vary depending on the vector-host system employed. Generally, a vector is introduced into a host cell by transformation (also known as “transfection”) or infection with a virus bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

The invention includes the introduction of vectors of the invention into cells in vivo, as well as into cells in vitro, i.e., in cell culture. Techniques for introducing vectors into cells present in a living organism are well known and are described in greater detail below with respect to uses of the immunogenic compositions of the invention. In particular embodiments, vectors of the invention are introduced into a subject in DNA or virus-derived vaccines.

### Recombinant Production Methods

Host cells transformed with expression vectors can be used to express the polypeptides encoded by the polynucleotides of the invention. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

### Immunogenic Compositions

A. Types of Immunogenic Compositions

Immunogenic compositions of the invention can include an isolated polypeptide or an isolated polynucleotide of the invention or both. An isolated gp120 polypeptide of the invention is present in the immunogenic composition in an amount sufficient to elicit an anti-gp120 immune response upon administration of a suitable dose to a subject. An isolated gp120 polynucleotide of the invention is present in the immunogenic composition in a sufficient amount that administration of a suitable dose to a subject results in the expression of an encoded gp120 polypeptide, which stimulates an anti-gp120 immune response.

In preferred embodiments, the immunogenic compositions are “multivalent,” providing at least two different antigenic gp120 sequences. Thus, polypeptide-based compositions can contain one or more polypeptides including gp120 sequences derived from at least two different HIV isolates. Polynucleotide-based compositions can contain one or more polynucleotides encoding gp120 sequences derived from at least two different HIV isolates. Alternatively, immunogenic compositions of the invention can contain at least one polypeptide including a gp120 sequence derived from one HIV isolate and at least one polynucleotide encoding a gp120 sequence derived from a different HIV isolate. Variations of this embodiment can provide as many different antigenic gp120 sequences as desired, for example, 3, 4, 5, 6, 7, 8, 9, 10, 20, 30, 40, 50, or 100 or more different antigenic gp120 sequences.

1. Immunogenic Compositions Containing Polypeptides

The invention provides immunogenic compositions containing an isolated polypeptide of the invention. The compositions optionally contain other components, including, for example, a storage solution, such as a suitable buffer, e.g., a physiological buffer. In a preferred embodiment, the other component is a pharmaceutically acceptable carrier, such as are described in Remington's Pharmaceutical Sciences (1980) 16th editions, Osol, ed., 1980.

The immunogenic composition can include one or more polypeptides of the invention in free form (i.e., unconjugated to any other moiety) or covalently bonded to a cell-specific binding moiety, an immunostimulatory oligonucleotide, or an immunogenic carrier protein.

In one embodiment, an immunogenic composition of the invention includes cells expressing a polypeptide of the invention, a cell lysate, or a fraction thereof, containing the polypeptide, such as, e.g., a membrane fraction.

In another embodiment, an immunogenic composition includes viral particles and/or virally infected cells that display one or more polypeptides of the invention. Immunization of subjects with engineered virus particles and/or infected cells is well known in the art and the compositions used for immunization are generally termed “virus-derived vaccines.” Virus-derived vaccines can be advantageous because the viral infection component can promote a vigorous immune response that activates B lymphocytes, helper T lymphocytes, and cytotoxic T lymphocytes. Numerous viral species can be used to produce recombinant viruses useful in virus-derived vaccines. Examples include vaccinia virus (International Patent Publication WO 87/06262, Oct. 22, 1987, by Moss et al.; Cooney et al., Proc. Natl. Acad. Sci. USA 90:1882-6 (1993); Graham et al., J. Infect. Dis. 166:244-52 (1992); McElrath et al., J. Infect. Dis. 169:41-7 (1994)) and canarypox virus (Pialoux et al., AIDS Res. Hum. Retroviruses 11:373-81 (1995), erratum in AIDS Res. Hum. Retroviruses 11:875 (1995); Andersson et al., J. Infect. Dis. 174:977-85 (1996); Fries et al., Vaccine 14:428-34 (1996); Gonczol et al., Vaccine 13:1080-5 (1995)). Virus-derived vaccines have also been prepared using defective adenovirus or adenovirus (Gilardi-Hebenstreit et al., J. Gen. Virol. 71:2425-31 (1990); Prevec et al., J. Infect. Dis. 161:27-30 (1990); Lubeck et al., Proc. Natl. Acad. Sci. USA 86:6763-7 (1989); Xiang et al., Virology 219:220-7 (1996)). Other viruses that can be engineered to produce recombinant viruses useful in vaccines include retroviruses that are packaged in cells with amphotropic host range (see Miller, Human Gene Ther. 1:5-14 (1990); Ausubel et al., Current Protocols in Molecular Biology, § 9), and attenuated or defective DNA virus, such as, but not limited to, herpes simplex virus (HSV) (see, e.g., Kaplitt et al., Molec. Cell. Neurosci. 2:320-330 (1991)), papillomavirus, Epstein Barr virus (EBV), adeno-associated virus (AAV) (see, e.g., Samulski et al., J. Virol. 61:3096-3101 (1987); Samulski et al., J. Virol. 63:3822-3828 (1989)), and the like.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Preferred embodiments include sustained-release compositions. An exemplary sustained-release composition has a semipermeable matrix of a solid hydrophobic polymer to which the polypeptide is attached or in which the polypeptide is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(-)-3-hydroxybutyric acid. Such matrices are in the form of shaped articles, such as films, or microcapsules.

Exemplary sustained release compositions include polypeptides attached, typically via ε-amino groups, to a polyalkylene glycol (e.g., polyethylene glycol [PEG]). Attachment of PEG to proteins is a well-known means of extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped polypeptide. Liposomes are small vesicles composed of various types of lipids, phospholipids, and/or surfactants. These components are typically arranged in a bilayer formation, similar to the lipid arrangement of biological membranes. Liposomes containing polypeptides are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Compositions of the invention can also include a polypeptide adsorbed onto a membrane, such as a silastic membrane, which can be implanted, as described in International Publication No. WO 91/04014.

Immunogenic compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

2. Immunogenic Compositions Containing Polynucleotides

The invention provides immunogenic compositions containing an isolated polynucleotide encoding a polypeptide of the invention. Such compositions optionally include other components, as for example, a storage solution, such as a suitable buffer, e.g., a physiological buffer. In a preferred embodiment, the other component is a pharmaceutically acceptable carrier as described above.

An alternative to traditional immunization with a polypeptide antigen involves the direct in vivo introduction of a polynucleotide encoding the antigen into tissues of a subject for expression of the antigen by the cells of the subject's tissue. Polynucleotide-based compositions used to vaccinate a subject are termed “DNA vaccines” or “nucleic acid-based vaccines.” DNA vaccines are described in International Patent Publication WO 95/20660 and International Patent Publication WO 93/19183. The ability of directly injected DNA that encodes a viral protein to elicit an immune response has been demonstrated in numerous experimental systems (Conry et al., Cancer Res., 54:1164-1168 (1994); Cox et al., Virol, 67:5664-5667 (1993); Davis et al., Hum. Mole. Genet., 2:1847-1851 (1993); Sedegah et al., Proc. Natl. Acad. Sci., 91:9866-9870 (1994); Montgomery et al., DNA Cell Bio., 12:777-783 (1993); Ulmer et al., Science, 259:1745-1749 (1993); Wang et al., Proc. Natl. Acad. Sci., 90:4156-4160 (1993); Xiang et al., Virology, 199:132-140 (1994)). Studies to assess this strategy in neutralization of influenza virus have used both envelope and internal viral proteins to induce the production of antibodies, but in particular have focused on the viral hemagglutinin protein (HA) (Fynan et al., DNA Cell. Biol., 12:785-789 (1993A); Fynan et al., Proc. Natl. Acad. Sci., 90:11478-11482 (1993B); Robinson et al., Vaccine, 11:957, (1993); Webster et al., Vaccine, 12:1495-1498 (1994)). Vaccination through directly introducing DNA that encodes an HIV env protein to elicit a protective immune response produces both cell-mediated and humoral responses that are analogous to those obtained with live viruses (Raz et al., Proc. Natl. Acad. Sci., 91:9519-9523 (1994); Ulmer, 1993, supra; Wang, 1993, supra; Xiang, 1994, supra). In addition, reproducible immune responses to DNA encoding nucleoprotein that last essentially for the lifetime of the animal have been reported in mice (Yankauckas et al., DNA Cell Biol., 12: 771-776 (1993)). DNA vaccines can be designed to stimulate different arms of the immune system. For example, major histocompatability antigen class I (MHC-I) responses are best stimulated by intracellular expression of protein antigens. In order to accomplish this, the sequences encoding the gp120 signal sequence are deleted and replaced with a codon for an initiator methionine residue. gp120 genes lacking the signal sequence are synthesized on free ribosomes in the cytoplasm, do not acquire N-linked carbohydrate, are proteolytically processed intracellularly, and can stimulate MHC-I-restricted immune responses. MHC-I responses are thought to be particularly effective in promoting cytotoxic T cell responses mediated by CD8-bearing T cells. In contrast, when gp120 genes containing a signal sequence are expressed in mammalian cells, the signal sequence directs synthesis on membrane-bound ribosomes and translocation into the “secretory pathway” where proteins destined for export to the cell surface or extracellular compartment acquire a number of post-translational modifications (e.g., glycosylation) and are presented to the immune system in conjunction with major histocompatability antigens class II (MHC-II) proteins. Protein antigens presented in association with MHC-II antigens are particularly effective in promoting antibody responses and CD4-mediated T cell responses, but are not effective in stimulating CD8-dependent immune responses (e.g., cytotoxic T lymphocytes [CTLs]).

As is well known in the art, a large number of factors can influence the efficiency of expression of antigen genes and/or the immunogenicity of DNA vaccines. Examples of such factors include the vector, the promoter used to drive antigen gene expression, and the stability of the inserted gene in the plasmid. Depending on their origin, promoters differ in tissue specificity and efficiency in initiating mRNA synthesis (Xiang et al., Virology, 209:564-579 (1994); Chapman et al., Nucle. Acids. Res., 19:3979-3986 (1991)). Many DNA vaccines in mammalian systems have relied upon viral promoters derived from cytomegalovirus (CMV). These have had good efficiency in both muscle and skin inoculation in a number of mammalian species.

For pharmaceutical use, polynucleotides of the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides. In a preferred embodiment, therapeutic compositions of the invention include polynucleotides combined with lipids, as described above.

Compositions containing polynucleotides can be stored in any standard form, including, e.g., an aqueous solution or a lyophilized cake. Such compositions are typically sterile when administered to cells or subjects. Sterilization of an aqueous solution is readily accomplished by filtration through a sterile filtration membrane. If the composition is stored in lyophilized form, the composition can be filtered before or after lyophilization and reconstitution.

3. Other Components

In addition to the components described above, immunogenic compositions of the invention can include one or more adjuvants. Exemplary adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and BCG (bacille Calmette-Guerin) and Corynebacterium parvum. In recent years, a new class adjuvants, immunostimulatory oligonucleotides, have been described. These tend to be small (about 15-30-mer) chemically synthesized oligonucleotides rich in guanine and cytosine (GC rich). In some cases (e.g., particulate antigens such as hepatitis B surface antigen), these oligonucleoatides can simply mixed with viral antigens in order to have an immunostimulatory effect. In other cases (e.g., gp120), superior activity is achieved by chemically coupling the oligonucleotides to the protein. gp120 molecules described in this application are particularly well-suited for derivatization with immunostimulatory oligonucleotides by virtue of an unpaired cysteine that is available for chemical coupling to oligonucleotides. Selection of an adjuvant depends on the subject to be vaccinated. Preferably, a pharmaceutically acceptable adjuvant is used. A preferred adjuvant for human subjects is alum (alumina gel).

Other antigens, such as, for example, other HIV-1 polypeptides (e.g., gp41) can be included in the immunogenic compositions of the invention.

Immunogenic compositions can include other polypeptides that enhance the anti-gp120 immune response to the polypeptides of the invention or provide other benefits. For example, it may be advantageous, in particular embodiments to include one or more members of the cytokine family, such as interferons or interleukins, in immunogenic compositions of the invention.

In preferred embodiments, compositions containing an isolated polynucleotide of the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with the polynucleotides of the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above for polypeptides.

In another embodiment, polynucleotides are complexed to dendrimers, which can be used to transfect cells. Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

B. Uses of Immunogenic Compositions

The immunogenic compositions of the invention can be employed to generate a gp120-specific immune response in an animal. Immunogenic compositions of the invention can be administered to the animal by any suitable route of administration, as described in greater detail below.

In one embodiment, an immunogenic composition is administered to an animal to generate anti-gp120 antibodies, e.g., antibodies useful in HIV research. Generally, the animal is one typically employed for antibody production. Mammals (e.g., rodents, rabbits, goats, sheep, etc.) are preferred.

Polyclonal antibodies are raised by injecting (e.g. subcutaneous or intramuscular injection) antigenic polypeptides into a suitable animal (e.g., a mouse or a rabbit). The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the polypeptide to which the antibodies were raised is bound. Those of skill in the art will know of various standard techniques for purification and/or concentration of polyclonal, as well as monoclonal, antibodies see, for example, Coligan, et al. (1991) Unit 9, Current Protocols in Immunology, Wiley Interscience).

For many applications, monoclonal anti-gp120 antibodies are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients, pooling the cells, and fusing the cells with SHFP-1. Hybridomas were screened for production of antibodies that bound to cancer cell lines. Confirmation of specificity among mAb's can be accomplished using routine screening techniques (such as the enzyme-linked immunosorbent assay, or “ELISA”) to determine the elementary reaction pattern of the mAb of interest.

Alternatively, immunogenic compositions of the invention can be used as vaccines for administration to human subjects. In particular, the compositions can be administered to individuals who are not infected with HIV-1 to reduce the risk of, or prevent, infection (prophylaxis of HIV-1 infection). Individuals such as health professionals, police officers, and fire fighters could benefit from prophylactic administration of vaccines of the invention. The compositions can also be administered to individuals who are already infected with HIV-1, but are still able to mount an immune response (see e.g. Salk, Nature 327:473-476 (1987); and Salk et al., Science 195:834-847 (1977)). A so-called “therapeutic vaccine” can ameliorate the existing infection (for example, by improving the subject's condition or slowing or preventing disease progression) and/or can provide prophylaxis against infection with additional HIV-1 strains.

1. Immunogenic Compositions Containing Polypeptides

Polypeptide-based immunogenic composition are conveniently administered by injection (e.g., subcutaneous, intradermal, intramuscular, intraperitoneal, intravenous, etc.), although delivery through catheter or other surgical tubing is also contemplated. Alternative routes include oral administration (tablets and the like) and inhalation (e.g., using commercially available nebulizers for liquid formulations or lyophilized or aerosolized formulations). Polypeptide compositions may also be administered via microspheres, liposomes, immune-stimulating complexes (ISCOMs), or other microparticulate delivery systems or sustained release formulations introduced into suitable tissues (such as blood).

The vaccination dose of gp120 polypeptide administered in the immunogenic composition depends on the properties of the particular composition, e.g., the immunogenicity of a particular formulation, administration route, immunization regimen, condition of the subject and the like, and the determination of a suitable dose for a particular set of circumstances is within the level of skill in the art. Generally, doses of 300 tg of gp120 polypeptide per administration are most preferred, although preferred doses can range from about 10 μg-1 mg per administration, and doses outside of this preferred range can be useful, depending on the particular formulation, administration route (e.g., intramuscular versus subcutaneous), and/or immunization regimen. Different dosages can be used in a series of sequential inoculations. Thus, the practitioner may administer a relatively large dose in a primary inoculation and then boost with relatively smaller doses of gp120 polypeptide.

2. Immunogenic Compositions Containing Polynucleotides

Polynucleotide-based immunogenic compositions of the invention can be employed to express an encoded polypeptide in vivo, in a subject, thereby eliciting an immune response against the encoded polypeptide. Benvenisty, N., and Reshef, L. [PNAS 83, 9551-9555, (1986)] showed that CaPO₄-precipitated DNA introduced into mice intraperitoneally (i.p.), intravenously (i.v.) or intramuscularly (i.m.) could be expressed. The i.m. injection of DNA expression vectors in mice resulted in the uptake of DNA by the muscle cells and expression of the protein encoded by the DNA. The plasmids were maintained episomally and did not replicate. Subsequently, persistent expression has been observed after i.m. injection in skeletal muscle of rats, fish, and primates, and cardiac muscle of rats. WO90/11092 (Oct. 4, 1990) describes the use of naked polynucleotides to vaccinate vertebrates.

Various methods are available for introducing polynucleotides into animals, and the selection of a suitable method for introducing a particular polynucleotide into an animal is within the level of skill in the art. For example, the introduction of gold microprojectiles coated with DNA encoding bovine growth hormone (BGH) into the skin of mice has been shown to elicit anti-BGH antibodies in the mice. A jet injector has been used to transfect skin, muscle, fat, and mammary tissues of living animals. Intravenous injection of a DNA:cationic liposome complex in mice was reported by Zhu et al., [Science 261:209-211 (Jul. 9, 1993)] to result in systemic expression of a cloned transgene. Ulmer et al., [Science 259:1745-1749, (1993)] reported on the heterologous protection against influenza virus infection by intramuscular injection of DNA encoding influenza virus proteins. WO 93/17706 describes a method for vaccinating an animal against a virus, wherein carrier particles were coated with a gene construct and the coated particles are accelerated into cells of an animal. High-velocity inoculation of plasmids, using a “gene-gun,” enhanced the immune responses of mice (Fynan, 1993B, supra; Eisenbraun et al., DNA Cell Biol., 12: 791-797 (1993)), presumably because of a greater efficiency of DNA transfection and more effective antigen presentation by dendritic cells. Polynucleotides of the invention can also be introduced into a subject by other methods known in the art, e.g., transfection, electroporation, microinjection, transduction, cell fusion, DEAE dextran, calcium phosphate precipitation, lipofection (lysosome fusion), or a DNA vector transporter (see, e.g., Wu et al., J. Biol. Chem. 267:963-967 (1992); Wu and Wu, J. Biol. Chem. 263:14621-14624 (1988); Hartmut et al., Canadian Patent Application No. 2,012,311, filed Mar. 15, 1990).

The vaccination dose of gp120 polynucleotide administered in the immunogenic composition depends on the properties of the particular composition, e.g., the immunogenicity of a particular formulation, administration route, immunization regimen, condition of the subject and the like, and the determination of a suitable dose for a particular set of circumstances is within the level of skill in the art. Generally, doses of about 1 to about 100 mg of gp120 polypeptide per administration are preferred. Different dosages can be used in a series of sequential inoculations. Thus, the practitioner may administer a relatively large dose in a primary inoculation and then boost with relatively smaller doses of gp120 polynucleotide.

3. Immunization Regimen

The gp120-specific immune response can be generated by one or more inoculations of an subject with an immunogenic composition of the invention. A first inoculation is termed a “primary inoculation” and subsequent immunizations are termed “booster inoculations.” Booster inoculations generally enhance the immune response, and immunization regimens including at least one booster inoculation are preferred. Any type of immunogenic composition described above may be used for a primary or booster immunization. Thus, for example, an immunogenic composition containing polynucleotides (e.g., or a virus-derived vaccine) of the invention can be used for a primary immunization, followed by boosting with an immunogenic composition containing polypeptides of the invention, or vice versa. In addition, a primary immunization and one or more booster immunization can provide the same antigenic gp120 sequences and/or different antigenic gp120 sequences.

In an exemplary embodiment, a suitable immunization regimen includes at least three separate inoculations with one or more immunogenic compositions of the invention, with a second inoculation being administered more than about two, preferably three to eight, and more preferably approximately four, weeks following the first inoculation. Generally, the third inoculation is administered several months after the second inoculation, and preferably more than about five months after the first inoculation, more preferably about six months to about two years after the first inoculation, and even more preferably about eight months to about one year after the first inoculation. Periodic inoculations beyond the third are also desirable to enhance the subject's “immune memory.” See Anderson et al., J. Infectious Diseases 160(6):960-969 (December 1989).

The adequacy of the vaccination parameters chosen, e.g., formulation, dose, regimen and the like, can be determined by taking aliquots of serum from the subject and assaying antibody titers during the course of the immunization program. Alternatively, the T cell populations can by monitored by conventional methods. In addition, the clinical condition of the subject is be monitored for the desired effect, e.g., prevention of HIV-1 infection or progression to AIDS, improvement in disease state (e.g., reduction in viral load), or reduction in transmission frequency to an uninfected partner or partners. If such monitoring indicates that vaccination is sub-optimal, the subject can be boosted with an addition dose of immunogenic composition, and the vaccination parameters can be modified in a fashion expected to potentiate the immune response. Thus, for example, the dose of gp120 polypeptide or polynucleotide and/or adjuvant can be increased, a gp120 polypeptide can be bonded or complexed to an immunogenic carrier protein, or the route of administration can be changed.

### Diagnostic Methods

A. Detection of Antibodies Specific for gp120 Polypeptides with Unusual Disulfide Structure

The invention provides a diagnostic method for determining whether a subject has produced antibodies specific for a gp120 polypeptide of the invention. The method entails contacting a biological sample from a subject with the gp120 polypeptide of interest and determining whether the sample contains an antibody that specifically binds to the gp120 polypeptide. The sample employed can include any tissue that contains antibodies, but is most conveniently blood or blood fraction (e.g., serum or plasma).

Anti-gp120 antibodies can be detected and/or quantified in the sample using any of a number of well-known immunoassays (see, e.g., U.S. Pat. Nos. 4,366,241; 4,376,110; 4,517,288; and 4,837,168). For a general review of immunoassays, see Methods in Cell Biology Volume 37: Antibodies in Cell Biology, Asai, ed. Academic Press, Inc. New York (1993); Basic and Clinical Immunology 7th Edition, Stites & Terr, eds. (1991).

In a standard solid-phase format, a gp120 polypeptide of interest can be affixed to a solid phase to act as a capture agent that immobilizes any antibody in the sample that is specific for the gp120 polypeptide. Bound antibody can then be separated from free antibody in the sample by a simple washing step.

Immunoassays typically employ a labeling agent to specifically bind to, and label, the binding complex formed by the gp120 polypeptide and any antibody in the sample that is specific for the gp120 polypeptide. Any suitable labeling system, direct or indirect, can be employed. For example, a labeled antibody specific for the species of the subject being tested can be used to label any antibody bound to a solid phase. Other polypeptides capable of specifically binding immunoglobulin constant regions, such as polypeptide A or polypeptide G may also be used as the labeling agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542). Suitable labels include those discussed above with respect to labeling gp120 polypeptides of the invention.

The assays of this invention are scored (as positive or negative or quantity of anti-gp120 antibody) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of anti-gp120 antibody concentration.

In preferred embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen or DNA segment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

B. Detection of gp120 Sequences with Unusual Disulfide Structure

The invention also provides a diagnostic method for determining whether a biological sample from a subject contains a polypeptide including, and/or a polynucleotide encoding, a gp120 amino acid sequence characterized by unusual disulfide structure. Unusual disulfide structure may represent a “transmission phenotype” associated with new infections or a major new variant of HIV-1 in circulation in North America. The method entails assaying the sample for a polypeptide comprising, or a polynucleotide encoding, a gp120 sequence that: (a) lacks one or more cysteine residues at one or more of the following positions: 54, 74, 119, 126, 131, 157, 196, 205, 218, 228, 239, 247, 296, 331, 378, 385, 418, and 445; and/or (b) includes one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510; as numbered from the N-terminal methionine of gp120 from the HXB-2 strain of HIV gp120. In preferred embodiments, the gp120 sequence is not a subtype G gp120 sequence having one or more additional cysteines in the V1 domain or a subtype E gp120 sequence having one or more additional cysteines in the V4 domain. The diagnostic method of the invention can be carried out using any assay that is capable of detecting the presence of, and optionally quantifying, a polypeptide including, and/or a polynucleotide encoding, a gp120 sequence with unusual disulfide structure. The sample employed can include any tissue expected to contain a polypeptide including, or polynucleotide encoding, a gp120 amino acid sequence. Conveniently, blood or a blood fraction (e.g., serum or plasma) is sampled for assay.

1. gp120 Polypeptide-Based Assays

Immunoassays are generally most convenient for detection of gp120 polypeptides characterized by unusual disulfide structure. The considerations for conducting immunoassays to detect gp120 polypeptides, e.g., formats, labeling systems, are essentially as described above with respect to detection of anti-gp120 antibodies.

Preferred immunoassays for detecting gp120 polypeptides are either competitive or noncompetitive. Noncompetitive immunoassays are assays in which the amount of gp120 polypeptide bound to a specific antibody is measured directly. In competitive assays, the amount of gp120 polypeptide in the sample is measured indirectly by measuring the amount of an added (exogenous) polypeptide displaced (or competed away) from the specific antibody.

Antibodies useful in these immunoassays include polyclonal and monoclonal antibodies, which can be produced, for example, as described above.

2. gp120 Polynucleotide-Based Assays

gp120 polynucleotides encoding gp120 sequences characterized by unusual disulfide structure are generally detected based on specific hybridization of a suitable nucleic acid molecule to sample polynucleotides. The nucleic acid molecule specifically hybridizes to a target nucleotide sequence that is present in the gp120 polynucleotide to be detected and not present in other polynucleotides in the sample polynucleotides. In preferred embodiments, the nucleic acid molecule is substantially complementary to the target nucleotide sequence.

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed. In preferred embodiments, gp120 polynucleotides can be obtained from a sample containing HIV-1 viral RNA by reverse transcription, followed by amplification.

i. Amplification-Based Assays

In one embodiment, amplification-based assays can be used to detect, and optionally quantify, a gp120 polypeptide of interest. In such amplification-based assays, the gp120 polynucleotides in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

If it is desirable to determine the level of the gp120 polynucleotide, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

ii. Hybridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of the gp120 polynucleotides can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the gp120 polynucleotide of interest, and standard methods (such as, e.g., densitometry where the nucleic acid probe is radioactively labeled) can be used to quantify the level of the gp120 polynucleotide.)

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low-density” arrays can simply be produced by spotting (e.g. by hand using a pipette) different nucleic acids at different locations on a solid support (e.g. a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a preferred embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can be carried out using a MicroElectroMechanical System (MEMS), such as the Protiveris' multicantilever array.

iii. Detection of gp120 Polynucleotides

gp120 polynucleotides are detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000,7300, 7500, 7700, or 7900 (TaqMan®) from Applied Biosystems or the LightCycler® System from Roche.

The following examples are provided by way of illustration and are not intended to limit the invention.

### EXAMPLES

### Example 1

### Expression of gp120 Polypeptides

HIV-1 gp120 sequences are preferably expressed as fusion proteins containing a heterologous signal sequence and an epitope tag. Exemplary, preferred signal sequences include those from the herpes simplex virus-1 (HSV1) gD glycoprotein and the human tissue plasminogen activator (tPA). The first 29 amino acids of the mature HSV1 gD serves as an epitope tag, which is joined to residue 42 of HIV gp120, as numbered from the N-terminal methionine of the HXB2 strain of gp120 (residue 12 of the mature gp120).

pCI.gD.gp120 contains a mammalian transcription unit and a pUC vector backbone (the pCI mammalian expression vector, which is available commercially from Promega Corporation, Madison Wis.). The transcription unit contains a cytomegalovirus (CMV) immediate early promoter and an artificial intron, followed by the coding region for the signal sequence and the first 29 amino acids of the mature HSV1 gD glycoprotein fused to residue 42 of HIV-1 gp120 at a KpnI site. The gp120 sequence ends with a stop codon and a XhoI restriction site, as noted above. This is followed, in the vector, by an SV40 poly-A sequence and transcription terminator.

pCI.tPA.gp120 also contains a mammalian transcription unit and a pUC vector backbone (the pCI mammalian expression vector, which is available commercially from Promega Corporation, Madison Wis.). The transcription unit contains a CMV immediate early promoter and an artificial intron, followed by the coding region for the first 36 amino acids of the tPA pre-pro sequence and the first 29 amino acids of the mature HSV1 gD fused to residue 42 of HIV-1 gp120 at a KpnI site. The gp120 sequence, which ends with a stop codon and a XhoI restriction site, is followed by an SV40 poly-A sequence and a transcription terminator.

Sequences encoding gp120 polypeptides of the invention are amplified by polymerase chain reaction (PCR) to generate DNA fragments extending from amino acid residue 42 to amino acid residue 529 (residue 18 of gp41). The fragments have a KpnI restriction site at the 5′ end and a stop codon, followed by a XhoI restriction site at the 3′ end. These restriction sites facilitate cloning into the pCI.gD.gp120 vector or the pCI.tPA.gp120 vector.

gp120 amplification products are cleaved with KpnI and XhoI restriction enzymes, and the 1.5 kb gp120 fragments are isolated using a commercially available kit, such as the QIAquick PCR purification kit (Qiagen, Valencia, Calif.). PCI.gD.gp120 or pCI.tPA.gp120 is cleaved with KpnI and XhoI, and the 4.3 kb vector fragment is isolated by a commercially available kit like the QIAquick Gel Extraction kit (Qiagen, Valencia, Calif.). The gp120 fragments are ligated into either vector. The ligation products are transformed into Top10E.coli. DNA is prepared and digested with KpnI and XhoI and subjected to gel electrophoresis to confirm that transformants contain the desired construct. Gene expression is then tested by transient transfection into the 293T embryonic human kidney cell line (Graham et al., J. Gen. Virol. 36:59-77 (1977)) using a calcium phosphate technique (Graham et al., Virology 52:456-467 (1973)) and subsequent assay of the conditioned medium by Western Blot using polyclonal rabbit anti-gp120 antiserum.

### Example 2

### Virus Infectivity and Neutralization Assay

Because in vitro culture of HIV-1 inevitably imposes selective conditions, an accurate representation of viruses circulating in HIV-1 infected patients can only be obtained by examining viral sequences molecularly cloned from patient source materials (e.g. plasma, leukocytes, brain) without an intermediate, in vitro culture step. The resulting cloned complementary DNAs (cDNAs) derived from retroviral RNA genomes can then be inserted into stable DNA-based expression systems for further analysis. One such expression system is the PhenoSense™ HIV neutralization assay (described in detail in Richman et al., 2003). In brief, the assay creates pseudotype viruses expressing cloned HIV-1 envelope proteins, and then utilizes these viruses for viral infectivity studies. This assay has been used to determine the phenotype of cloned HIV envelope glycoproteins with respect to chemokine receptor usage (CXCR4 or CCR5) and sensitivity to soluble CD4, and virus neutralizing antibodies. This assay has been shown to correlate with conventional virus neutralization assays where the ability of antibodies to inhibit activated peripheral blood mononuclear cells (PBMCs) is measured.

The PhenoSense™ assay has advantages over conventional methods in that it is faster, more sensitive, and uses defined viruses. This avoids potential artifacts arising from virus selection in vitro. The PhenoSense™ assay uses nucleic acid amplification (RT-PCR) to derive HIV envelope sequences (gp160) from HIV-positive patient plasma samples. Amplified envelope sequences are incorporated into an expression vector (pCXAS) using conventional cloning methods. Expression vectors can be prepared from single isolated molecular clones or from large pools of sequences that accurately represent the myriad viral quasispecies in the patient at the time of sample collection. Recombinant HIV-1 stocks expressing patient virus envelope proteins (pooled or individual gp160) are prepared by co-transfecting HEK293 cells with a defective HIV-1 genomic viral vector lacking the HIV-1 envelope protein and second expression vector containing the HIV envelope protein of interest. The HIV-1 genomic vector is replication-defective and contains a luciferase expression cassette within a deleted region of the HIV envelope gene. Recombinant viruses pseudotyped with patient virus envelope proteins as well as CXCR4 and CCR5 dependent control viruses (NL4-3, JRCSF) and the specificity control, amphotropic murine leukemia virus (A-MLV), are harvested 48 hours post-transfection and incubated for 1 h at 37° C. with serial 4-fold dilutions of the monoclonal antibodies and/or plasma control. U87 cells that express CD4/CCR5 and cells that express CD4/CXCR4 are inoculated with virus-antibody dilutions. Virus infectivity is determined 72 hours post-inoculation by measuring the amount of luciferase activity expressed in infected cells and recorded as Balanced Relative Light Units (RLUs). Neutralizing activity is displayed as the percent inhibition of viral replication (luciferase activity) at each antibody concentration compared to an antibody negative control. The IC50 is defined as the concentration of monoclonal antibody required to inhibit virus infectivity by 50%. A virus was classified as susceptible to neutralization if the IC50 is at least 3-times higher than the IC50 of the same reagent with the specificity control virus, A-MLV.

### Experimental Results

Three of the cysteine mutants, U-099 (with 19 cysteine residues), U-209 (with 19 cysteine residues), and U-210 (with 20 cysteine residues), were subjected to the PhenoSense™ infectivity and neutralization assay and the results are presented in Table 2. Each of the viruses bound to and infected U87 cells expressing CD4 and the CCR5 chemokine receptor, but not the U87 cells expressing CD4 and the CXCR4 co-receptor, indicating that these mutant viruses are exclusively of the R5 phenotype. The viruses were differentially neutralized using monoclonal antibodies (MAbs) targeting gp41 (4E10 and 2F5) and gp120 (2G12). The 19-cysteine mutant U-099 was 2-8 fold more resistant to the gp41 MAbs, but was neutralized just as well as the other mutants using the gp120 MAb 2G12. Interestingly, U-210 was not neutralized by 2G12, a MAb thought to be broadly cross-neutralizing against primary HIV isolates (see Trkola et al., 1996). However, because 2G12 binding is sensitive to mutations in V4 and U-210 has two additional cysteine residues in this region, neutralization escape may be mediated by these cysteine mutations. The results of this study demonstrate that HIV isolates possessing 19 and 20 cysteine residues, rather than the typical 18 cysteine residues, are functional and can mediate the infection of cells containing CD4 and the CCR5 chemokine receptor.

TABLE 2
IC50 values are defined as the concentration of monoclonal antibody (MAb)
required to inhibit virus infectivity by 50%. The MAbs 4E10 and 2F5 target regions in gp41
while MAb 2G12 recognizes epitopes in gp120. 92HT594 is a dual-tropic virus control.
JRCSF and NL43 are controls for R5 and X4 tropism, respectively. A-MLV is a non-HIV
control that infects either cell type (R5 or X4) but is not inhibited by HIV MAbs. NA; not
applicable.

| Tested on U87 CD4/CCR5 Cells: | | | IC50 | | |
|---|---|---|---|---|---|
| Virus | Growth on R5 Cells? | Tropism of Virus | 4E10 (μg/ml) | 2F5 (μg/ml) | 2G12 (μg/ml) |
| 92HT594 | Yes | Dual | 2.941 | 0.857 | 1.940 |
| U-210 | Yes | R5 | 0.915 | 0.438 | >50.0000 |
| U-209 | Yes | R5 | 0.686 | 1.022 | 2.643 |
| U-099 | Yes | R5 | 5.494 | 2.295 | 1.298 |
| JRCSF | Yes | R5 | 7.911 | 2.104 | 0.764 |
| NL43 | No | X4 | NA | NA | NA |
| A-MLV | Yes | non-HIV | >50 | >50 | >50 |
| Tested on U87 CD4/CXCR4 Cells: | | | | | |
| Virus and Control | | | IC50 | | |
| Tropisms | Growth on X4 Cells? | Tropism of Virus | 4E10 (μg/ml) | 2F5 (μg/ml) | 2G12 (μg/ml) |
| 92HT594 | Yes | Dual | 2.667 | 0.748 | 2.392 |
| U-210 | No | R5 | NA | NA | NA |
| U-209 | No | R5 | NA | NA | NA |
| U-099 | No | R5 | NA | NA | NA |
| JRCSF | No | R5 | NA | NA | NA |
| NL43 | Yes | X4 | 1.415 | 0.477 | 0.473 |
| A-MLV | Yes | non-HIV | >50 | >50 | >50 |

### References

1. Richman D D, Wrin T, Little S J, Petropoulos CJ. (2003). Rapid evolution of the neutralizing antibody response to HIV type 1 infection. Proceedings of the National Academy of Sciences U S A. 100(7): 4144-4149.
2. Trkola A, Purtscher M, Muster T, Ballaun C, Buchacher A, Sullivan N, Srinivasan K, Sodroski J, Moore J P, and Katinger H. (1996). Human monoclonal antibody 2G12 defines a distinctive neutralization epitope on the gp120 glycoprotein of Human Immunodeficiency Virus Type 1. Journal of Virology 70(2): 1100-1108.
All publications and patents mentioned in this specification are herein incorporated by reference to the same extent as if each individual publication or patent were specifically and individually indicated to be incorporated by reference.

1. An immunogenic composition comprising:
(a) an isolated polypeptide comprising, or an isolated polynucleotide encoding, a first gp120 amino acid sequence, wherein the first gp120 sequence comprises at least the V2, V3, and C4 domains of gp120 and:
(i) the first gp120 sequence lacks one or more cysteine residues at one or more of the following positions: 54, 74, 119, 126, 131, 157, 196, 205, 218, 228, 239, 247, 296, 331, 378, 385, 418, and 445; and/or
(ii) the first gp120 sequence comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510;
as numbered from the N-terminal methionine of gp120 from the HXB-2 strain of HIV gp120;
wherein the first gp120 sequence is not a subtype G gp120 sequence having one or more additional cysteines in the V1 domain or a subtype E gp120 sequence having one or more additional cysteines in the V4 domain; and
a pharmaceutically acceptable carrier.

2. The immunogenic composition of claim 1, wherein the first gp120 sequence additionally comprises the V1 domain.

3. The immunogenic composition of claim 1, wherein the immunogenic composition additionally comprises an adjuvant.

4. The immunogenic composition of claim 1, wherein the first gp120 sequence comprises a naturally occurring gp120 sequence.

5. The immunogenic composition of claim 4, wherein the first gp120 sequence comprises a gp120 sequence from a primary isolate of HIV.

6. The immunogenic composition of claim 1, comprising the polypeptide comprising the first gp120 sequence.

7. The immunogenic composition of claim 1, comprising the polynucleotide encoding the first gp120 sequence.

8. The immunogenic composition of claim 1, wherein the first gp120 sequence lacks one or more cysteine residues at one or more of the following positions: 54, 74, 119, 126, 131, 157, 196, 205, 218, 228, 239, 247, 296, 331, 378, 385, 418, and 445.

9. The immunogenic composition of claim 8, wherein the first gp120 sequence lacks one or more cysteine residues at one or more of the following positions: 54, 74, 119, 126, 157, 205, 218, 228, 239, 247, 331, 378, or 385.

10. The immunogenic composition of claim 1, wherein the first gp120 sequence comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510.

11. The immunogenic composition of claim 10, wherein the first gp120 sequence comprises one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120.

12. The immunogenic composition of claim 1, wherein the first gp120 sequence comprises an odd number of cysteines.

13. The immunogenic composition of claim 12, wherein the composition comprises the polypeptide comprising the first gp120 sequence, and a cysteine in the first gp120 sequence is covalently bonded with another polypeptide.

14. The immunogenic composition of claim 13, wherein the covalent bond comprises a disulfide bond.

15. The immunogenic composition of claim 13, wherein the other polypeptide comprises a second gp120 sequence.

16. The immunogenic composition of claim 15, wherein the second gp120 sequence is the same as the first gp120 sequence, said gp120 sequences forming a homodimer.

17. The immunogenic composition of claim 15, wherein the second gp120 sequence is different from the first gp120 sequence, said gp120 sequences forming a heterodimer.

18. The immunogenic composition of claim 13, wherein the other polypeptide comprises a gp41 amino acid sequence.

19. The immunogenic composition of claim 12, wherein the composition comprises the polypeptide comprising the first gp120 sequence, and a cysteine in the gp120 sequence is covalently bonded with an agent selected from the group consisting of a cell-specific binding moiety, a drug, an immunostimulatory oligonucleotide, and an immunogenic carrier protein.

20. The immunogenic composition of claim 1, wherein the polypeptide comprises, or the polynucleotide encodes, a fusion polypeptide comprising the first gp120 sequence.

21. The immunogenic composition of claim 1, wherein the first gp120 sequence has at least about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

22. The immunogenic composition of claim 21, wherein the first gp120 sequence comprises at least the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

23. An isolated polypeptide comprising a first gp120 amino acid sequence, wherein the first gp120 sequence comprises at least the V2, V3, and C4 domains of gp120 and:
(a) the first gp120 sequence lacks one or more cysteine residues at one or more of the following positions: 54, 74, 119, 126, 157, 205, 218, 228, 239, 247, 331, 378, or 385; and/or
(b) the first gp120 sequence comprises one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120,
as numbered from the N-terminal methionine of gp120 from the HXB-2 strain of FHV gp120;
wherein the first gp120 sequence is not a subtype E gp120 sequence having one or more additional cysteines in the V4 domain.

24. The polypeptide of claim 23, wherein the first gp120 sequence additionally comprises the V1 domain.

25. The polypeptide of claim 23, wherein the first gp120 sequence lacks one or more cysteine residues at one or more of the following positions: 54, 74, 119, 126, 157, 205, 218, 228, 239, 247, 331, 378, or 385.

26. The polypeptide of claim 23, wherein the first gp120 sequence comprises one or more additional cysteine residues at a position other than the following positions: 54, 74, 119, 126, 131, 157, 196, 205, 218, 228, 239, 247, 296, 331, 378, 385, 418, or 445, provided that the one or more additional cysteine residues are not present in the V1 domain of gp120.

27. The polypeptide of claim 23, wherein the first gp120 sequence comprises an odd number of cysteines.

28. The polypeptide of claim 27, wherein a cysteine in the gp120 sequence is covalently bonded with another polypeptide.

29. The polypeptide of claim 28, wherein the covalent bond is a disulfide bond.

30. The polypeptide of claim 28, wherein the other polypeptide comprises a second gp120 sequence.

31. The polypeptide of claim 30, wherein the second gp120 sequence is the same as the first gp120 sequence, said gp120 sequences forming a homodimer.

32. The polypeptide of claim 30, wherein the second gp120 sequence is different from the first gp120 sequence, said gp120 sequences forming a heterodimer.

33. The polypeptide of claim 28, wherein the other polypeptide comprises a gp41 amino acid sequence.

34. The polypeptide of claim 27, wherein a cysteine in the gp120 sequence is covalently bonded with an agent selected from the group consisting of a cell-specific binding moiety, a drug, an immunostimulatory oligonucleotide, and an immunogenic carrier protein.

35. The polypeptide of claim 23, wherein the polypeptide comprises a fusion polypeptide comprising the first gp120 sequence.

36. The polypeptide of claim 35, wherein the fusion polypeptide comprises a heterologous signal sequence.

37. The polypeptide of claim 36, wherein the heterologous signal sequence is selected from the herpes simplex virus glycoprotein D (gD-1) signal sequence and the human tissue plasminogen activator signal sequence.

38. The polypeptide of claims 35 or 36, wherein the polypeptide comprises an epitope tag.

39. An isolated polypeptide comprising a first gp120 amino acid sequence, wherein the first gp120 sequence has at least about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

40. The polypeptide of claim 39, wherein the first gp120 sequence comprises at least the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

41. An isolated polynucleotide encoding the polypeptide of any of claims 23-27 and 35-40.

42. The polynucleotide of claim 41, wherein the polynucleotide is codon-optimized for expression in a host cell of a particular species.

43. The polynucleotide of claim 41, wherein the polynucleotide encodes a gp120 sequence that has at least about 99% identity to each of the V1, V2, V3, and C4 domains of a gp120 selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, and 136.

44. The polynucleotide of claim 43, wherein the polynucleotide comprises a gp120 nucleotide sequence selected from the group consisting SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 or a subsequence thereof, wherein the subsequence encodes at least the V1, V2, V3, and C4 domains of gp120.

45. A vector comprising the polynucleotide of claim 41.

46. The vector of claim 45, wherein the vector comprises an expression vector.

47. The vector of claim 45, wherein the vector comprises a viral vector.

48. A host cell comprising the vector of claim 45.

49. The host cell of claim 48, wherein the host cell is selected from a mammalian cell and a bacterial cell.

50. A method of producing a polypeptide comprising a first gp120 amino acid sequence, said method comprising:
(a) introducing the vector of claim 45 into a cell; and
(b) expressing the polypeptide.

51. The method of claim 50, wherein the cell is in vivo.

52. The method of claim 50, wherein the cell is in culture.

53. The method of claim 52, additionally comprising recovering the polypeptide from the culture.

54. A method of producing a polypeptide comprising a first gp120 amino acid sequence, said method comprising:
(a) culturing the host cell of claim 48, wherein the host cell comprises an expression vector, and the host cell is cultured under conditions suitable for expression of the polypeptide; and
(b) recovering the polypeptide from the culture.

55. A method of immunizing an animal with a polypeptide comprising a first gp120 sequence comprising administering the immunogenic composition of claim 1 to the animal.

56. A diagnostic method comprising:
(a) contacting a biological sample from a subject with an isolated polypeptide comprising a gp120 amino acid sequence, wherein the gp120 sequence comprises at least the V2, V3, and C4 domains of gp120 and:
(i) the gp120 sequence lacks one or more cysteine residues at one or more of the following positions: 54, 74, 119, 126, 131, 157, 196, 205, 218, 228, 239, 247, 296, 331, 378, 385, 418, and 445; and/or
(ii) the gp120 sequence comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510;
as numbered from the N-terminal methionine of gp120 from the HXB-2 strain of HIV gp120;
wherein the gp120 sequence is not a subtype G gp120 sequence having one or more additional cysteines in the V1 domain or a subtype E gp120 sequence having one or more additional cysteines in the V4 domain; and
(b) determining whether the biological sample comprises an antibody that specifically binds to the isolated polypeptide.

57. The diagnostic method of claim 56, wherein the gp120 sequence additionally comprises the V1 domain.

58. A diagnostic method comprising assaying a biological sample from a subject to determine whether the sample comprises a polypeptide comprising, or a polynucleotide encoding, a gp120 amino acid sequence that:
(a) lacks one or more cysteine residues at one or more of the following positions: 54, 74, 119, 126, 131, 157, 196, 205, 218, 228, 239, 247, 296, 331, 378, 385, 418, and 445; and/or
(b) comprises one or more additional cysteine residues at a position other than the following positions: 24, 29, 34, 54, 74, 119, 126, 131, 157, 196, 205, 218, 228, 239, 247, 296, 331, 378, 385, 418, 445, 493, 495, 499-501, 503-508, and 510;
as numbered from the N-terminal methionine of gp120 from the HXB-2 strain of HIV gp120.

59. The diagnostic method of claim 58, wherein the gp120 sequence is not a subtype G gp120 sequence having one or more additional cysteines in the V1 domain or a subtype E gp120 sequence having one or more additional cysteines in the V4 domain.

60. The diagnostic method of claim 58, wherein said assaying comprises contacting the sample with an antibody that specifically binds the gp120 sequence under conditions suitable for binding.

61. The diagnostic method of claim 58, wherein said assaying comprises contacting sample polynucleotides with a nucleic acid molecule that hybridizes specifically to a nucleotide sequence encoding the gp120 sequence under conditions suitable for hybridization.

62. The diagnostic method of claim 61, wherein the nucleic acid molecule is one of a pair of amplification primers, said assaying comprises contacting sample polynucleotides with both amplification primers under conditions suitable for amplification, and said determining comprises determining whether an amplification product is produced.

63. The diagnostic method of claim 61, wherein the nucleic acid molecule is a nucleic acid probe affixed to a solid phase.
